(19) United States
(12) Patent Application Publication
(10) Pub. No.: US 2021/0267978 A1
(43) Pub. Date: Sep. 2, 2021

(54) NANOPARTICLE FORMULATIONS OF IKE AND METHODS OF USE THEREOF

(71) Applicant: The Trustees of Columbia University in the City of New York, New York, NY (US)

(72) Inventors: Brent R. Stockwell, New York, NY (US); Yan Zhang, New York, NY (US)

(21) Appl. No.: 17/307,605

(22) Filed: May 4, 2021

Related U.S. Application Data
(63) Continuation of application No. PCT/US2019/060017, filed on Nov. 6, 2019.
(60) Provisional application No. 62/757,006, filed on Nov. 7, 2018.

(51) Int. Cl. A61K 31/517; A61K 9/16; A61P 35/00; A61K 33/243; A61K 31/495; A61K 31/704; A61K 31/675; A61K 31/519; A61K 31/513; A61K 31/395; A61K 31/337; A61K 38/14; A61K 31/573; A61K 31/475; A61K 31/7048; A61K 31/7068; A61K 31/655; A61K 31/282

(52) U.S. Cl. CPC A61K 31/517; A61K 31/282; A61K 9/1641; A61K 9/1694; A61P 35/00; A61K 33/243; A61K 31/495; A61K 31/704; A61K 31/675; A61K 31/519; A61K 31/513; A61K 31/5545; A61K 31/337; A61K 38/14; A61K 31/573; A61K 31/475; A61K 31/7048; A61K 31/7068; A61K 31/655; A61K 9/1647

(57) ABSTRACT

The present disclosure provides, inter alia, nanoparticle formulations comprising nanoparticles of a polymer loaded with a system x_{c}⁻ inhibitor, such as a nanoparticle formulation comprising nanoparticles of PEG-PLGA loaded with IKE or a pharmaceutically acceptable salt thereof. Methods of preparing such nanoparticle formulations, methods of treating cancers in a subject or selectively killing cancer cells using such nanoparticle formulations, and kits comprising such nanoparticle formulations are also provided.

### CROSS REFERENCE TO RELATED APPLICATIONS

The present application is a continuation of PCT international application no. PCT/US2019/060017, filed on Nov. 6, 2019, which claims benefit of U.S. Provisional Patent Application Ser. No. 62/757,006, filed on Nov. 7, 2018, which applications are incorporated by reference herein in their entireties.

### GOVERNMENT FUNDING

This invention was made with government support under grant no. CA209896, awarded by the National Institutes of Health. The government has certain rights in the invention.

### FIELD OF DISCLOSURE

The present disclosure provides, inter alia, nanoparticle formulations comprising nanoparticles of a polymer loaded with a system x_{c}⁻ inhibitor, and methods of use thereof.

### INCORPORATION BY REFERENCE OF SEQUENCE LISTING

This application contains references to amino acids and/or nucleic acid sequences that have been filed concurrently herewith as sequence listing text file “CU19091-seq.txt”, file size of 7 KB, created on Oct. 14, 2019. The aforementioned sequence listing is hereby incorporated by reference in its entirety pursuant to 37 C.F.R. § 1.52(e)(5).

### BACKGROUND OF THE DISCLOSURE

Ferroptosis is a type of regulated cell death driven by iron-dependent lipid peroxidation that can be inhibited by iron chelators and lipophilic antioxidants (Galluzzi et al. 2018; Stockwell et al. 2017). Ferroptosis has a role in regulating survival of some tumor cell types, such as lymphomas, renal cell carcinomas, and hepatocellular carcinomas (Yu et al. 2017; Gout et al. 2001; Liu et al. 2017). Ferroptosis inducers synergize with several chemotherapies, including cisplatin, temozolomide, and doxorubicin in cell culture studies (Sato et al. 2018; Chen et al. 2015; Yamaguchi et al. 2013; Yu et al. 2015). The ferroptosis-suppressing cysteine (Cys₂)/glutamate (Glu) antiporter system x_{c}⁻ is required in some cell contexts for providing cysteine (Stockwell et al. 2017; Yang et al. 2016; Dixon et al. 2012); inhibition of system x_{c}⁻ can induce ferroptosis through cysteine deprivation, subsequent glutathione (GSH) depletion, and ultimately inactivation of glutathione peroxidase 4 (GPX4) (Yang et al. 2014; Dixon et al. 2014). However, existing system x_{c}⁻ inhibitors, including sulfasalazine, glutamate, sorafenib, and erastin, are not suitable for in vivo evaluation due to the lack of potency, selectivity, and/or metabolic stability. IKE is an erastin analog with nanomolar potency, high metabolic stability and intermediate water solubility, thus potentially representing a suitable candidate for in vivo evaluation of the impact of ferroptosis driven through system x_{c}⁻ inhibition in mouse models of cancer (Larraufie et al. 2015).

Diffuse large B cell lymphoma (DLBCL) is an aggressive malignancy of B-lineage lymphocytes, accounting for 30-40% of non-Hodgkin's lymphoma. DLBCL cell lines are particularly sensitive to ferroptosis induced by system x_{c}⁻ inhibition (Yang et al. 2014; Gout et al. 2001; Skouta et al. 2014), due to their inability to use the transsulfuration pathway to synthesize cysteine from methionine, making these cell lines dependent on uptake of cyst(e)ine from the micro-environment (Gout et al. 2001). DLBCL is clinically heterogeneous, with 60% of patients curable with combination therapy, and the remainder succumbing to the disease (Chapuy et al. 2018; Alizadeh et al. 2000). Thus, therapies with distinct mechanisms of action may be beneficial for refractory patients, preventing disease relapse, and improving therapeutic outcomes.

While small molecule system x_{c}⁻ inhibitors are promising agents for inducing ferroptosis in cancers that are addicted to cysteine import, there are a number of potential concerns in translating such agents for therapeutic benefit. First, many compounds, including some system x_{c}⁻ inhibitors, do not accumulate sufficiently in target tumor tissues, resulting in a minimal pharmacodynamic effect; thus, improving delivery and accumulation of such compounds to tumors would be beneficial. Second, while system x_{c}⁻ inhibitors are expected to be largely tolerable based on the observation that SLC7A11 knockout mice have few phenotypes, it is possible that acute inhibition of system x_{c}⁻ would cause toxicity in normal tissues, or that off-target effects of system x_{c}⁻ inhibitors might cause undesirable toxicities.

### SUMMARY OF THE DISCLOSURE

Emerging evidence has shown the potential antitumor effects of using system x_{c}⁻ inhibitors and ferroptosis inducers as single reagents or in combination with chemotherapy in cell culture and some types of xenografts. It is thus important to identify biomarkers and understand the molecular mechanisms of ferroptosis inducers in both tumor cells and cancer models. In the present disclosure, we identify and establish the pharmacokinetic and pharmacodynamic properties of the ferroptosis inducer IKE in a xenograft model, which will be useful for future study of the therapeutic impact of ferroptosis in cancers and other conditions. The present disclosure also identifies a set of genes that are upregulated by IKE treatment in cell culture and demonstrates that the upregulation of these genes is due to system x_{c}⁻ inhibition, as illuminated following co-treatment with ferroptosis inhibitors. The integration of small-molecule tools, untargeted lipidomics, and RT-qPCR provides an efficient means to explore changes in lipid composition and lipid metabolic mechanisms during regulated cell death. Moreover, nanoparticle carriers provide an effective way to improve drug efficacy and reduce systemic toxicity because of targeted localization in tumors. Despite the various nanocarrier systems available and numerous advantages of nanoparticle therapeutics, there are challenges to applying nanocarriers in vivo. One such challenge is to manufacture uniform nanoparticles with high loading capacity at large scale. We utilized a scalable microfluidic platform, which allows for the precise engineering of PEG-PLGA nanoparticles. Given the rapid optimization process and the commercial availability of PEG-PLGA polymers, the nanoparticle system disclosed herein may be useful to other researchers exploring in vivo delivery of ferroptosis inducers.

In the present disclosure, an IKE-loaded nanoparticle (NP) formulation was developed using PEG-PLGA, a well-tolerated di-block copolymer, to achieve enhanced tumor tissue accumulation and improved in vivo delivery of IKE in a DLBCL xenograft model. The IKE PEG-PLGA NPs were formulated using a high flow microfluidic NanoAssemblr, with mean size of ˜80 nm, polydispersity index of 0.2, and loading efficiency of 24% by weight. This IKE NP formulation achieved enhanced accumulation in xenograft tumor tissue, and inhibited tumor growth with less toxicity in mice compared to free IKE. In summary, it was found that the small molecule ferroptosis inducer IKE is able to reduce tumor growth in a DLBCL xenograft model and that IKE PEG-PLGA NPs are suitable for inducing ferroptosis in mouse models.

In view of the foregoing, one embodiment of the present disclosure is a nanoparticle formulation comprising nanoparticles of a polymer loaded with a system x_{c}⁻ inhibitor.

Another embodiment of the present disclosure is a nanoparticle formulation comprising nanoparticles of PEG-PLGA loaded with IKE or a pharmaceutically acceptable salt thereof.

Another embodiment of the present disclosure is a method of preparing a nanoparticle formulation comprising nanoparticles of PEG-PLGA loaded with IKE or a pharmaceutically acceptable salt thereof. This method comprises the steps of: (a) assembling the nanoparticles by employing a NanoAssemblr platform equipped with a high flow microfluidic chip, using the following settings: i) 1:1 ratio of organic to aqueous phases; ii) 25% acetone/75% dimethyl sulfoxide (DMSO) as the organic phase, 10 mg/mL poly(ethylene glycol)-poly(lactic-co-glycolic acid) (PEG-PLGA) in organic phase, and 15% (by weight) IKE to PEG-PLGA polymer in the organic phase; iii) pure water as the aqueous phase; iv) total flow rate of 8 mL/min; and (b) concentrating the assembled nanoparticles by using filter units with concentration factors up to 20.

Yet another embodiment of the present disclosure is a method for treating or ameliorating the effects of a cancer in a subject. This method comprises administering to the subject a therapeutically effective amount of a nanoparticle formulation disclosed herein.

Still another embodiment of the present disclosure is a method for selectively killing a cancer cell. This method comprises contacting the cancer cell with an effective amount of a nanoparticle formulation disclosed herein.

A further embodiment of the present disclosure is a kit comprising a nanoparticle formulation disclosed herein together with instructions for the use of the nanoparticle formulation.

### BRIEF DESCRIPTION OF THE DRAWINGS

The application file contains at least one drawing executed in color. Copies of this patent application with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1F show that IKE is a potent ferroptosis inducer. Data are plotted as the mean±SD, n=2 (FIG. 1B) and (FIG. 1C) or n=3 (FIG. 1E) and (FIG. 1F) biological replicates. Three independent experiments were performed with similar results for FIG. 1B to FIG. 1F.

FIG. 1A shows that the small molecule IKE with isopropoxy, ketone, and imidazole substitutions is more potent than erastin.

FIG. 1B shows that DLBCL cell line sensitivity was measured by incubating cells with a two-fold series dilution of IKE (starting from 100 μM) for 24 hours followed by a Cell Titor-Glo luminescent cell viability test. LY 8, DOHH 2, SUDHL 5, SUDHL 2, SUDHL 6, SUDHL 10, and SUDHL 16 were classified as sensitive cell lines (red). HBL 1, SUDHL 9, WSU-NHL, SUDHL 8, RIVA, and KARPAS 422 were classified as moderately resistant cell lines (black). SUDHL 7, U937, A3/KAW, LY 7, and U2932 were classified as resistant cell lines (blue).

FIG. 1C shows that GSH level was measured with a fluorometric-green probe in SUDHL6 cells treated with different concentrations of IKE with or without 10 μM β-ME for 24 h.

FIG. 1D shows that lipid ROS detected with C11-BODIPY fluorescence were measured in SUDHL6 cells treated with different concentrations of IKE with or without 10 μM fer-1 co-treatment.

FIG. 1E shows that RT-qPCR performed in SUDHL6 cells treated with 500 nM IKE for different periods detected SLC7A11, CHAC1, and PTGS2 mRNA fold change comparing with DMSO treated samples.

FIG. 1F shows that co-treatment of 10 μM Fer-1 with 500 nM IKE for 6 hours prevented PTGS2 upregulation, but not SLC7A11 and CHAC1 upregulation, while 10 μM β-Me co-treatment prevented the upregulation of all three mRNAs.

FIGS. 2A-2B show the heat maps of significantly changed (one-way ANOVA, p<0.05) lipid species in SUDHL-6 cells treated with DMSO, 500 nM IKE, 1 μM IKE, 500 nM IKE with 10 μM fer-1 cotreatment, 500 nM IKE with 10 μM β-Me cotreatment, 500 nM IKE with 10 μM DFO cotreatment, measured by Liquid Chromatography (LC)-MS. Each row represents z-score normalized intensities of the detected lipid species in negative electrospray ionization mode. Each column represents an independent biological replicate. The lipid abundance is color coded with red indicating high signal intensity and dark blue indicating low signal intensity. Abbreviations: PC, phosphatidylcholine; PE, phosphatidylethanolamine; PS, phosphatidylserine, LPC, lysoPC; PE P—, plasmalogen PE; TAG, triacylglycerol; DAG, diacylglycerol, MAG, monoacylglycerol.

FIG. 2C shows the fold change in expression of ACC1, ELOVL7, ATGL, sPLA2F, LPCAT4, LPEAT1, ALOX12, and ALOX15 with 500 nM IKE treatment, 500 nM IKE with 10 μM fer-1 cotreatment, and 500 nM with 10 μM β-Me cotreatment comparing with DMSO control in SUDHL-6 cells. Data are plotted as the mean±SD, n=3 biologically independent samples.

FIG. 2D is a schematic view of fatty acid biosynthesis, lipid remodeling, and arachidonic acid oxidation. The genes upregulated upon IKE treatment are labeled in red.

FIGS. 3A-3G show IKE induced ferroptosis biomarker changes in a lymphoma xenograft model. Data are plotted as the mean±SD, n=3 individual mice (FIG. 3A) and (FIG. 3B).

FIG. 3A is a pharmacokinetic study performed in SUDHL6 subcutaneously xenografted NCG mice measuring IKE accumulation overtime in plasma and tumor using LC-MS.

FIG. 3B shows the analysis of GSH extracted from tumor tissue by fluorometric-green showed over 50% GSH depletion with IKE treatment starting at 4 hours. ***P<0.001, ****P<0.0001 by one-way ANOVA.

FIG. 3C shows that RT-qPCR performed using RNA extracted from tumor tissue showed PTGS2, SLC7A11, and CHAC1 mRNA increase starting from 3 hours, colored as blue in the heatmap. Data are plotted as the mean, n=3 individual mice.

FIGS. 3D-3G show immunofluorescence of dihydropyridine-MDA-lysine adduct in paraffin-embedded tumor samples from mice 4 h after treatment with one dose of vehicle or IKE. Quantification of fluorescence intensity showed 1.6-fold increase of dihydropyridine-MDA-lysine adducts (FIG. 3D) and 1.5-fold increase of 8-OHdG (FIG. 3E) in IKE-treated mouse tumor tissue relative to the vehicle-treated mouse tumor tissue (sections were cut from three mice in each group; five images from each section were captured on Zeiss LSM 800 633/1.40 oil DIC objective). ***p<0.001, ****p<0.0001 by t test. Heatmaps of dysregulated lipids in SUDHL6 subcutaneously xenografted NCG mice treated with one dosage of IKE for at 0, 1, 2, 3, 4, 6, and 24 h or with vehicle, detected by untargeted UPLC-MS analysis. Data shown in heatmaps are lipid species that were identified as being statistically significant (p<0.05) among the groups in (FIG. 3F) negative electrospray ionization mode and (FIG. 3G) positive electrospray ionization mode. Each column represents an independent biological replicate. Each row represents the Z score normalized intensity of a lipid feature. The relative abundance of each identified lipid is color coded in blue, indicating low signal intensity, or red, indicating high signal intensity. Abbreviations: PC, phosphatidylcholine; PE, phosphatidylethanolamine; PI, phosphatidylinositol; PS, phosphatidylserine, LPC, lysoPC; LPE, lysoPE; FA, free fatty acid; TAG, triacylglyceride; DAG, diacylglyceride, MAG, monoacylglyceride.

FIGS. 4A-4D show that IKE PEG-PLGA NPs <100 nm in diameter were formulated and showed reduced toxicity in SUDHL6 subcutaneous-xenografted mice.

FIG. 4A shows that a NanoAssemblr equipped with a microfluidic mixer was used to formulate PEG-PLGA NP (polymer structure shown). The nanoparticle was characterized with a Zetasizer Nano ZS to have a mean diameter of 80 nm, polydispersity index of 0.17, and surface charge of −17 mV.

FIG. 4B shows that IKE PEG-PLGA NP cellular activity was measured by Cell Titor-Glo luminescence cell viability assay in SUDHL 6 cells with 24 hour incubation. The encapsulated IKE in PEG-PLGA NP was measured by LC-MS. The X-axis shows IKE concentration. Data are plotted as the mean±SD, n=2 technical replicates. Three biologically independent experiments were performed with similar results.

FIG. 4C shows that tumor volume fold change compared with day 0, measured by electronic caliper daily and calculated using the formula volume=0.5×length×width², revealed a reduction of tumor growth by IKE 40 mg/kg (n=9), IKE 23 mg/kg (n=11), and IKE NP 23 mg/kg (n=13) treatment for 14 days comparing with vehicle (n=13) and NP vehicle (n=13) controls (data analyzed by two-way ANOVA). Data are plotted as the mean±SD. ns, p>0.5, *p<0.05, **p<0.01, ***p<0.001, ****p<0.0001.

FIG. 4D shows that mouse weight was measured daily and indicated no weight loss upon NP and IKE NP treatments, but weight loss upon IKE 40 mg/kg and IKE 23 mg/kg treatment starting from day 9. Data are plotted as the mean±SD. ns, not significant, *p<0.05, **p<0.01.

FIGS. 5A-5G show that IKE treatment induced lipid peroxidation in tumor tissue during the efficacy study.

FIGS. 5A and 5D show that immunofluorescence (FIG. 8A) and quantification (FIG. 8D) of COX-2 on frozen tumor sections showed 2.0- and 2.8-fold increase in COX-2 intensity upon IKE 23 mg/kg and IKE 40 mg/kg treatment daily for 14 days.

FIGS. 5B and 5E show that immunofluorescence (FIG. 5B) and quantification (FIG. 5E) of MDA on frozen tumor sections measured by confocal microscopy showed 1.4-, 1.8-, 1.8-, and 2.0-fold increase in MDA intensity upon NP vehicle, IKE NP 23 mg/kg, IKE 23 mg/kg, and IKE 40 mg/kg treatment daily for 14 days.

FIGS. 5C and 5F show that immunofluorescence (FIG. 5C) and quantification (FIG. 5F) of 8-OHdG on frozen tumor sections measured by confocal microscopy showed 2.3-, 1.8-, and 2.2-fold increase in 8-OHdG intensity upon IKE NP 23 mg/kg, IKE 23 mg/kg, and IKE 40 mg/kg treatment daily for 14 days. (sections were cut from 5 randomly chosen mice in each group, three images from each section were captured on Zeiss LSM 800 63×/1.40 Oil DIC objective)

FIG. 5G shows that TBARS assay measuring MDA-TBA adducts' fluorescence showed 1.6, 1.4, and 1.8-fold MDA increase upon IKE NP 23 mg/kg, IKE 23 mg/kg and IKE 40 mg/kg treatment. ns P>0.5, *P<0.05, **P<0.01, ***P<0.001, ****P<0.0001 by one-way ANOVA. Data are plotted as the mean±SD.

FIG. 6A shows that IKE-induced DLBCL cell death was rescued by co-treatment with Fer-1. The cell viability was measured by co-treating cells with a two-fold series dilution of IKE (starting from 100 μM) and two-fold series dilution of Fer-1 (starting from 200 μM) for 24 hours followed by a Cell Titor-Glo luminescent cell viability test. The x-axis shows the concentration of IKE and Fer-1 individually. Sensitive (red), moderately resistant (black), and resistant (blue) cell lines were classified by IKE sensitivity test shown in FIG. 1B. Data are plotted as the mean±s.d., n=2 biologically replicates. Three independent experiments were performed with similar results.

FIG. 6B shows the GSH depletion curve measured by incubating SUDHL6 cells with 2-fold series dilution of IKE for 24 hours. The average IC₅₀ of IKE induced GSH depletion by three biological independent experiments was 34 nM.

FIGS. 6C and 6D show that immunofluorescence intensity of MDA in SUDHL6 cells with DMSO or 10 μM IKE treatment for 6 hours indicated 3.4-fold increased MDA upon IKE treatment. Immunofluorescence intensity was quantified on individual cells. ****P<0.0001 by t test.

FIG. 6E shows that co-treatment with DFO inhibited IKE-induced cell death in culture.

FIG. 7 shows the fold change in expression of FASN, ELOVL1, ACSL4, PLA2G6C, LPCAT1, and LPCAT3 with 500 nM IKE treatment, 500 nM IKE with 10 μM fer-1 cotreatment, and 500 nM with 10 μM β-Me cotreatment comparing with DMSO control in SUDHL-6 cells was determined by RT-qPCR.

FIG. 8A is the metscape network analysis of lipids identified in IKE treatment in vivo. Significant accumulated lipids in vivo and significant genes in ferroptosis are highlighted as green circles in the figure.

FIG. 8B is the RT-qPCR analysis of gene expression related to lipid biosynthesis and peroxidation in tumor tissue samples from one dosage of IKE treatment compared with vehicle treatment.

FIG. 8C shows that dose-response curves of SUDHL-6 cells were determined by adding free fatty acids in a 10-point, two-fold dilution series for 24 hours, followed by a Cell Titor-Glo luminescent cell viability test. γ-l-linolenic acid, eicosapentaenoic acid, and arachidonic acid are toxic to cells at high concentration.

FIG. 8D shows that dose-response curves of SUDHL-6 cells were determined by treating with IKE in a 10-point, twofold dilution series in the presence of DMSO or free fatty acids. Relative Area Under Curve (AUC) to DMSO cotreatment was used to indicate the sensitization or protection effect of free fatty acids to IKE-induced ferroptosis.

FIGS. 9A-9G show the optimization of PEG-PLGA NP preparation.

FIG. 9A shows that at a flow rate of 8 mL/min and PEG-PLGA polymer concentration of 10 mg/mL in DMSO, with aqueous:organic (water:DMSO) ratio increases, the average diameter of PEG-PLGA nanoparticles formulated by NanoAssemblr increased.

FIG. 9B shows that at a flow rate of 8 mL/min, PEG-PLGA polymer concentration of 10 mg/mL, and aqueous:organic ratio of 1:1, IKE PEG-PLGA NP prepared from 25% acetone/DMSO has relatively smaller IC₅₀ compared with 0% acetone/DMSO and 50% acetone/DMSO, indicating a higher IKE encapsulation rate in 25% acetone/DMSO. All three solvents produced IKE PEG-PLGA NP with size smaller than 100 nm and polydispersity index (PDI) around 0.2.

FIG. 9C shows that after concentration using filter units with concentration factors up to 20, PEG-PLGA NP's average diameter and PDI weren't significantly increased.

FIG. 9D shows that upon different initial IKE loading in PEG-PLGA NP formulation, 15 wt % IKE showed the highest potency and was used in certain experiments as disclosed further in the Examples.

FIG. 9E shows that with 10 mg/mL PEG-PLGA polymer and 1.5 mg/mL IKE dissolved in 25% acetone/DMSO organic phase and mixed with the water phase at flow rate of 8 mL/min followed by purification with dialysis and concentration with centrifugation, IKE PEG-PLGA NP has average diameter of 82.49 nm and PDI 0.174.

FIG. 9F shows that IKE release rate measured by LC-MS showed controlled release of IKE from PEG-PLGA NP.

FIG. 9G shows that IKE and IKE PEG-PLGA NP-induced DLBCL cell death was rescued by co-treatment with Fer-1. The cell viability was measured by co-treating cells with a two-fold series dilution of IKE (starting from 10 μM) and two-fold series dilution of Fer-1 (starting from 10 μM) for 24 hours followed by a Cell Titor-Glo luminescent cell viability test. The x-axis shows the concentration of both IKE and Fer-1. Data are plotted as the mean±s.d., n=2 biologically replicates. Three independent experiments were performed with similar results.

FIGS. 10A-10B provide the results of IKE efficacy study.

FIG. 10A shows that LC-MS analysis of tumor IKE concentration showed IKE NP 23 mg/kg treated mice have higher tumor IKE accumulation compared with IKE 23 mg/kg group. Tumor samples were from mice treated with saline vehicle, NP vehicle, IKE NP 23 mg/kg, IKE 23 mg/kg, and IKE 40 mg/kg daily for 14 days and sacrificed after 3 hours of the final dosage.

FIG. 10B shows that Western blot analysis of tumor tissue from the efficacy study showed there was no cleaved-caspase 3 increase upon IKE and IKE NP treatment for 14 days. Tumor samples of 9 individual mice from each group were analyzed by western blot.

FIG. 11A shows that caspase 3/7 activity was measured by an Apo-ONE homogenous caspase-3/7 assay in SUDHL-6 cells treated with 500 nM IKE, 500 nM IKE with 10 μM Fer-1 cotreatment, 500 nM IKE with 10 μM β-Me cotreatment, and 10 nM staurosporine for 24 hours.

FIG. 11B shows the analysis of caspase and cleaved-caspase abundance by western blot.

FIGS. 12A-12C show the RT-qPCR performed in SUDHL-6 cells treated with 500 nM IKE for different time.

FIG. 12A shows that IKE induced PTGS2 upregulation very quickly; starting from 5 minutes, there was over 10-fold increased PTGS2 expression.

FIG. 12B shows that SLC7A11 expression was induced by IKE starting from 3 hours post incubation.

FIG. 12C shows that CHAC1 expression was induced by IKE immediately, starting from post 5 minutes, there was over 5-fold increased CHAC1 expression. The highest mRNA change was 33 fold.

FIG. 13 shows the pathways regulating sensitivity of DLBCLs to IKE. DLBCLs have defective transsulfuration pathways resulting in a disability to synthesis cysteine (Cys) thus, DLBCLs heavily rely on the uptake of cysteine from the microenvironment.

FIG. 14 is the schematic overview of IKE-induced ferroptosis.

### DETAILED DESCRIPTION OF THE DISCLOSURE

Ferroptosis is a form of regulated cell death that can be induced by inhibition of the cysteine-glutamate antiporter, system x_{c}⁻, using compounds such as sulfasalazine, sorafenib, erastin, and its more potent and stable analog imidazole ketone erastin (IKE). Few of the existing system x_{c}⁻ inhibitors are suitable for testing the role of ferroptosis in mouse models of cancer, due to lack of selectivity, metabolic stability, and/or potency. IKE is the first potent, metabolically stable, selective inhibitor of system x_{c}⁻ and inducer of ferroptosis potentially suitable for in vivo applications. We hypothesized that IKE-induced ferroptosis would be a viable strategy for targeting some lymphomas based on the high degree of sensitivity of many diffuse large B cell lymphoma (DLBCL) cell lines to erastin (Yang et al. 2014). To improve tumor penetration and the therapeutic index, we simultaneously evaluated biodegradable polyethylene glycol-poly(lactic-co-glycolic acid) nanoparticles (PEG-PLGA NPs) as an IKE formulation. IKE exerted an antitumor effect and inhibited system x_{c}⁻, inhibiting cysteine import, leading to glutathione depletion and lipid peroxidationharmacokinetic-pharmacodynamic (PK-PD) relationships were established for IKE in a xenograft model using the DLBCL cell line SUDHL-6. Intraperitoneal injection of SUDHL-6 into immunocompromised mice with IKE or IKE PEG-PLGA nanoparticles resulted in reduced tumor volume with activation of ferroptosis pharmacodynamic markers. The IKE PEG-PLGA nanoparticle formulation exhibited less toxicity than free IKE, indicating that this formulation is suitable for additional mouse cancer models. Collectively, these results establish that IKE nanoparticles are a potential therapeutic option and research tool for in vivo studies of DLBCL and potentially other ferroptosis-sensitive cancers.

Several studies have shown the important roles of lipids in ferroptosis (Skouta et al., 2014; Yang et al., 2016; Doll et al., 2017; Kagan et al., 2017; Magtanong et al., 2016). The significant depletion of polyunsaturated fatty acids (PUFAs) and PUFA-containing phospholipids has been reported in erastin-treated and erastin-analog-treated HT-1080 cells (Skouta et al., 2014; Yang et al., 2016). Phosphatidylethanolamines (PEs) containing arachidonyl and adrenoyl have been suggested as preferred substrates in RSL3-induced ferroptosis (Kagan et al., 2017; Doll et al., 2017). Moreover, some lipid metabolism genes, including acyl-coenzyme A (CoA) synthetase long-chain family member 4 (ACSL4) and lysophosphatidylcholine acyltransferase 3 (LPCAT3), have been reported as regulators of ferroptosis (Doll et al., 2017; Kagan et al., 2017). We applied untargeted mass spectrometry (MS) in conjunction with ultra-performance liquid chromatography (UPLC) to profile lipid changes in DLBCL cells and a DLBCL xenograft mouse model in response to IKE treatment. We observed significant changes in 62 lipid species in vitro and 37 lipid species in vivo upon IKE treatment, including phospholipids, triacylglycerides (TAGs), diacylglycerides (DAGs), monoacylglycerides (MAGs), and free fatty acids. In addition, a series of genes encoding lipid metabolism enzymes, including the lipid de novo biosynthetic enzymes acetyl-CoA carboxylase 1 (ACC1) and elongation of very long chain fatty acids protein 7 (ELOVL7); lipid-remodeling enzymes adipose triglyceride lipase (ATGL), secretory phospholipase A2f (sPLA2F), lysophosphatidylethanolamine acyltransferase 1 (LPEAT1), and LPCAT4; and lipid peroxidation enzymes lipoxygenases 12 and 15 (ALOX12 and ALOX15), were upregulated upon IKE treatment. Co-treatment with ferroptosis inhibitors ferrostatin-1 (fer-1) or β-mercaptoethanol (β-Me) in IKE-treated cells prevented the upregulation of these genes, indicating that upregulation comes from system x_{c}⁻ inhibition and downstream lipid peroxidation. Collectively, these results suggest the involvement of lipids and lipid metabolism genes in IKE-induced ferroptosis and the protective effect of ferroptosis inhibitors in preventing IKE-induced lipid changes.

Accordingly, one embodiment of the present disclosure is a nanoparticle formulation comprising nanoparticles of a polymer loaded with a system x_{c}⁻ inhibitor.

As used herein, “nanoparticle” or “nanoparticles” are particles between 1 and 100 nanometres (nm) in size with a surrounding interfacial layer. In some embodiments, the size/diameter of the nanoparticles may be varied to achieve the desired clinical effect. The interfacial layer is an integral part of nanoscale matter, fundamentally affecting all of its properties. The interfacial layer typically consists of ions, inorganic and organic molecules. Organic molecules coating inorganic nanoparticles are known as stabilizers, capping and surface ligands, or passivating agents.

As used herein, a “system x_{c}⁻ inhibitor” refers to a compound that decreases the activity of the antiporter which mediates the exchange of extracellular cysteine and intracellular glutamate across the cellular plasma membrane. Non-limiting example of a system x_{c}⁻ inhibitor include sulfasalazine, sorafenib, erastin and analogs thereof.

In some embodiments, the polymer is biodegradable. As used herein, “biodegradable” means that an organic matter is capable of being decomposed by microorganisms, such as bacteria or other living organisms. In some embodiments, the polymer is selected from poly(lactic acid) (PLA), poly(lactide-co-glycolide) (PLGA), and poly(ethylene glycol)-poly(lactic-co-glycolic acid) (PEG-PLGA). Preferably, the polymer is PEG-PLGA.

In some embodiments, the system x_{c}⁻ inhibitor is a small molecule. As used herein, the term “small molecule” has its meaning within the fields of molecular biology and pharmacology, which is a low molecular weight (<900 daltons) organic compound that may regulate a biological process, with a size on the order of 1 nm. Most drugs are small molecules.

In some embodiments, the system x_{c}⁻ inhibitor is an erastin analog, which is selected from the following:

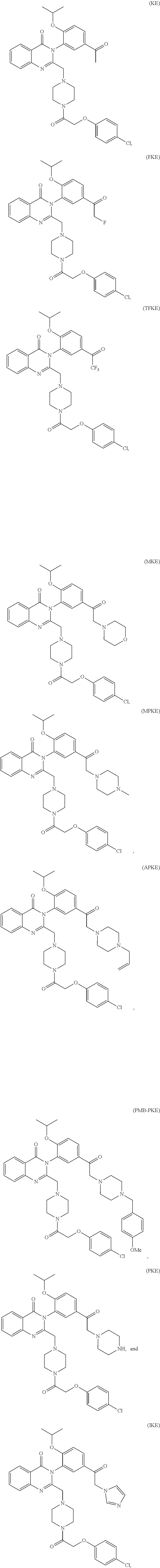

or pharmaceutically acceptable salts thereof. Preferably, the system x_{c}⁻ inhibitor is IKE or pharmaceutically acceptable salts thereof.

In some embodiments, the loaded nanoparticle has a size between 20 nm and 200 nm. In some embodiments, the loaded nanoparticle has a size of about 80 nm. In some embodiments, the loaded nanoparticle has a surface potential of about −17 mV.

In some embodiments, the nanoparticle formulation has a polydispersity index of about 0.2. As used herein, “polydispersity index” or “dispersity” is a measure of the heterogeneity of sizes of molecules or particles in a mixture. Represented by the symbol Ð, it refers to either molecular mass or degree of polymerization. It can be calculated using the equation Ð_{M}=M_{w}/Mₙ, where M_{w} is the weight-average molar mass and Mₙ is the number-average molar mass. It can also be calculated according to degree of polymerization, where Ð_{X}=X_{w}/Xₙ, where X_{w} is the weight-average degree of polymerization and Xₙ is the number-average degree of polymerization.

In some embodiments, the nanoparticle formulation has an encapsulation efficiency of about 24%, although other encapsulation efficiencies may be used so long as the efficacy of the formulation is not adversely effected. As used herein, “encapsulation efficiency” is the percentage of drug that is successfully entrapped into the micelle or nanoparticle. Encapsulation efficiency (EE %) is calculated by (total drug added—free non-entrapped drug) divided by the total drug added.

Another embodiment of the present disclosure is a nanoparticle formulation comprising nanoparticles of PEG-PLGA loaded with IKE or a pharmaceutically acceptable salt thereof. In the present disclosure, the IKE may be loaded onto/into the nanoparticles using any conventional process, including those disclosed herein, including in the Examples below

Another embodiment of the present disclosure is a method of preparing a nanoparticle formulation comprising nanoparticles of PEG-PLGA loaded with IKE or a pharmaceutically acceptable salt thereof. This method comprises, for example, the steps of: (a) assembling the nanoparticles by employing a NanoAssemblr platform equipped with a high flow microfluidic chip, using the following settings: i) 1:1 ratio of organic to aqueous phases; ii) 25% acetone/75% dimethyl sulfoxide (DMSO) as the organic phase, 10 mg/mL poly(ethylene glycol)-poly(lactic-co-glycolic acid) (PEG-PLGA) in organic phase, and 15% (by weight) IKE to PEG-PLGA polymer in the organic phase; iii) pure water as the aqueous phase; iv) total flow rate of 8 mL/min; and (b) concentrating the assembled nanoparticles by using filter units with concentration factors up to 20. As one skilled in the art would understand, the equipment and settings may be varied to achieve nanoparticles having the desired effect.

Yet another embodiment of the present disclosure is a method for treating or ameliorating the effects of a cancer in a subject. This method comprises administering to the subject a therapeutically effective amount of a nanoparticle formulation disclosed herein.

As used herein, the terms “treat,” “treating,” “treatment” and grammatical variations thereof mean subjecting an individual subject to a protocol, regimen, process or remedy, in which it is desired to obtain a physiologic response or outcome in that subject, e.g., a patient. In particular, the methods and compositions of the present disclosure may be used to slow the development of disease symptoms or delay the onset of the disease or condition, or halt or reverse the progression of disease development. However, because every treated subject may not respond to a particular treatment protocol, regimen, process or remedy, treating does not require that the desired physiologic response or outcome be achieved in each and every subject or subject, e.g., patient, population. Accordingly, a given subject or subject, e.g., patient, population may fail to respond or respond inadequately to treatment.

As used herein, the terms “ameliorate”, “ameliorating” and grammatical variations thereof mean to decrease the severity of the symptoms of a disease in a subject.

As used herein, “cancer” means uncontrolled growth of abnormal cells. The present disclosure includes those cancers selected from the following non-limiting group: adrenocortical carcinoma, anal cancer, bladder cancer, bone cancer, brain tumor, breast cancer, carcinoid tumor, carcinoma, cervical cancer, colon cancer, endometrial cancer, esophageal cancer, extrahepatic bile duct cancer, Ewing family of tumors, extracranial germ cell tumor, eye cancer, gallbladder cancer, gastric cancer, germ cell tumor, gestational trophoblastic tumor, head and neck cancer, hypopharyngeal cancer, islet cell carcinoma, kidney cancer, laryngeal cancer, leukemia, lip and oral cavity cancer, liver cancer, lung cancer, lymphoma, malignant mesothelioma, Merkel cell carcinoma, mycosis fungoides, myelodysplastic syndrome, myeloproliferative disorders, nasopharyngeal cancer, neuroblastoma, oral cancer, oropharyngeal cancer, osteosarcoma, ovarian epithelial cancer, ovarian germ cell tumor, pancreatic cancer, paranasal sinus and nasal cavity cancer, parathyroid cancer, penile cancer, pituitary cancer, plasma cell neoplasm, prostate cancer, rhabdomyosarcoma, rectal cancer, renal cell cancer, transitional cell cancer of the renal pelvis and ureter, salivary gland cancer, Sezary syndrome, skin cancer (such as cutaneous t-cell lymphoma, Kaposi's sarcoma, and melanoma), small intestine cancer, soft tissue sarcoma, stomach cancer, testicular cancer, thymoma, thyroid cancer, urethral cancer, uterine cancer, vaginal cancer, vulvar cancer, Wilms' tumor. In some embodiments, the cancer is diffuse large B cell lymphoma (DLBCL).

As used herein, a “subject” is a mammal, preferably, a human. In addition to humans, categories of mammals within the scope of the present disclosure include, for example, agricultural animals, veterinary animals, laboratory animals, etc. Some examples of agricultural animals include cows, pigs, horses, goats, etc. Some examples of veterinary animals include dogs, cats, etc. Some examples of laboratory animals include rats, mice, rabbits, guinea pigs, etc.

In some embodiments, the nanoparticle formulation is administered at up to 750 mg/kg per day.

In some embodiments, the method for treating or ameliorating the effects of a cancer in a subject as disclosed above further comprises co-administering to the subject a chemotherapy drug. Non-limiting examples of a chemotherapy drug include those selected from the group consisting of cisplatin, temozolomide, doxorubicin, cyclophosphamide, methotrexate, 5-fluorouracil, vinorelbine, docetaxel, bleomycin, vinblastine, dacarbazine, mustine, vincristine, procarbazine, prednisolone, etoposide, epirubicin, capecitabine, methotrexate, folinic acid, oxaliplatin, and combinations thereof.

Still another embodiment of the present disclosure is a method for selectively killing a cancer cell. This method comprises contacting the cancer cell with an effective amount of a nanoparticle formulation disclosed herein.

As used herein, “contacting” means bringing, e.g., a compound or composition of the present disclosure into close proximity to, e.g., the cancer cells. This may be accomplished using conventional techniques of drug delivery to mammals or in the in vitro situation by, e.g., providing a compound or composition to a culture media in which the cancer cells are located.

Suitable cells for use in this embodiment may be a mammalian cell, preferably, a human cell. In addition to human cells, categories of mammalian cells within the scope of the present disclosure include, for example, cells from agricultural animals, veterinary animals, laboratory animals, etc. Examples of each type of these animals are as set forth above.

A further embodiment of the present disclosure is a kit comprising a nanoparticle formulation disclosed herein together with instructions for the use of the nanoparticle formulation.

The kits may also include suitable storage containers, e.g., ampules, vials, tubes, etc., for compound or pharmaceutical composition of the present disclosure and other reagents, e.g., buffers, balanced salt solutions, etc., for use in administering the nanoparticle formulation to subjects. The kits may further include a packaging container.

In the present disclosure, an “effective amount” or “therapeutically effective amount” of a compound or composition is an amount of such a compound or composition that is sufficient to effect beneficial or desired results as described herein when administered to a subject or a cell. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of the subject, and like factors well known in the arts of, e.g., medicine and veterinary medicine. In general, a suitable dose of a compound or composition according to the disclosure will be that amount of the compound or composition, which is the lowest dose effective to produce the desired effect with no or minimal side effects.

As used herein, a “pharmaceutically acceptable salt” means a salt of the compounds of the present disclosure which are pharmaceutically acceptable, as defined herein, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

A compound or composition (e.g., a nanoparticle formulation) of the present disclosure may be administered in any desired and effective manner: for oral ingestion, or as an ointment or drop for local administration to the eyes, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Further, a compound or composition of the present disclosure may be administered in conjunction with other treatments. A compound or composition of the present disclosure may be encapsulated or otherwise protected against gastric or other secretions, if desired. The nanoparticle formulation disclosed herein may be presented in unit-dose or multi-dose sealed containers, for example, ampules and vials.

The disclosure is further illustrated by the following examples, which are offered for illustrative purposes, and are not intended to limit the disclosure in any manner. Those of skill in the art will readily recognize a variety of noncritical parameters, which can be changed or modified to yield essentially the same results.

### EXAMPLES

### Example 1

### Methods and Materials

KEY RESOURCES TABLE

| REAGENT or RESOURCE | SOURCE | IDENTIFIER |
|---|---|---|
| Antibodies | | |
| Anti-dihydropyridine-MDA-lysine mouse | Yamada et al. 2001 | N/A |
| mAb 1F83 | | |
| Anti-8-OH-dG antibody | Abcam | Cat# ab62623; |
| | | RRID: |
| | | AB_940049 |
| Anti-xCT antibody | Abcam | Cat# ab37185; |
| | | RRID: |
| | | AB_778944 |
| Anti-cyclooxygenase 2 (COX 2) antibody | Abcam | Cat# ab15191; |
| | | RRID: |
| | | AB _2085144 |
| Goat anti-mouse IgG H&L (Alexa Fluor 647) | Abcam | Cat# |
| | | ab150115; |
| | | RRID: |
| | | AB_2687948 |
| Goat anti-rabbit IgG H&L highly cross- | Thermo Fisher Scientific | Cat# A11034; |
| adsorbed secondary antibody (Alexa | | RRID: |
| Fluor488) | | AB_2576217 |
| Donkey anti-Goat IgG (H + L) cross- | Thermo Fisher Scientific | Cat# A21447; |
| adsorbed, Alexa Fluor 647, polyclonal, | | RRID: |
| secondary antibody | | AB_141844 |
| Anti-15-F2T-isoprostane purified IgG | Oxford Biomedical Research | IS20 |
| Anti-α-tubulin antibody (DM1A) | Santa Cruz Biotechnology | sc-32293 |
| Anti-xCT/SLC7A11 (D2M7A) antibody | Cell Signaling Technology | 12691 |
| Anti-caspase-3 antibody | Cell Signaling Technology | 9662 |
| Chemicals, Peptides, and Recombinant Proteins | | |
| (Poly(ethylene glycol) methyl ether-block- | Sigma-Aldrich | 900948 |
| poly(lactide-co-glycolide) (PEG-PLGA) | | |
| β-mercaptoethanol | Sigma-Aldrich | M3148 |
| Imidazole ketone erastin (IKE) | Larraufie et al. 2015 | N/A |
| Ferrostatin-1 (fer-1) | Skouta et al. 2014 | N/A |
| Critical Commercial Assays | | |
| GSH/GSSG Ratio Detection Assay Kit | Abcam | ab13881 |
| TBARS Assay Kit | Cayman | 700870 |
| BODIPY ™ 581/591 C11 | Thermo Fisher Scientific | D3861 |
| RiboPure ™ RNA Purification Kit | Thermo Fisher Scientific | AM1924 |
| CellTiter-Glo Luminescent Cell Viability | Promega | G7573 |
| Assay | | |
| RNAeasy extraction kit | QIAGEN | 74106 |
| QIAshredder | QIAGEN | 79656 |
| High Capacity cDNA Reverse Transcription | Thermo Fisher Scientific | 4368814 |
| Kit | | |
| Power SYBR Green PCR Master Mix | Thermo Fisher Scientific | 4368702 |
| Other | | |
| ProLong ™ Gold anti-fade Mount with DAPI | Thermo Fisher Scientific | P36962 |
| 10% goat serum | Thermo Fisher Scientific | 50197Z |
| Donkey serum | Sigma-Alderich | D9663 |
| Poly-lysine | Sigma-Aldrich | P4832 |
| Fetal bovine serum | Thermo Fisher Scientific | 10437-028 |
| DMEM | Corning | 10-013 |
| Fetal bovine serum | Life Technologies | 10437036 |
| RPMI-1640 | ATCC | 30-2001 |
| Non-essential amino acids | Thermo Fisher Scientific | 11140076 |
| Penicillin-streptomycin mix | Thermo Fisher Scientific | 15140148 |
| IMDM | Thermo Fisher Scientific | 12440053 |
| HBSS | Thermo Fisher Scientific | 14025076 |
| Steriflip filter unit | Thomas Scientific | 1189Q46 |
| Amicon Ultra-15 centrifugal filter units | Millipore | UF9050 |
| K3 EDTA micro tube | SARSTEDT | 41.1504.105 |
| RIPA buffer | Thomas Scientific | 89900 |
| Software and Algorithms | | |
| Chem Draw Ultra, Version 14.0 | Perkin Elmer | http://www.perkinelmer.com/ |
| | | category/chemdraw |
| Prism, Version 7.0 | Graph Pad Software | https://www.graphpad.com/ |
| | | scientific- |
| | | software/prism/ |
| LipoStar, Version 1.0.4 | Molecular Discovery | https://www.moldiscovery.com/ |
| | | software/lipostar/ |
| MetaboAnalyst, Version 4.0 | MetaboAnalyst | https://www.metaboanalyst.ca/ |
| | | faces/docs/About.xhtml |
| MassLynx, Version 4.1 | Waters | http://www.waters.com/ |
| | | waters/en_US/MassLynx- |
| | | Mass-Spectrometry-Software-/ |
| | | nav.htm?cid= |
| | | 513164&locale= |
| | | en_US |
| XCMS package, Version 3.2.0 | Bioconductor | http://packages. |
| | | renjin.org/package/org.renjin. |
| | | bioconductor/xcms |

### Cell Lines and Media

The SUDHL-5, SUDHL-6, SUDHL-16, and HT-1080 cell lines were obtained from ATCC. The DOHH-2 cell line was obtained from DSMZ. The HBL-1, U2932, SUDHL-7, SUDHL-9, A4/FUK, WSU-DHL, Ly18, Karpas422, SUDHL-1, SUDHL-2, SUDHL-8, SUDHL-10, A3/KAW, RIVA, Ly9, U937, and Ly7 cell lines were provided by Dr. Owen A. O'Connor (Columbia University) and the Columbia Genome Center. HT-1080 cells were grown in DMEM with glutamine and sodium pyruvate (Corning 10-013) supplemented with 10% heat-inactivated fetal bovine serum (FBS) (Life Technologies, 10437036) 1% non-essential amino acids (Thermo Fisher Scientific, 11140076) and 1% penicillin-streptomycin mix (pen-strep) (Thermo Fisher Scientific, 15140148). SUDHL-1, SUDHL-2, SUDHL-6, SUDHL-8, SUDHL-10, SUDHL-16, A3/KAW, RIVA, Ly8 and U937 cells were cultured in RPMI-1640 (ATCC 30-2001) with 10% FBS and 1% pen-strep. DOHH-2, HBL-1, U2932, SUDHL-7, SUDHL-9, A4/FUK, WSU-NHL, and Ly8 were cultured in RPMI-1640 with 10% heat-inactivated FBS and 1% pen-strep. SUDHL-5 and Karpas422 were cultured in RPMI-1640 with 20% FBS and 1% pen-strep. Ly7 was cultured in IMDM (Thermo Fisher Scientific, 12440053) with 15% heat-inactivated FBS and 1% pen-strep. All cells were maintained in a humidified environment at 37° C. and 5% CO₂ in an incubator.

### Animal Studies

The animal studies reported in this disclosure adhere to the ARRIVE guidelines. All animal study protocols were approved by the Columbia University Institutional Animal Care and Use Committee (IACUC). Male NOD-Prkdc^{em26Cd52}//2rg^{em26Cd22}/NjuCrl (NCG) mice (Charles River, strain code 572) mice and NOD.CB17-Prkdcscid/J (NOD SCID) mice (The Jackson Laboratory, stock number 001303) were acclimated after shipping for >3 days before beginning experiments. Mice were fed a standard diet (PicoLab 5053) and maintained with no more than 5 mice per cage.

Pharmacokinetic Analysis in Mice with Three Different Administration Routes

NOD/SCID mice 12-weeks of age and ˜28 g weight were weighed before injection and divided into groups of 3 mice per cage. IKE was dissolved in 5% DMSO/95% Hank's Balanced Salt Solution (HBSS) (Thermo Fisher Scientific, 14025076), pH 4, to create a 5 mg/mL solution. 5% DMSO/95% HBSS at pH 4 solution (Vehicle 1) without IKE was used as vehicle. The solution was sterilized using a 0.22 μm Steriflip filter unit (Thomas Scientific 1189Q46). Mice were dosed using three different routes, IP and PO with 50 mg/kg IKE, and IV with 17 mg/kg IKE. Samples were collected at 0, 1, 3, 4, and 8 h from three mice per time point. Additionally, three mice per group were used as control by administration with equivalent amount of vehicle 1 by IP, PO, and IV, and samples were collected at 8 h. At the appropriate time, mice were sacrificed by CO₂ asphyxiation for 3 min and ˜0.5 mL of blood was collected via cardiac puncture. Blood was immediately put into K3 EDTA micro tube (SARSTEDT 41.1504.105) and placed on ice. Samples were centrifuged for 10 min at 2,100×g at 4° C., plasma was then transferred to a clean tube. Plasma samples were flash frozen in liquid nitrogen and stored at −80° C. IKE was extracted from plasma by adding 900 μL acetonitrile to 100 μL plasma. Samples were mixed for at least 5 min by rotating at room temperature and were sonicated prior to concentration for 10 min at 4,000×g and 4° C. The supernatant was removed and dried on a GeneVac evaporator overnight on an HPLC setting. After drying, the samples were resuspended in 100 μL of methanol and analyzed on the liquid chromatography mass spectrometry (LC-MS), with each sample analyzed twice. Quality control standard samples were prepared by dissolving IKE in 100 μL water and extraction with the same procedures to ensure that the extraction was efficient. LC-MS analysis was performed on a platform comprising a Thermo Scientific Dionex Ultimate 3000RS controlled by Chromeleon (Dionex) and a Bruker amazon SL ESI ion-trap mass spectrometer.

Chromatographic separation was performed at 20° C. on Agient Eclipse Plus C18 column (2.1×50 mm, 3.5 μm) at 20° C. over a 12 minute gradient elution. Mobile phase A consisted of water with 0.1% acetic acid v/v and mobile phase B was methanol with 0.1% acetic acid v/v. After injection, the gradient was held at 80% mobile phase A for 1 min. The gradient was then ramped in a linear fashion to 80% mobile phase over 0.5 min. Over the next 3.5 min, the gradient was ramped in linear fashion to 100% mobile phase B and held at 100% mobile phase B for 3.25 min. The gradient was then ramped in a linear fashion to 80% mobile phase over 0.5 min and held there for the duration of the run. The flow rate was set to 400 μL/min and injection volumes were 5 μL. The retention time for IKE in this gradient was 2.8 min.

Mass spectrometry analysis was performed on a Bruker Amazon SL (Billerca, MA) in positive ESI mode. Trap Control was used to control the ESI settings with the inlet capillary held at −4500 V and the end plate offset at −500 V. Nitrogen was used as the desolvation gas. Hystar v 3.2 was used to integrate the UHPLC and MS applications, and data analysis was performed with the Compass DataAnalysis software. The base peak chromatogram at m/z 655.2 with a width of ±0.1 was integrated and peak area quantified by standard curve.

Pharmacokinetics (PK) of IKE was assessed using Prism fitted with lognormal of one phase exponential decay. The PK parameters are summarized below:

TABLE 1
IKE distribution in plasma through IP,
IV, and PO administration routes

| | Tmax | Cmax | half-life | Tlast | Clast | AUClast |
|---|---|---|---|---|---|---|
| | h | ng/mL | h | h | ng/mL | h*ng/mL |
| IP | 0.31 | 19515 | 1.82 | 8 | 1527 | 53898 |
| IV | 0 | 11384 | 1.31 | 8 | 16 | 16983 |
| PO | 0.72 | 5203 | 0.96 | 8 | 48 | 5723 |

### Pharmacokinetic and Pharmacodynamic Analysis in NCG Mice Bearing SUDHL6 Xenografts

IKE was dissolved in 5% DMSO/95% HBSS at pH 4 to create a 5 mg/mL solution or 3 mg/mL solution. 5% DMSO/95% HBSS at pH 4 was used as vehicle 1. IKE PEG-PLGA nanoparticles and unfunctionalized PEG-PLGA nanoparticles (without IKE) (vehicle 2) prepared with a NanoAssemblr were dialyzed with deionized water overnight, and the water was changed at least twice. Dialyzed IKE-PEG-PLGA nanoparticles and unfunctionalized PEG-PLGA nanoparticles were concentrated by Amicon Ultra-15 Centrifugal Filter Units to create a solution with 80 mg/mL PEG-PLGA nanoparticles. All above solutions were sterilized by filtering through a 0.2 μm syringe filter.

NCG mice 6-weeks of age were injected with 10 million SUDHL-6 cells subcutaneously. Visible tumors appeared after 2 weeks. Tumor size was measured by electronic caliper every 2 days and calculated using the formula: 0.5×length×width². After another 2 weeks, mice were randomly separated into group of 3 mice per cage with roughly tumor size (1200 mm³). Mice were dosed at 50 mg/kg IKE, 22 mg/kg IKE, 22 mg/kg IKE PEG-PLGA nanoparticles (equal to 600 mg/kg PEG-PLGA) using IP at one time and samples were collected at 0, 1, 2, 3, 4, 6, and 24 h with three mice per time point. Mice were dosed with Vehicle 1 and Vehicle 2 (600 mg/kg PEG-PLGA) by IP and samples were collected at 24 h. Mice were euthanized using a CO₂ gas chamber before xenograft dissection and cardio puncture. Plasma samples were collected and analyzed as described above. Tumors were dissected and divided into 6 segments, frozen on dry ice, and stored at −80° C. Before IKE extraction, tumor tissue samples were thawed at room temperature and weighed. A 2.5-fold ratio of volume of phosphate-buffered saline (PBS) (mL/g) was added to the sample and homogenized using Bead Ruptor 4 at speed 4 for 30 sec. The homogenization step was repeated until the samples were homogenized well (final weight/volume was 0.4 g/mL). 100 μL of homogenized tissue (equal to 40 mg) was added to a new microfuge tube, 900 μL of acetonitrile was added. The samples were mixed for at least 5 minutes by rotating on a shaker and were sonicated for at least 30 sec prior to centrifugation for 10 min at 4,000×g and 4° C. The supernatant was removed and dried on the GeneVac overnight on the HPLC setting. After drying, the samples were resuspended in 100 μL of methanol and analyzed on the LC-MS with each sample analyzed twice. Technical replicates were averaged. The concentration of IKE in the sample was determined by comparison against a standard curve of IKE in the range of 25 to 2,500 ng/mL. Pharmacodynamic analysis of ferroptosis biomarkers is described in the RT-qPCR, glutathione, immunofluorescence, and lipidomics sections below. Pharmacokinetics parameters of IKE were assessed using Prism fitted with Lognormal of one phase exponential decay. The PK parameters are summarized below:

TABLE 2
IKE distribution in plasma and tumor
tissue through IP administration

| | Tmax | Cmax | half-life | Tlast | Clast | AUClast |
|---|---|---|---|---|---|---|
| | h | ng/mL | h | h | ng/mL | h*ng/mL |
| Plasma | 1.35 | 5185 | 1.83 | 24 | 30 | 10926 |
| Tumor | 3.30 | 2516 | 3.50 | 24 | 283 | 9857 |

### IKE Efficacy Study

IKE was dissolved in 5% DMSO/95% HBSS at pH 4 to create a 4 mg/mL solution. 5% DMSO/95% HBSS at pH 4 was used as vehicle 1. IKE PEG-PLGA nanoparticles and unfunctionalized PEG-PLGA nanoparticles (without IKE loading) (vehicle 2) prepared with a NanoAssemblr were dialyzed with deionized water overnight, the water was changed at least twice. Dialyzed IKE-PEG-PLGA nanoparticles and unfunctional PEG-PLGA nanoparticles were concentrated by Amicon Ultra-15 Centrifugal Filter Units to create a solution with 80 mg/mL PEG-PLGA nanoparticles. All above solutions were sterilized by filter through a 0.2 μm syringe filter.

NCG mice, 6-weeks old, were injected with 10 million SUDHLO-6 cells subcutaneously. The mice were treated after the tumor size reached 100 mm³. Mice were separated randomly into treatment groups and dosed with 23 mg/kg IKE, 40 mg/kg IKE, 23 mg/kg IKE PEG-PLGA nanoparticles (equal to 700 mg/kg PEG-PLGA), vehicle 1 (based on volume), and vehicle 2 (700 mg/kg PEG-PLGA) once daily by IP for 14 days. Tumor volume was measured daily with electronic caliper and calculated using the formula: 0.5×length×width². 3 h after the final dosage, mice were euthanized with CO₂ and tumor tissue was dissected, weighed, divided into 4-6 segments, frozen, and stored at −80° C. Tumor volume change was analyzed in Prism using one-way ANOVA. Tumor tissues were analyzed as described in the RT-qPCR, immunofluorescence, TBARS, and lipidomics sections below.

### Measurement of DLBCL Lines Sensitivity to IKE

DLBCL cells were plated at 10,000 cells per well in white 384-well plates (32 μL per well) in technical duplicates and incubated overnight. The cells were then treated with 8 μL medium containing a two-fold dilution series of vehicle (DMSO), IKE (starting from 100 μM) with or without Fer-1 (starting from 200 μM). After 24 h incubation, 40 μL of 50% CellTiter-Glo (Promega) 50% cell culture medium was added to each well and incubated at room temperature with shaking for 15 min. Luminescence was measured using a Victor X5 plate reader (PerkinElmer). All cell viability data were normalized to the DMSO vehicle condition. Experiments were performed three independent times with different passages for each cell line. From these data, dose-response curves and IC₅₀ values were computed using Prism 7.0 (GraphPad).

### Measurement of IKE PEG-PLGA Cellular Activity

DLBCL cells or HT-1080 cells were plated at 2,000 cells per well in white 384-well plates (32 μL per well) in technical duplicates and incubated overnight. The cells were then treated with 8 μL medium containing a two-fold dilution series of vehicle 1 (DMSO), IKE, Fer-1, vehicle 2 (unfunctional PEG-PLGA nanoparticles), or IKE PEG-PLGA nanoparticles. The final concentration of IKE in PEG-PLGA nanoparticles were measured by LC-MS as described in the sections below. After 24 h incubation with compounds, 40 μL of 50% CellTiter-Glo (Promega) 50% cell culture medium was added to each well and incubated at room temperature with shaking for 15 min. Luminescence was measured using a Victor X5 plate reader (PerkinElmer). All cell viability data were normalized to the DMSO vehicle condition. Experiments were performed in three independent times with different passages for each cell line. From these data, dose-response curves and IC₅₀ values were computed using Prism 7.0 (GraphPad).

### C-11 BODIPY Lipid Peroxidation Measurement

0.20 million SUDHL-6 cells were seeded in six_{c}⁻ well plates and treated with DMSO, IKE, or Fer-1 at specific concentration. The final cell density of 0.05 million cells/mL. After 24 h, cells were harvested by centrifuging at 300×g for 5 min. Cells were resuspended in 500 μL HBSS containing 2 μM C11-BODIPY (BODIPY 581/591 C11) (Thermo Fisher Scientific, D3861) and incubated at 37° C. for 15 min. Cells were pelleted and resuspended in HBSS. Fluorescence intensity was measured on the FL1 channel with gating to record live cells only (gate constructed from DMSO treatment group). A minimum of 10,000 cells were analyzed per condition.

### Reduced Glutathione Measurement in Cell Culture

2.4 million cells were incubated with DMSO, IKE, or β-mercaptoethanol (β-Me) at a density of 0.2 million cells/mL for 24 h. Cells were collected and washed with cold PBS once. Cell number was counted by Vi-Cell. Cells were resuspended in ice cold RIPA buffer (Thermo Fisher Scientific, 89900) with 100 μL/one million cells. Samples were centrifuged for 15 min at 4° C. at 17,000×g. The resulting supernatant was deproteinized using a trichloroacetic acid and sodium bicarbonate solution and kept on ice. The sample was diluted with assay buffer provided in GSH/GSSG Ratio Detection Assay Kit (Abcam, ab13881) ten-fold. Reduced glutathione (GSH) levels were determined using Fluorometric-Green provided in the kit following the manufacture's protocol. 384-well (Corning) low volume black flat bottom polystyrene non-treated microplates 1230F99 were used in this experiment.

### Reduced Glutathione Measurement in Mouse Tumor Tissue

10 mg of tumor tissue was mixed with 400 μL RIPA buffer, homogenized at speed 5 for 30 sec using a Bead Ruptor 4 (OMNI International). The sample was centrifuged at 17,000×g for 5 min, then the above deprotenization and measurement steps performed.

### TBARS Measurement in Mouse Tumor Tissue

10-25 mg tumor tissue was placed into a 1.5 mL Eppendorf microfuge tube, and the sample was placed on dry ice until use. 20 μL/mg ice cold RIPA buffer with cocktail protease inhibitor (Roche) was added. The tumor tissue was homogenized at speed 5 for 30 sec using Bead Ruptor 4. The sample was centrifuged at 4° C. at 1,600×g for 10 min. The sample was kept on ice and the supernatant used for analysis. Tissue homogenates did not need to be diluted before assaying. TBARS was measured using a TBARS Assay Kit (Cayman 700870) following the manufacture's protocol.

qPCR Analysis of Gene Expression

To perform qPCR analysis in cell culture, DLBCL cells were treated with 0-10 μM IKE, Fer-1, or β-Me for the indicated times. RNA was extracted using the Qiashredder and Qiagen RNeasy Mini kits (Qiagen) according to the manufacture's protocol. 2 μg RNA from each sample was reversed transcribed to cDNA using a High Capacity cDNA Reverse Transcription Kit (Thermo Fisher Scientific, 4368814). Quantitative PCR reactions were performed using Power SYBR Green PCR Master Mix (Applied Biosystems) with triplicate measurement on ViiA 7 Real-Time PCR instrument (Thermo Fischer). HPRT1 was used as an internal reference. Differences in mRNA levels compared with HPRT1 were computed between vehicle and experimental groups using the ΔΔCt method. The primers used in the study were listed below.

| Genes | Foward | Reverse |
|---|---|---|
| HPRT1 | 5′-G000TGGCGTCGTGATTAGTG-3′ | 5′-GCCTCCCATCTCCTTCATCAC-3′ |
| | (SEQ ID NO: 1) | (SEQ ID NO: 2) |
| CHAC1 | 5′- GAA000TGGTTACCTGGGC-3′ | 5′- CGCAGCAAGTATTCAAGGTTGT- |
| | (SEQ ID NO: 3) | 3′ (SEQ ID NO: 4) |
| SLC7A11 | 5′-GGTGGTGTGTTTGCTGTC-3′ | 5′-GCTGGTAGAGGAGTGTGC-3′ |
| | (SEQ ID NO: 5) | (SEQ ID NO: 6) |
| PTGS2 | 5′- TATGTTCTCCTGCCTACTGGAA- | 5′-GCCCTTCACGTTATTGCAGATG- |
| | 3′ (SEQ ID NO: 7) | 3′ (SEQ ID NO: 8) |
| ACSL4 | 5′-0000GCTATCTCTCAGACAC-3′ | 5′-GGTGCTCCAACTCTGCCAG-3′ |
| | (SEQ ID NO: 9) | (SEQ ID NO: 10) |
| FASN | 5′-CTTCCGAGATTCCATCCTACGC- | 5′-TGGCAGTCAGGCTCACAAACG-3′ |
| | 3′ (SEQ ID NO: 11) | (SEQ ID NO: 12) |
| ELOVL1 | 5′-TCCCTCTACATTGTCTATGAGTTC | 5′-TTCAGTTGGCCTTGACCTTGGCAA |
| | C-3′ (SEQ ID NO: 13) | TACC-3′ (SEQ ID NO: 14) |
| ELOVL7 | 5′-AGGATCCATGGCCTTCAGTGATCT | 5′-AACCACCTGCAGCAAATTTGACTC |
| | TACATCGAGG-3′ (SEQ ID NO: | C-3′ (SEQ ID NO: 16) |
| | 15) | |
| ALOX12 | 5′-AGAAAAGTTGACTAGTCCAGTGTG | 5′-AAAAGCTGTGCTAAACCAATTCCG |
| | GTGAA-3′ (SEQ ID NO: 17) | AACAGATTCTCA-3′ (SEQ ID NO: |
| | | 18) |
| ALOX15 | 5′-GGAGCCTTCCTAACCTACAGC-3′ | 5′-CTCACGATTCCTTCCACATACC- |
| | (SEQ ID NO: 19) | 3′ (SEQ ID NO: 20) |
| LPCAT1 | 5′-CACAACCAAGTGGAAATCGAG-3′ | 5′-GCACGTTGCTGGCATACA-3′ |
| | (SEQ ID NO: 21) | (SEQ ID NO: 22) |
| LPCAT3 | 5′-ATCACTGCCGTCCTCACTAC-3′ | 5′-AGTCAACAGCCAAACCAATC-3′ |
| | (SEQ ID NO: 23) | (SEQ ID NO: 24) |
| LPCAT4 | 5′-GGTGGGAGAGAATGCCACTT-3′ | 5′-ATGCAAGGGATGATGGCTGT-3′ |
| | (SEQ ID NO: 25) | (SEQ ID NO: 26) |
| LPEAT1 | 5′-CTGAAATGTGTGTGCTATGAGCG- | 5′-TGGAAGAGAGGAAGTGGTGTCTG- |
| | 3′ (SEQ ID NO: 27) | 3′ (SEQ ID NO: 28) |
| ACC1 | 5′-GAGGGCTAGGTOTTTCTGGAAG- | 5′-CCACAGTGAAATCTCGTTGAGA- |
| | 3′ (SEQ ID NO: 29) | 3′ (SEQ ID NO: 30) |
| sPLA2F | 5′-TGACGACAGGAAAGGAAGCCGCAC | 5′-AGGGAAGAGGGGACTCAGCAACG |
| | -3′ (SEQ ID NO: 31) | AG-3′ (SEQ ID NO: 32) |
| PLA2G6C | 5′-CTGGAACCTGTGTTGGACCT-3′ | 5′-CGGTGATATCTGTGGTCACG-3′ |
| | (SEQ ID NO: 33) | (SEQ ID NO: 34) |
| ATGL | 5′-CAACGCCACTCACATCTACGG-3′ | 5′-GGACACCTCAATAATGTTGGCAC- |
| | (SEQ ID NO: 35) | 3′ (SEQ ID NO: 36) |

qPCR in Mouse Tumor Tissue

To perform qPCR analysis in mouse tumor tissue, ˜20 mg of tumor tissue was prepared and kept on dry ice. The frozen tissue was homogenized with Bead Ruptor 4 at speed 4 for 30 sec. The homogenization was repeated once if there remained unhomogenized tissue. Total mRNA was prepared from the cleared homogenate using Ribopure Kit (Thermo Fisher Scientific, am1924). RNA was reverse transcribed to cDNA then used in quantitative PCR experiment following the same steps used in cell qPCR experiment.

### Immunofluorescence on Cell and Quantification

SUDHL-6 cells were treated with IKE. The cells were harvested by centrifugation and washed with PBS once. The cells were resuspended in PBS, fixed by adding equal volume of 4% paraformaldehyde (PFA), and incubated at room temperature for 15 min. The cells were washed with PBS/0.1% Tween 20 (PBST) twice, resuspended in 10% goat serum (ThermoFisher 50197Z) for 1 h. The cells were incubated with mouse mAb 1F83, which specifically recognizes malondialdehyde (MDA)-lysine adduct 4-methyl-1,4-dihydropyridine-3,5-dicarbaldehyde (MDHDC) (Yamada et al. 2001, Hyvarinen et al. 2014) (1:100 dilution) overnight at 4° C. The cells were washed with PBS three times by centrifugation. The cells were incubated with goat anti-mouse IgG H&L (Alexa Fluor 647) (Abcam ab150115) (1:1000) at room temperature for 1 h. The cells were washed with PBST twice by centrifugation, then resuspended in PBS in 24-well plate with poly-lysine-(Sigma Aldrich P4832)-coated cover slips and centrifuged at 1,000×g for 10 min. ProLong Diamond antifade mountant with DAPI (ThermoFisher P36962) was added to stain the nucleus. All images were captured on a Zeiss LSM 800 confocal microscope at Plan-Apochromat 63×/1.40 Oil DIC objective with constant laser intensity for all samples. When applicable, line-scan analysis was performed on representative confocal microscopy images using Zeiss LSM software to qualitatively visualize fluorescence overlap. The intensity above threshold of the fluorescent signal of the bound antibodies was analyzed using NIH ImageJ software. Data were expressed as fold change compared with the vehicle.

### Immunofluorescence on Frozen Tissue Sections and Quantification

Tumor tissues were fixed in 4% paraformaldehyde (PFA) for 24 h at 4° C. followed by washing with PBS three times. The tissues were perfused in 30% sucrose for 24 h at 4° C. for cryo-protection. The samples were embedded in OCT cryostat sectioning medium, and then moved directly into a cryostat. After equilibration of temperature, frozen tumor tissues were cut into 5 μm thick sections. Tissue sections were mounted on to poly-L-lysine coated slides by placing the cold sections onto warm slides. Slides were stored at −80° C. until staining. For staining, slides were warmed to room temperature followed by washing with PBS twice. A hydrophobic barrier pen was used to draw a circle on each slide. The slides were permeabilized with PBS/0.4% Triton X-100 twice before non-specific-binding blocking by incubating the sections with 10% goat serum (ThermoFisher 50197Z) for 30 minutes at room temperature. The sections were separately incubated with mouse anti-MDA mAb 1F83 (1:1000 dilution), anti-cyclooxygenase 2 (COX 2, AKA PTGS2) antibody (Abcam, ab15191, 1:200 dilution), or anti-8-OH-dG (DNA/RNA damage) antibody (Abcam, ab62623, 1:200 dilution) overnight at 4° C. in humidified chambers. Sections were washed with PBST for twice before incubating with goat anti-mouse IgG H&L (Alexa Fluor 647) (Abcam, ab150115, 1:1000 dilution) or goat anti-rabbit IgG H&L highly cross-absorbed secondary antibody (Alexa Fluor488, Thermo Fisher Scientific, A-11034, 1:1000) at room temperature for 1 h. Slides were then washed twice with PBST. ProLong Diamond antifade mountant with DAPI (ThermoFisher P36962) was added onto slides, which were then covered with the coverslips, sealed by clear fingernail polish and observed under confocal microscopy. All images were captured on a Zeiss LSM 800 confocal microscope at Plan-Apochromat 63×/1.40 Oil DIC objective with constant laser intensity for all analyzed samples. The intensity above threshold of the fluorescent signal of the bound antibodies was analyzed using NIH ImageJ software. Data were expressed as fold change comparing with the vehicle.

### Immunofluorescence on Paraffin-Embedded Tissue Sections and Quantification

Tumor tissue was fixed in 4% paraformaldehyde (PFA) for 24 h at 4° C. followed by washing three times with PBS. The samples were fixed in paraffin. Six series of 5 μM sections were obtained with a sliding microtome. The serial sections were then mounted on gelatin-coated slide. The paraffin-embedded tissue sections were deparaffinized with xylene three times, 5 min each, followed by rehydrating in 100%, 90%, 70%, and 50% ethanol, two washes 5 min each, then rinsed with distilled water. Antigen retrieval was performed in Tris-EDTA buffer, pH 9.0, 95-100° C. for 10 min. Then sections were rinsed in PBST, 2 min each. Then the above steps were followed for staining and quantification.

### Western Blot

10-25 mg tumor tissue was placed into a 1.5 mL Eppendorf microfuge tube, and the sample was placed on dry ice until use. 20 μL/mg ice cold RIPA buffer with cocktail protease inhibitor (Roche) was added. The tumor tissue was homogenized at speed 5 for 30 sec using Bead Ruptor 4. The sample was centrifuged at 4° C. at 1,600×g for 10 min. The sample was kept on ice and the supernatant used for analysis. Caspase-3 antibody which could recognize both full-length caspase-3 and cleaved caspase-3 was used. α-Tubulin antibody was used as the reference.

### IKE PEG-PLGA Nanoparticle Formulation

(Poly(ethylene glycol) methyl ether-block-poly(lactide-co-glycolide) (PEG-PLGA) with PEG average Mₙ 5,000, PLGA Mₙ 15,000, and lactide:glycolide 50:50 was purchased from Sigma-Aldrich (900948). PEG-PLGA nanoparticles were prepared using a microfluidic mixer with the NanoAssemblr Benchtop instrument (Precision NanoSystems Inc., Vancouver, BC) by mixing oil and aqueous phases. PEG-PLGA was dissolved in 25% acetone/75% DMSO at concentration of 10 mg/mL. Milli-Q water was used as aqueous phase. For synthesis of PEG-PLGA nanoparticles, two phases were injected through two inlets of the microfluidic mixer with a speed of 8 mL/min and a volume ratio of 1:1. Nanoparticles were collected in the sample collection tube by discarding an initial and final waste volume of 0.25 and 0.05 mL. Samples were dialyzed in at least 500× deionized water overnight using 10 kD cut-off dialysis bag (ThermoFisher, SnakeSkin Dialysis Tubulin, 68100) and the water was changed twice in between to remove organic solvent. Then the purified nanoparticles were concentrated by centrifuging using Amicon Ultra-15 Centrifugal Filter Units (Millipore, UF9050) at 2,168×g to the desired concentration. For encapsulation of IKE, IKE was dissolved in a solution of PEG-PLGA in 25% acetone and 75% DMSO. The final concentration of PEG-PLGA was 10 mg/mL and IKE is 1.5 mg/mL. The process was the same with PEG-PLGA nanoparticles preparation. Nanoparticle size and potential were measured using a Malvern Zetasizer Nano ZS.

### IKE Quantification in PEG-PLGA Nanoparticles

IKE concentration was determined in PEG-PLGA nanoparticles using Liquid Chromatography-Mass Spectrometry (LC-MS). IKE was extracted from nanoparticles by mixing 100 μL of samples with 900 μL of acetonitrile. After mixing for 1 h, the mixture was sonicated for 5 min and then centrifuged at 4000 rpm for 10 min. The supernatant was transferred to a new vial. The organic solvent was removed by Genevac evaporation. The residues were re-dissolved in 100 μL MeOH in a LC-MS vial. Calibration standards and quality control samples were prepared spanning a range of 25 ng/ml to 1250 ng/ml IKE in MeOH. Peak integration and data analysis were performed. Using the standard curve, IKE concentration in the sample was determined.

Kinetics of IKE Release from PEG-PLGA Nanoparticles

200 μL newly generated IKE PEG-PLGA nanoparticles were mixed with 800 μL PBS solution. They were incubated on a shaker at 37° C. Samples were collected followed by centrifugation at 10,000×g for 10 min. The obtained supernatant was evaporated using a GeneVac. The obtained residues were resuspended in 100 μL methanol and analyzed with LC-MS with the methods described above. Peak integration and data analysis were performed to quantify released IKE concentration in the samples.

### Erastin and IKE Solubility Measurements

Solubility measurement samples were prepared in a 96-well Corning Costar plate with two at 1% and 5% DMSO v/v with a total sample volume of 200 μL in MilliQ water. A BMG Labtech NEPHELOstar nephelometer was used to measure turbidity of the samples with the following parameters per the instrument manufacturer: Gain=80, Laser Focus=2.50 mm, and Laser Intensity=90%. Samples were shaken in the nephelometer by orbital shaking for 3 s prior to the turbidity measurement. Sample values were blanked in the instrument software using the corresponding water and DMSO condition values. Turbidity 4000 NTU Calibration Standard-Formazin (Millipore-Sigma) was used as a positive control for all turbidity measurements. The turbidity of each sample was measured within 20 minutes of initial preparation. After the initial measurement, the sample plates were covered with aluminum sealing film and allowed to sit overnight at room temperature prior to re-measuring.

### IKE Synthesis

IKE was synthesized using methods previously reported (Larraufie et al. 2015) except that the reduction step of nitrobenzene to aniline was modified, which is described here. To a solution of substituted nitrobenzene (1 equiv.) in MeOH (0.1 M), Pd/C (0.2 equiv.) were added. The reaction mixture was gas exchanged with hydrogen, then stirred at room temperature overnight under 1 atm hydrogen gas. Upon completion, the mixture was filtered with celite and concentrated under vacuo. The resulting mixture was purified by combiflash 0-10% methanol/dichloromethane to afford the corresponding aniline. The characterization of the final product IKE was:

1H NMR (400 MHz, Dimethyl sulfoxide-d6) δ 8.31 (ddd, J=8.0, 1.5, 0.6 Hz, 1H), 8.07 (dd, J=8.7, 2.3 Hz, 1H), 8.01 (d, J=2.2 Hz, 1H), 7.83 (ddd, J=8.4, 7.0, 1.5 Hz, 1H), 7.80-7.73 (m, 1H), 7.69 (s, 1H), 7.54 (ddd, J=8.2, 7.0, 1.3 Hz, 1H), 7.29-7.21 (m, 2H), 7.25-7.13 (m, 2H), 6.98 (s, 1H), 6.93-6.83 (m, 2H), 5.48 (d, J=17.6 Hz, 1H), 5.36 (d, J=17.6 Hz, 1H), 5.32 (s, 2H), 4.78-4.68 (m, 1H), 4.63 (s, 2H), 3.62 (s, 1H), 3.44 (d, J=16.3 Hz, 4H), 3.29 (d, J=14.0 Hz, 1H), 3.22 (d, J=14.0 Hz, 1H), 2.50-2.13 (m, 4H), 1.28 (dd, J=16.2, 6.0 Hz, 6H). MS (m/z): [M]⁺ calculated for C₃₅H₃₅ClN₆O₅, 655.14; found 655.24. HPLC detected purity: 99%.

### Mass Spectrometry-Based Untargeted Lipidomics Analysis

### Sample Preparation In Vitro Study

The lipids were extracted from each cell sample using a modified Matyash method (Matyash et al. 2008) as described previously (Cajka et al. 2014). 5 million cells treated with DMSO, 1 μM IKE, 500 nM IKE with or without 10 μM fer-1 and 10 μM β-Me for 24 hours were homogenized in 250 μL cold methanol containing 0.1% butylated hydroxyl toluene (BHT) with micro tip sonicator. Homogenized samples were transferred to fresh glass tubes containing 850 μL of cold methyl-tert-butyl ether (MTBE) and vortex-mixed for 30 sec. To enhance extraction efficiency of lipids, the samples were incubated overnight at 4° C. on the shaker. On the next day, 200 μL of cold water was added to each sample, and incubated for 20 min on ice before centrifugation at 3,000 rpm for 20 min at 4° C. The organic layer was collected followed by drying under a gentle stream of nitrogen gas on ice and stored at −80° C. until LC-MS analysis. The protein pellet was used to measure protein concentration using Bio-Rad protein assay. The samples were re-constituted in a solution containing IPA/ACN/water (4:3:1, v/v/v) containing mixture of internal standard for further MS analysis. A quality control (QC) sample was prepared by combining 40 μL of each sample to assess the reproducibility of the features through the runs.

### Ultra-Performance Liquid Chromatography Analysis

Chromatographic separation of extracted lipids was carried out at 55° C. on Acquity UPLC HSS T3 column (2.1×150 mm, 1.8 μm) over a 17-min gradient elution. Mobile phase A consisted of ACN/water (60:40, v/v) and mobile phase B was IPA/ACN/water (85:10:5, v/v/v) both containing 10 mM ammonium acetate and 0.1% acetic acid. After injection, the gradient was held at 60% mobile phase A for 1.5 min. For the next 12 min, the gradient was ramped in a linear fashion to 100% B and held at this composition for 3 min. The eluent composition returned to the initial condition in 1 min, and the column was re-equilibrated for an additional 1 min before the next injection was conducted. The flow rate was set to 400 μL/min and Injection volumes were 5 μL using the flow-through needle mode in both positive and negative ionization modes. The QC sample was injected between the samples and at the end of the run to monitor the performance and the stability of the MS platform. This QC sample was also injected at least 5 times at the beginning of the UPLC/MS run, in order to condition the column.

### Mass Spectrometry Analysis

The Synapt G2 mass spectrometer (Waters, Manchester, U.K.) was operated in both positive and negative electrospray ionization (ESI) modes. For positive mode, a capillary voltage and sampling cone voltage of 3 kV and 32 V were used. The source and desolvation temperature were kept at 120° C. and 500° C., respectively. Nitrogen was used as desolvation gas with a flow rate of 900 L/hr. For negative mode, a capillary voltage of −2 kV and a cone voltage of 30 V were used. The source temperature was 120° C., and desolvation gas flow was set to 900 L/hr. Dependent on the ionization mode the protonated molecular ion of leucine encephalin ([M+H]⁺, m/z 556.2771) or the deprotonated molecular ion ([M−H]⁻, m/z 554.2615) was used as a lock mass for mass accuracy and reproducibility. Leucine enkephalin was introduced to the lock mass at a concentration of 2 ng/μL (50% ACN containing 0.1% formic acid), and a flow rate of 10 μL/min. The data was collected in duplicates in the centroid data independent (MS^{E}) mode over the mass range m/z 50 to 1600 Da with an acquisition time of 0.1 seconds per scan.

The QC samples were also acquired in enhanced data independent ion mobility (IMS-MS^{E}) in both positive and negative modes for enhancing the structural assignment of lipid species. The ESI source settings were the same as described above. The traveling wave velocity was set to 650 m/s and wave height was 40 V. The helium gas flow in the helium cell region of the ion-mobility spectrometry (IMS) cell was set to 180 mL/min to reduce the internal energy of the ions and minimize fragmentation. Nitrogen as the drift gas was held at a flow rate of 90 mL/min in the IMS cell. The low collision energy was set to 4 eV, and high collision energy was ramping from 25 to 65 eV in the transfer region of the T-Wave device to induce fragmentation of mobility-separated precursor ions.

### Data Pre-Processing and Statistical Analysis

All raw data files were converted to netCDF format using DataBridge tool implemented in MassLynx software (Waters, version 4.1). Then, they were subjected to peak-picking, retention time alignment, and grouping using XCMS package (Smith et al. 2006) (version 3.2.0) in R (version 3.5.1) environment. For the peak picking, the CentWave algorithm (Tautenhahn et al. 2008) was used with the peak width window of 2-25 s. For peak grouping, bandwidth and m/z-width of 2 s and 0.01 Da were used, respectively. After retention time alignment and filling missing peaks, an output data frame was generated containing the list of time-aligned detected features (m/z and retention time) and the relative signal intensity (area of the chromatographic peak) in each sample. Technical variations such as noise were assessed and removed from extracted features' list based on the ratios of average relative signal intensities of the blanks to QC samples (blank/QC>1.5). Also, peaks with variations larger than 30% in QCs were eliminated. Multivariate and univariate analyses were performed using MetaboAnalyst (Chong et al. 2018) (version 4.0) and in R (version 3.5.1) environment. Group differences were calculated using VIP scores of PLS-DA model and one-way ANOVA (p<0.05) and false discovery rate of 5% to control for multiple comparisons.

### Structural Assignment of Identified Lipids

Identification and structural characterization of significant lipid features were confirmed with LipoStar (Goracci et al. 2017) (Version 1.0.4, Molecular Discovery, UK). Lipidomix standard (Avanti Polar Lipids, INC., Alabaster, Ala., USA) and quality control samples were analyzed in LipoStar with the recommended data processing parameters (Goracci et al. 2017) except MS/MS signal filtering threshold was set to 20 for both positive and negative ionization mode. The precursor ion (MS) and fragment ion information obtained by data independent MS (MSE) were automatically annotated using LipoStar database library with mass tolerances of 5 ppm and 10 ppm, respectively. Annotated lipid species with the highest score and high-confidence identification (matches between experimental and theoretical MS/MS spectra) were approved, and the identified lipids with low-confidence matches were further evaluated manually using MS^{E} data viewer (Version 1.3, Waters Corp., MA, USA). In case of co-eluting and isomeric lipid species (e.g., triacylglycerols), all the available fragments in MS/MS spectra data were reported. However, if fragments for fatty acyl compositions of lipids weren't clear, the lipid species were annotated as sum of total number of carbons and double bonds (e.g. PC 32:2).

### Untargeted Lipidomics Study In Vivo

25 mg frozen tissue was homogenized in 15 μL/mg pre-chilled methanol containing 0.1% butylated hydroxyl-toluene (BHT) at speed 5 for 30 secs using a Bead Ruptor 4 (OMNI International). Then, 300 μL of tissue lysate were transferred to a glass vials containing 1,000 μL ice-cold methyl-tert-butyl ether (MTBE) and vortex-mixed for 30 sec. The sample was stored in the −20° C. freezer overnight to enhance lipids extraction. In the following day, 250 μL of ice-cold methanol was added to each sample and vortex-mixed for 30 sec vigorously. Then, samples were incubated on dry ice for 20 min on the shaker followed by centrifuge at 3,000 rpm for 20 min at 4° C. Finally, 1,000 μL of upper phase containing lipids were transferred to fresh glass vials, and evaporated to dryness under the stream of N₂ gas. The samples were re-constituted in a solution containing IPA/ACN/water (4:3:1, v/v/v) containing mixture of internal standard for further MS analysis. The other steps including LC-MS analysis and data processing are the same as in vitro lipidomic analysis as mentioned above. MetScape analysis KEGG ID and HMDB ID are as below:

| | KEGG or | |
|---|---|---|
| Lipids | HMDB ID | Full name |
| FA 14:0 | C06424 | Myristic acid |
| FA 16:1 | C08362 | Palmitoleic acid |
| FA 18:1 | C00712 | Oleic acid |
| FA 18:2 | C01595 | Linoleic acid |
| FA 18:3 | C06426 | Gamma-Linolenic acid |
| FA 20:1 | C16526 | Eicosenoic acid |
| FA 20:2 | C16525 | Eicosadienoic acid |
| FA 20:3 | C03242 | 8,11,14-Eicosatrienoic acid |
| FA 20:4 | C00219 | Arachidonic acid |
| FA 20:5, EPA | C06428 | Eicosapentaenoic acid |
| FA 22:1 | C08316 | Erucic acid |
| FA 22:4 | C16527 | Adrenic acid |
| FA 22:5 | C16513 | Docosapentaenoic acid |
| FA 22:6 | C06429 | Docosahexaenoic acid |
| FA 22:0 | C08281 | Behenic acid |
| FA 24:0 | C08320 | Tetracosanoic acid |
| LPC 18:1 | HMDB0002815 | LysoPC(18:1(11Z)) |
| LPE 18:1 | HMDB0011506 | LysoPE(18:1(9Z)/0:0) |
| PC 16:1/16:1 | HMDB0008002 | PC(16:1(9Z)/16:1(9Z)) |
| PI 18:2/20:3 | HMDB0009854 | PI(18:2(9Z,12Z)/20:3 |
| | | (5Z,8Z,11Z)) |
| FA 16:0 | C00249 | Palmitic acid |
| FA 17:0 | HMDB0002259 | Heptadecanoic acid |
| FA 18:0 | C01530 | Stearic acid |
| FA 24:1 | C08323 | Nervonic acid |
| DAG 34:1 | HMDB0007102 | DG(16:0/18:1(9Z)/0:0) |
| PE 16:1/18:2 | HMDB0008961 | PE(16:1(9Z)/18:2(9Z,12Z)) |
| PC 16:1/18:2 | HMDB0008006 | PC(16:1(9Z)/18:2(9Z,12Z)) |
| MAG 18:1 | HMDB0011567 | MG(18:1(9Z)/0:0/0:0) |
| MAG 16:0 | HMDB0011533 | MG(0:0/16:0/0:0) |
| DAG 34:2 | HMDB0007103 | DG(16:0/18:2(9Z,12Z)/0:0) |
| DAG 16:0/20:2 | HMDB0007109 | DG(16:0/20:2(11Z,14Z)/0:0) |
| DAG 18:1/18:2 | HMDB0007190 | DG(18:1(11Z)/18:2 |
| | | (9Z,12Z)/0:0) |
| LPE 18:1 | HMDB0011506 | LysoPE(18:1(9Z)/0:0) |
| TAG 16:0/ | HMDB0044553 | TG(16:0/20:4(8Z,11Z,14Z,17Z)/ |
| 20:4/20:5 | | 20:5(5Z,8Z,11Z,14Z,17Z)) |
| DAG 18:2/20:1 | HMDB0007253 | DG(18:2(9Z,12Z)/20:1 |
| | | (11Z)/0:0) |
| PC 16:1/16:1 | HMDB0008002 | PC(16:1(9Z)/16:1(9Z)) |
| PC 16:1/18:2 | HMDB0008006 | PC(16:1(9Z)/18:2(9Z,12Z)) |
| PE 16:0/16:1 | HMDB0008924 | PE(16:0/16:1(9Z)) |
| PE 16:0/18:1 | HMDB0008927 | PE(16:0/18:1(9Z)) |
| PS 34:3 | HMDB0012409 | PS(18:3(9Z,12Z,15Z)/16:0) |
| LPC 18:1 | HMDB0002815 | LysoPC(18:1(9Z)) |

### Quantification and Statistical Analysis

T-test, one-way ANOVA, and two-way ANOVA were performed in R (version 3.5.1) environment and GraphPad Prism7 with significance and confidence level 0.05 (95% confidence interval).

### Example 2

### IKE Potently Reduces DLBCL Cell Number

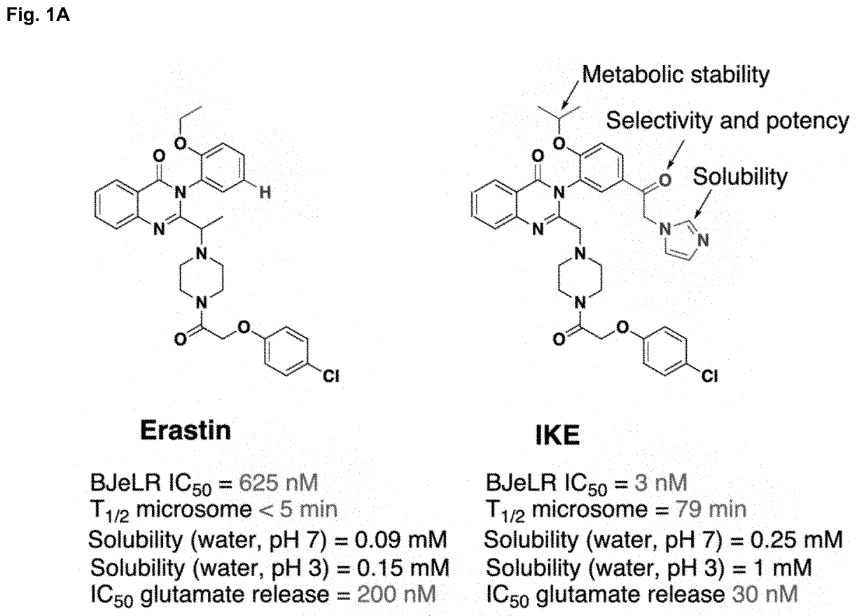

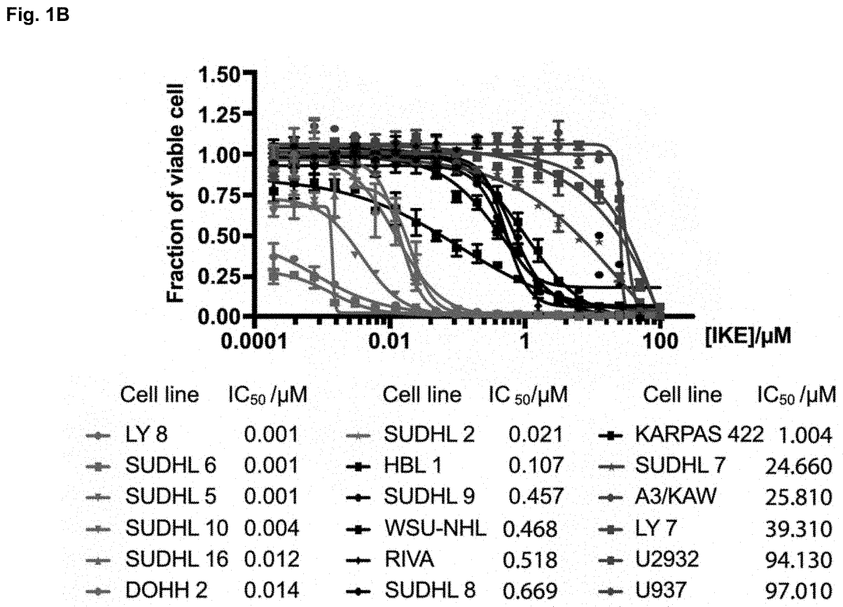

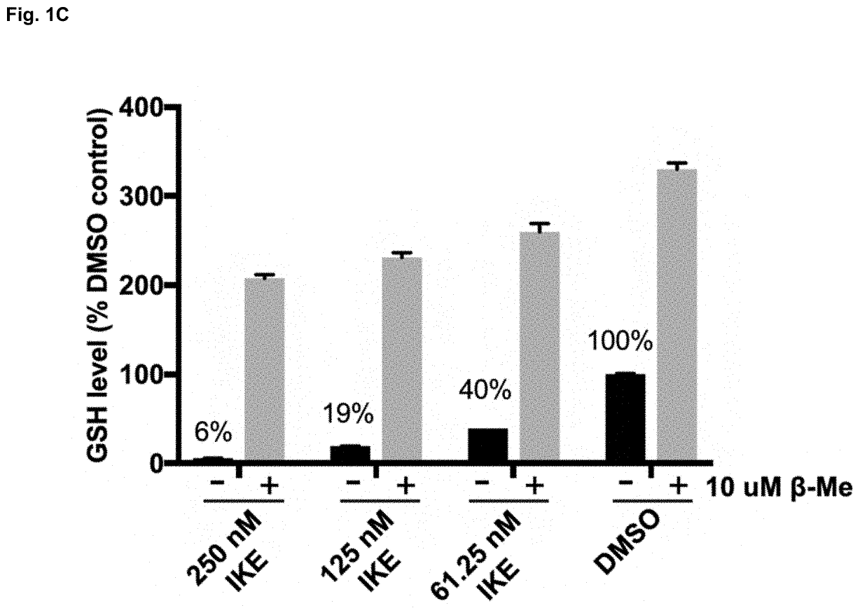

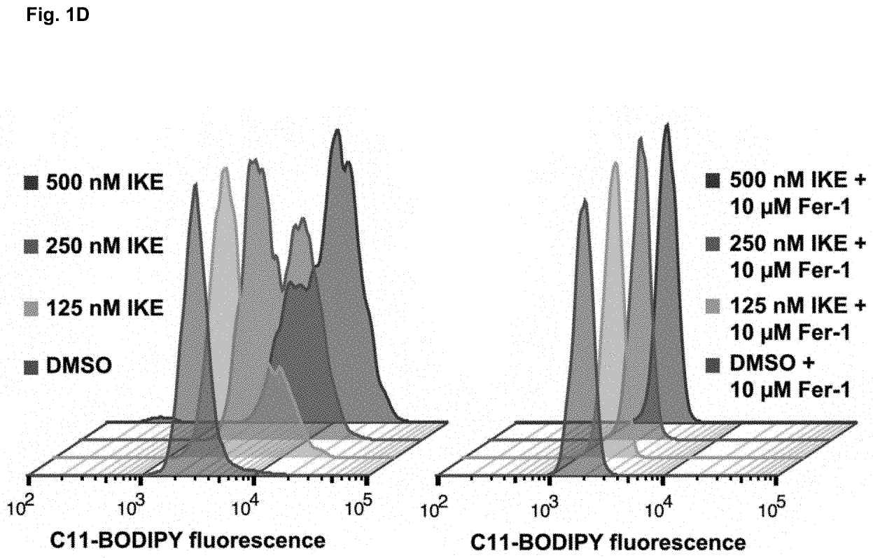

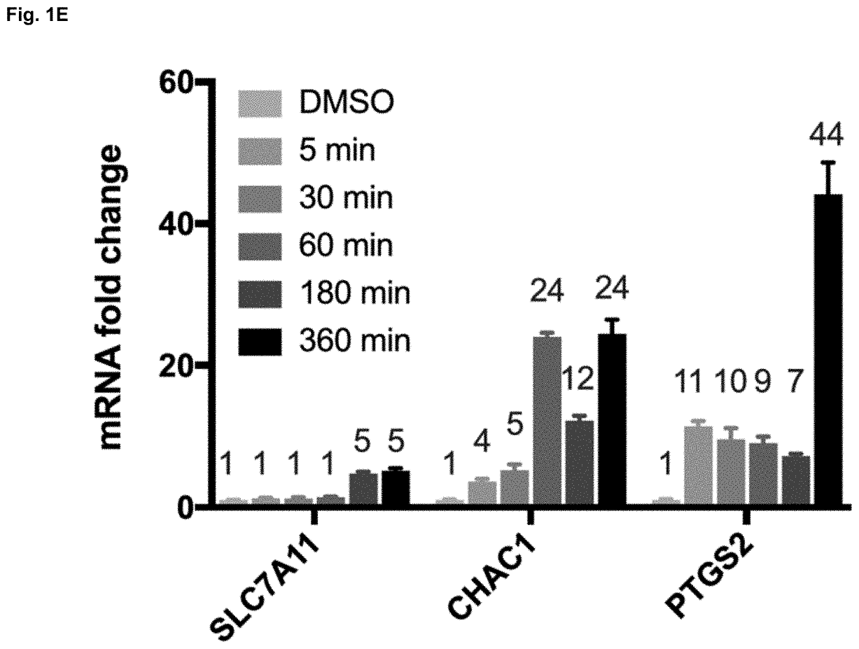

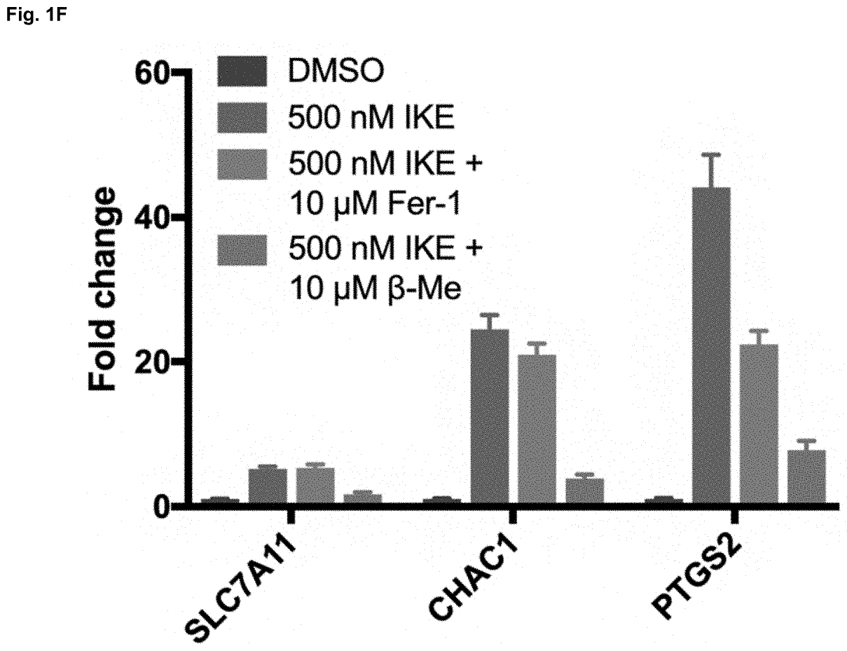

The ferroptosis inducer and system x_{c}⁻ inhibitor erastin is a useful small molecule for in vitro studies, but is metabolically labile and has low water solubility and potency, which precludes its use in vivo. The small molecule IKE is an erastin analog incorporating a metabolically stable carbonyl (FIG. 1A), which can potentially form a reversible covalent interaction with proteins, resulting in >100× potency improvement comparing to erastin in some cell lines (Yang et al. 2014; Larraufie et al. 2015). Substitution of an ethoxy moiety with isopropoxy resulted in improved metabolic stability, and the imidazole moiety in IKE helps increase water solubility and stability of the ketone and makes IKE soluble under acidic conditions.

The ferroptosis inducer and system x_{c}⁻ inhibitor erastin is a useful small molecule for in vitro applications, but it is metabolically labile and has low water solubility and potency, precluding its use in vivo. The small molecule IKE is an erastin analog incorporating a carbonyl (FIG. 1A) that can potentially form a reversible covalent interaction with proteins, resulting in >100× potency improvement compared with erastin in some cell lines (Larraufie et al., 2015). Substitution of an ethoxy moiety with isopropoxy resulted in improved metabolic stability, and the imidazole moiety in IKE helped increase water solubility and stability of the ketone, making IKE soluble under acidic conditions.

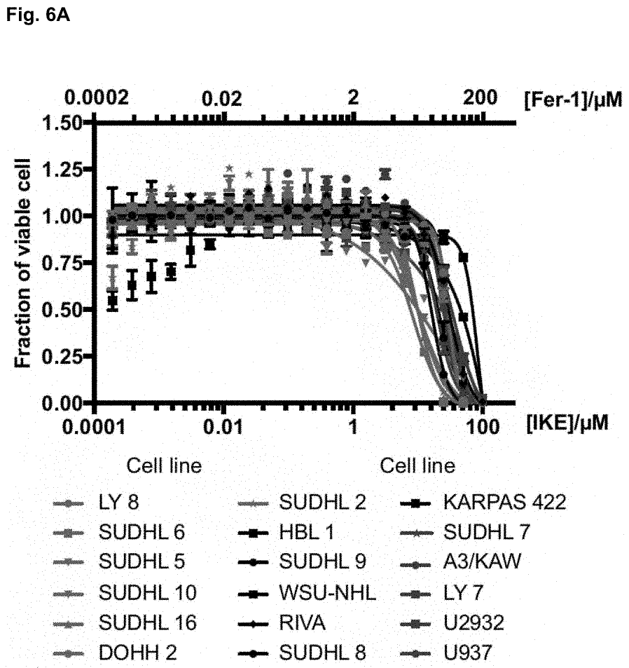

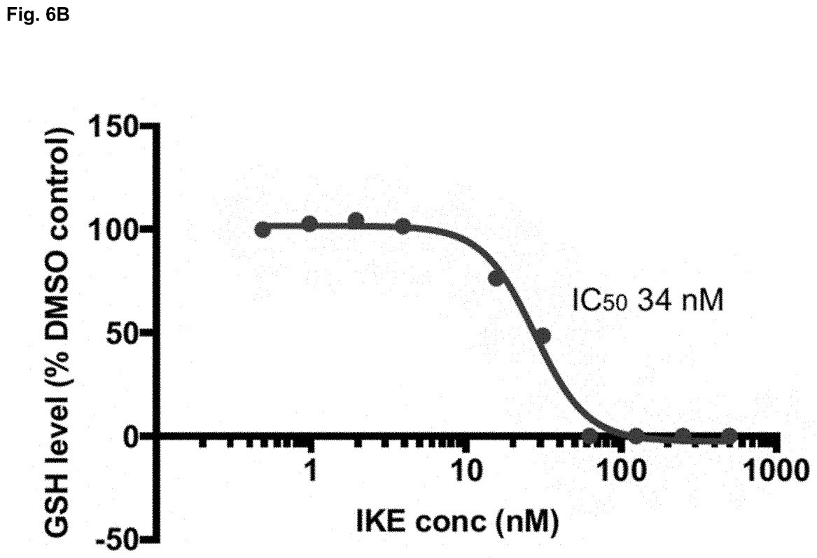

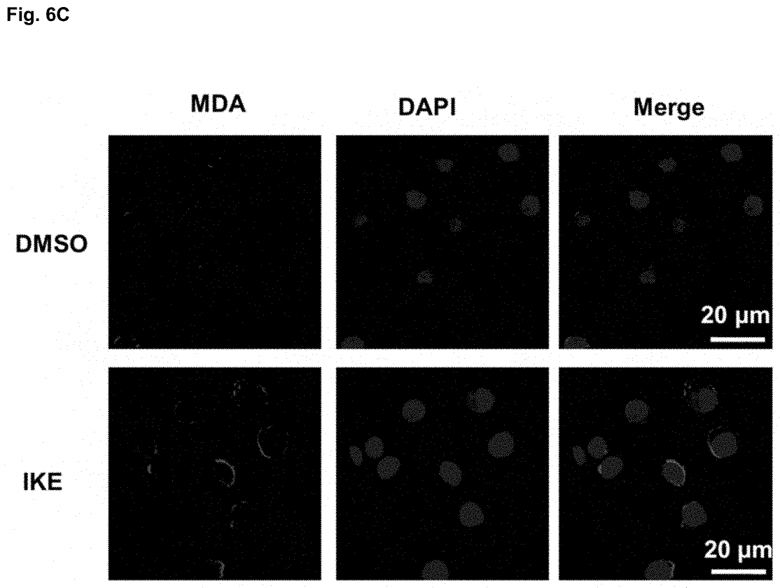

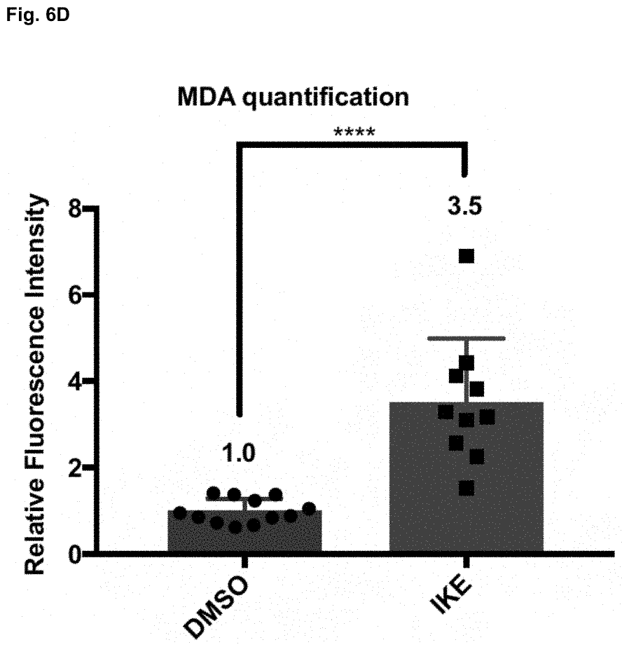

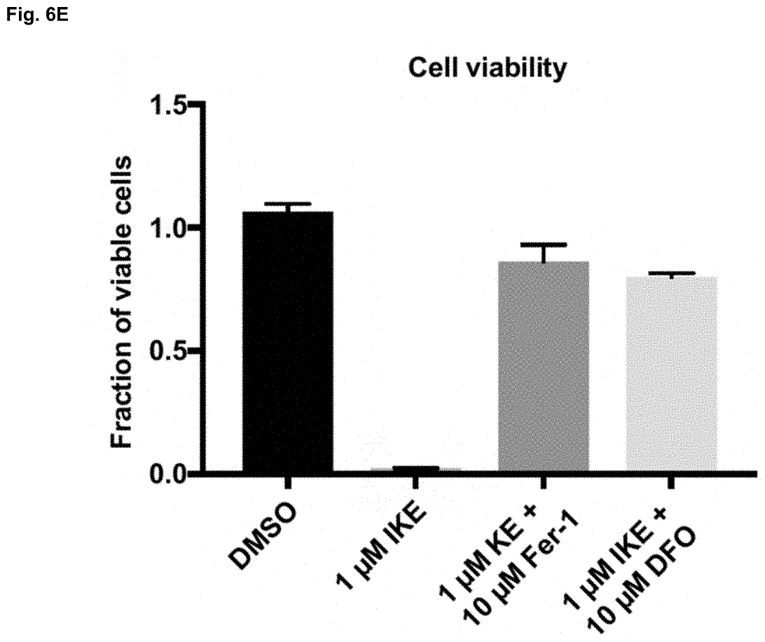

We sought to evaluate DLBCL cell line sensitivity to ferroptosis and to establish a lymphoma xenograft model using a ferroptosis-sensitive DLBCL cell line. A panel of 18 DLBCL cell lines representing distinct DLBCL subtypes, including germinal center B cell-like, activated B cell-like, and unclassified subgroups, was evaluated. The 18 DLBCL cell lines showed differential sensitivity to IKE inhibition, with cell lines exhibiting half-maximal inhibitory concentration (IC₅₀)<100 nM classified as sensitive cell lines, those with IC₅₀>10 μM classified as resistant cell lines, and those with IC₅₀ values between 100 nM and 10 μM classified as having intermediate resistance (FIG. 1B). We further tested the degree of IKE-induced lethality upon co-treatment with the ferroptosis inhibitor fer-1, a radical-trapping antioxidant that inhibits lethal lipid peroxidation during ferroptosis (Skouta et al., 2014; Zilka et al., 2017). Co-treatment with fer-1 rescued cell death induced by IKE in DLBCL cell lines, indicating that IKE-induced lethality in these cell lines resulted from lipid peroxidation and ferroptosis (FIG. 6A). Among the sensitive DLBCL cell lines, we selected SUDHL6 for generating a subcutaneous xenograft model in 6-week-old male NCG mice.

### Example 3

### IKE Pharmacodynamic (PD) Study In Vitro

We aimed to investigate whether IKE specifically inhibited system x_{c}⁻ and induced ferroptosis in DLBCL cells. Previous studies found that IKE inhibited glutamate release, and the IKE parental analog erastin inhibited cysteine uptake. Thus, we tested the cellular level of reduced GSH, which requires cysteine for its biosynthesis, as a readout of IKE potency. A fluorometric method revealed dose-dependent GSH depletion by IKE (FIG. 1C); this effect was reversed by co-treatment with 10 μM β-ME, which reduces cysteine to cysteine, allowing its import into cells through systems A, ASC, and L, thus circumventing inhibition of system x_{c}⁻. The IC₅₀ of GSH depletion by IKE was 34 nM (FIG. 6B) in SUDHL6 cells, while sulfasalazine's IC₅₀ for GSH depletion is in the millimolar range (Narang et al., 2007; Lo et al., 2010).

We next sought to evaluate whether IKE treatment causes lipid peroxidation, a marker of ferroptosis, in DLBCL cells. Analysis of lipid reactive oxygen species (ROS) by flow cytometry using the lipid peroxidation probe C11-BODIPY revealed a dose-dependent increase in lipid ROS upon IKE treatment in SUDHL6 cells (FIG. 1D). Co-treatment with 10 μM fer-1 inhibited this signal, as expected (FIG. 1D). We reasoned that lipo-oxidative stress markers, including lipid-peroxide-product-derived protein modifications might also be used as ferroptosis biomarkers. Malondialdehyde (MDA) is a naturally occurring reactive carbonyl compound that is derived from lipid peroxidation of PUFAs. Immunofluorescence staining with an anti-dihydropyridine-MDA-lysine adduct antibody (mAb 1F83) (Yamada et al., 2001) revealed that dihydropyridine-MDA-lysine adduct abundance was increased upon IKE treatment (FIGS. 6C and 6D).

To facilitate molecular characterization of ferroptosis induction in DLBCL cells, we analyzed gene expression biomarkers of ferroptosis in these cell lines by RT-qPCR. Expression of genes involved in ferroptosis (Stockwell et al., 2017) was analyzed upon IKE treatment. GPX4, cystathionine b-synthase (CBS), and ACSL4 were not upregulated with IKE treatment (data not shown). However, the system x_{c}⁻ component SLC7A11, prostaglandin-endoperoxide synthase 2 (PTGS2, which encodes cyclooxygenase-2), and ChaC GSH-specific γ-glutamylcyclotransferase 1 (CHAC1) expression was significantly increased in SUDHL6 cells following IKE treatment (FIG. 1E). Compared with the upregulation of PTGS2 and CHAC1, induction of SLC7A11 expression requires a relatively longer-term IKE treatment. PTGS2 was reported to be upregulated by the lipid peroxidation product 4-hydroxy-2-nonenal (Uchida, 2017) and by GPX4 depletion (Sengupta et al., 2013). Co-treatment with fer-1 inhibited PTGS2 upregulation, suggesting that PTGS2 is a functional biomarker of ferroptosis. Fer-1 co-treatment did not inhibit CHAC1 upregulation, as expected, indicating that CHAC1 is a parallel downstream marker of system x_{c}⁻ inhibition not affected by lipid peroxidation (FIG. 1F). In addition, co-treatment with 10 μM β-ME prevented IKE-induced upregulation of SLC7A11, PTGS2, and CHAC1, suggesting that the upregulation of these genes is downstream of cysteine starvation.

### Example 4

### Untargeted Lipidomics of IKE-Treated DLBCL Cells

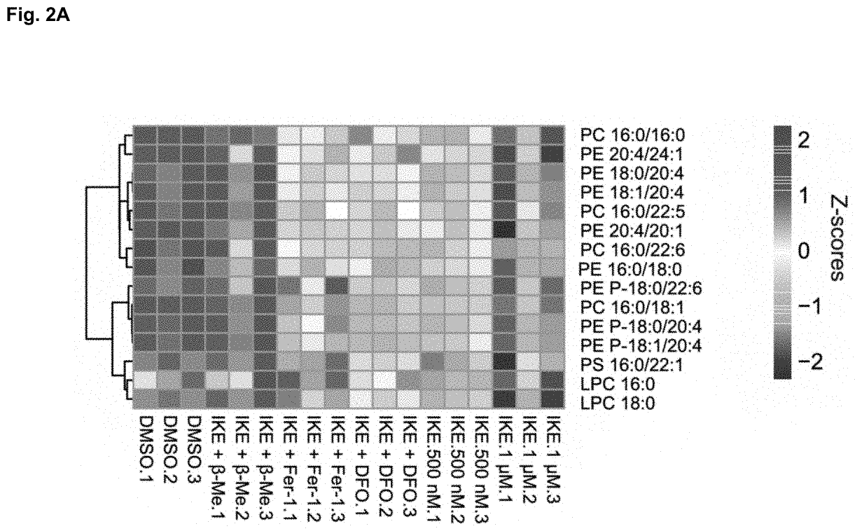

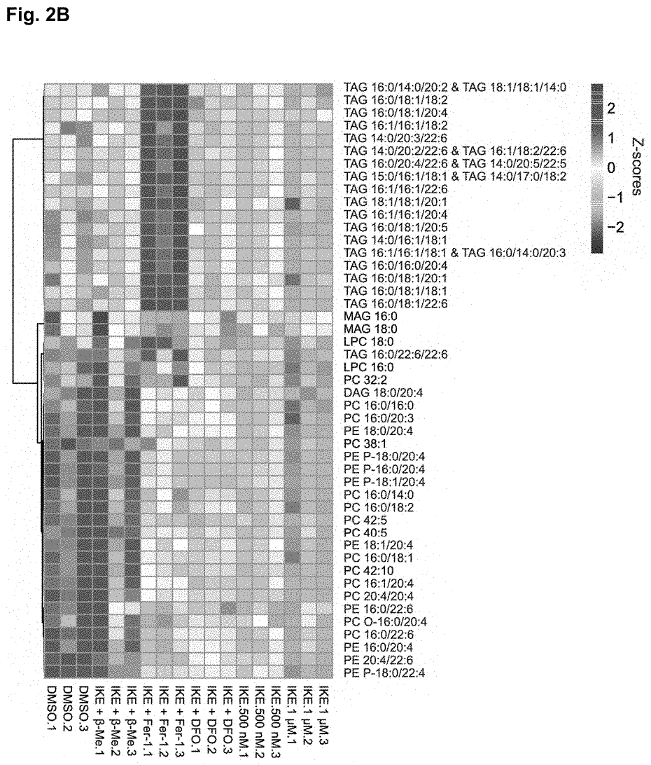

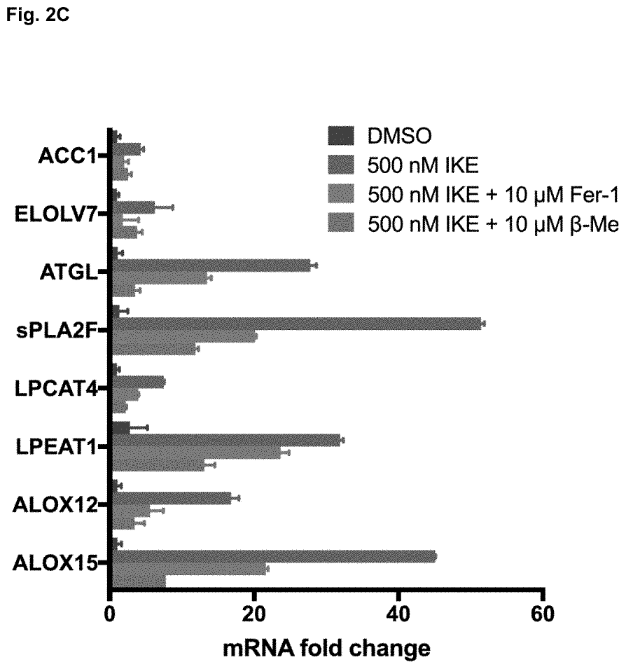

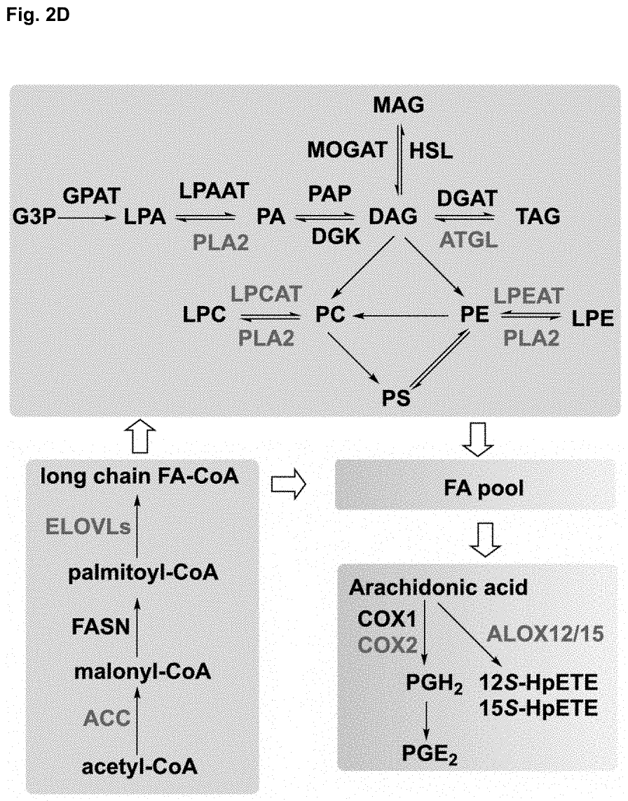

To investigate the effects of IKE on lipid composition and metabolites, we performed untargeted MS-based lipidomics and gene expression analysis of related enzymes during lipid biosynthesis and oxidation. SUDHL6 cells treated with 1 mM IKE, and 500 nM IKE with or without 10 μM fer-1, 10 μM β-Me, or 10 μM deferoxamine (DFO) co-treatment, were subjected to UPLC-MS analysis. The annotations of the lipid species were determined with Lipostar software (v.1.0.4, Molecular Discovery, UK) (Goracci et al., 2017). We observed significant (one-way ANOVA, p<0.05) alterations in the relative abundance of 62 lipid species, including lysophosphatidylcholines (LPCs), phosphatidylcholines (PCs), phosphatidylethanolamines (PEs), and TAGs mainly containing PUFAs, in IKE-treated samples, while co-treatment with β-ME reversed these effects and maintained the levels of these specific lipids to near the amounts on vehicle-treated cells (FIGS. 2A and 2B). The decrease in the phospholipids PC and PE upon IKE treatment is consistent with the effects of piperazine-erastin treatment and erastin treatment on lipid composition in HT-1080 cells (Yang et al., 2016; Skouta et al., 2014). The decrease in TAG lipids upon IKE treatment indicates TAG lipids may be susceptible to oxidation during ferroptosis. Fer-1 cotreatment significantly increased TAG lipids and decreased MAG lipids, suggesting a possible protective role of TAG as a buffer against oxidative damage (Listenberger et al., 2003). With the induction of oxidants and oxidative stress, lipids can be oxidized via both iron-mediated and enzyme-mediated lipid peroxidation. While co-treatment with DFO inhibited IKE induced cell death in culture (FIG. 6E), it only partially eliminated lipidomic changes upon IKE treatment, possibly due to DFO inhibiting iron-mediated lipid peroxidation, but not enzyme-mediated lipid peroxidation, suggesting that only a subset of lipidomic changes are needed for inducing cell death.

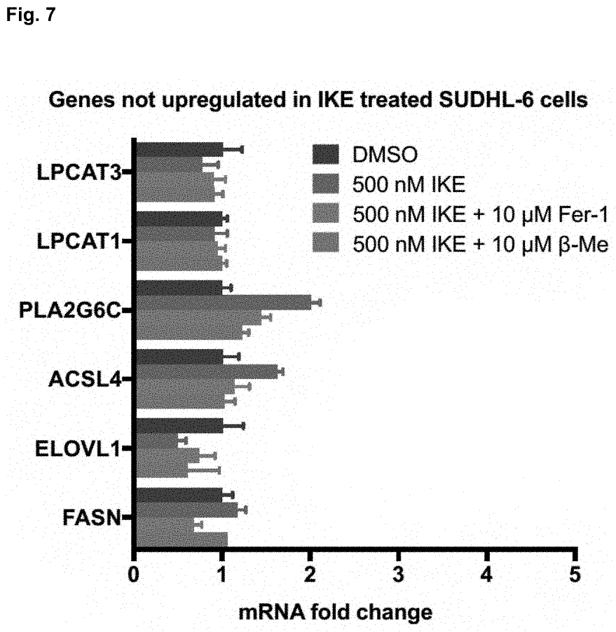

By measuring the mRNA levels of lipid biosynthesis enzymes upon IKE treatment, we discovered that de novo lipid biosynthesis pathways, phospholipid remodeling pathways, and arachidonic acid oxidation pathways were activated (FIG. 2C). First, there was significant upregulation of ACC1, which catalyzes the rate-limiting step of fatty acid biosynthesis from acetyl-CoA to malonyl-CoA, and ELOVL7, which catalyzes the rate-limiting reaction of long-chain fatty acid elongation, especially the elongation of C18:3 (n-3) and C18:3 (n-6)-CoAs (FIGS. 2C and 7). Second, lipid peroxidation during ferroptosis is a deleterious process, and one way to repair such damage is to remodel lipids, by selectively cleaving the oxidized PUFA tail, replacing it with non-oxidized fatty acids, and subsequently reducing lipid hydroperoxides with GSH peroxidase (Chakraborti, 2003). The activation of the phospholipid remodeling pathway, including sPLA2, which selectively releases PUFAs at the sn-2 position of phospholipids; ATGL, which hydrolyzes TAGs; and LPCAT4 and LPEAT1, which catalyze the conversion of LPC to PC, and LPE to PE, respectively, is thus important for the oxidative-damage-repair process. Third, in addition to Fenton chemistry-mediated lipid peroxidation, enzyme-mediated lipid peroxidation was also activated in IKE-induced ferroptosis, as there was increased expression of lipoxygenases ALOX12 and ALOX15. Genes upregulated following IKE treatment are highlighted in red in the schematic lipid metabolism overview (FIG. 2D). The upregulation of these genes was partially reversed by co-treatment of 10 μM fer-1 or 10 μM β-ME (FIG. 2C), indicating that the observed changes in gene expression result from cysteine depletion and oxidative stress induced by IKE. In summary, these lipidomic changes provide us with a detailed picture of IKE-induced ferroptosis in cell culture at the level of lipid metabolism.

### Example 5

### IKE Pharmacokinetics (PK) and Pharmacodynamics (PD) In Vivo

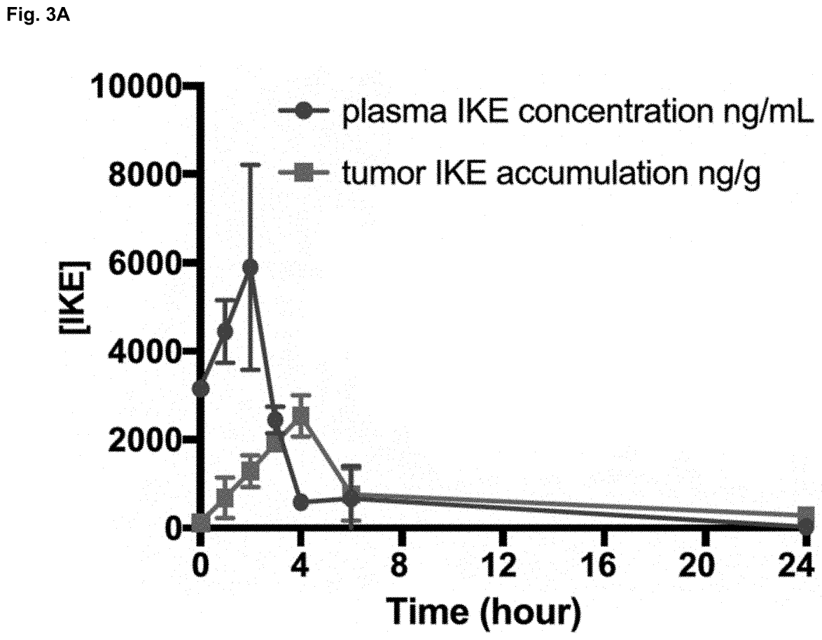

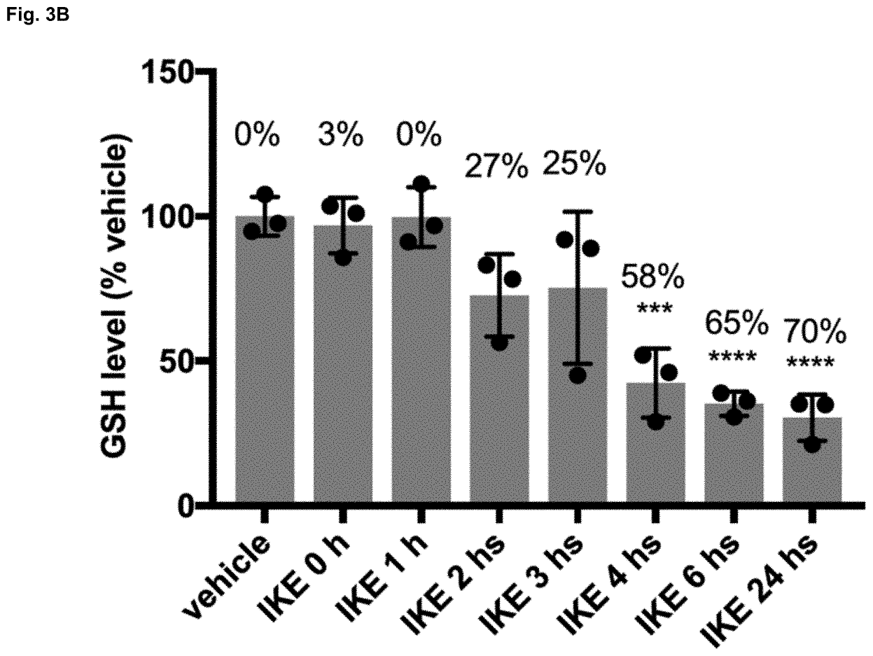

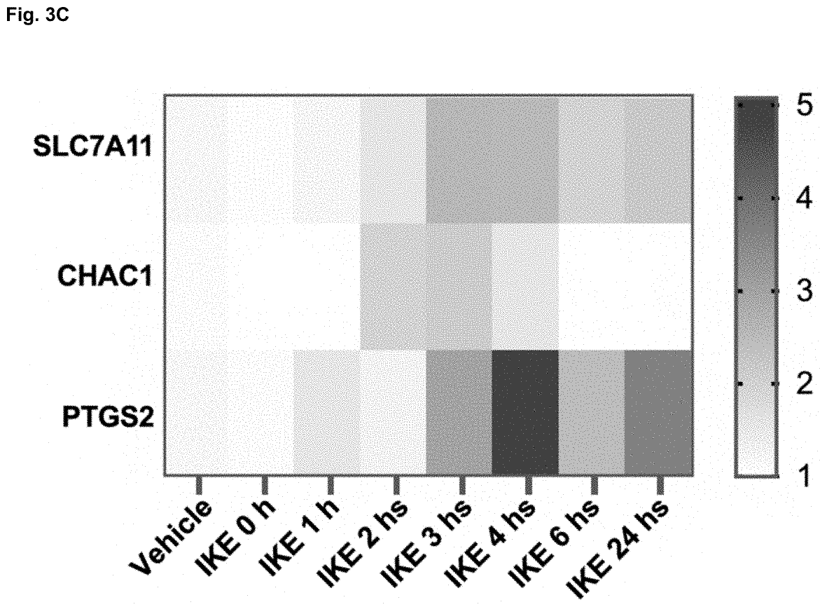

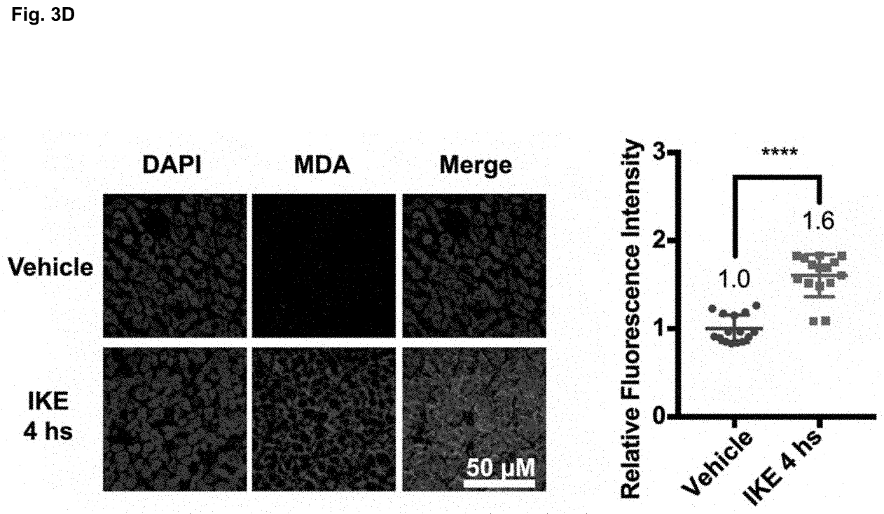

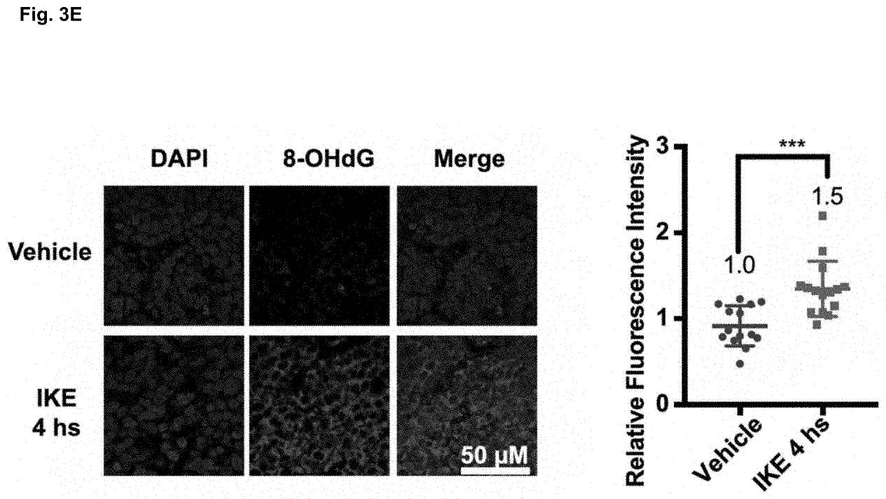

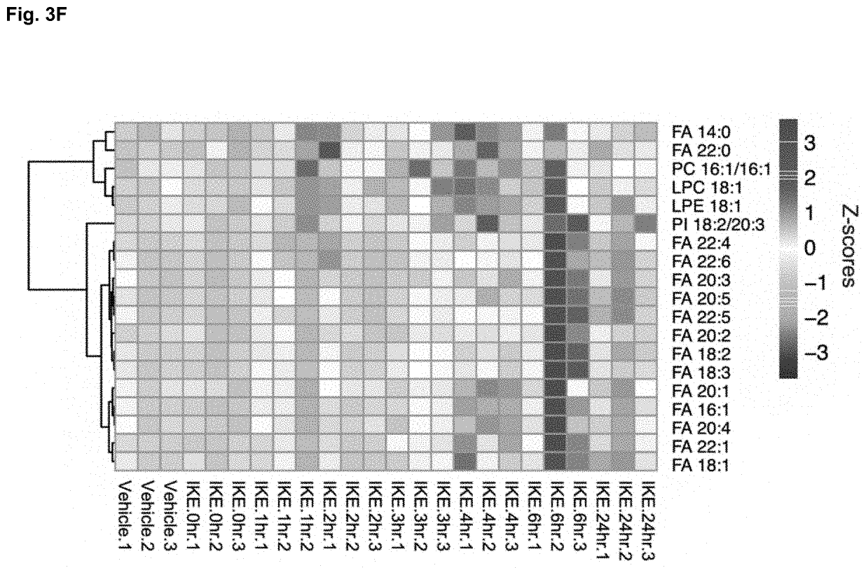

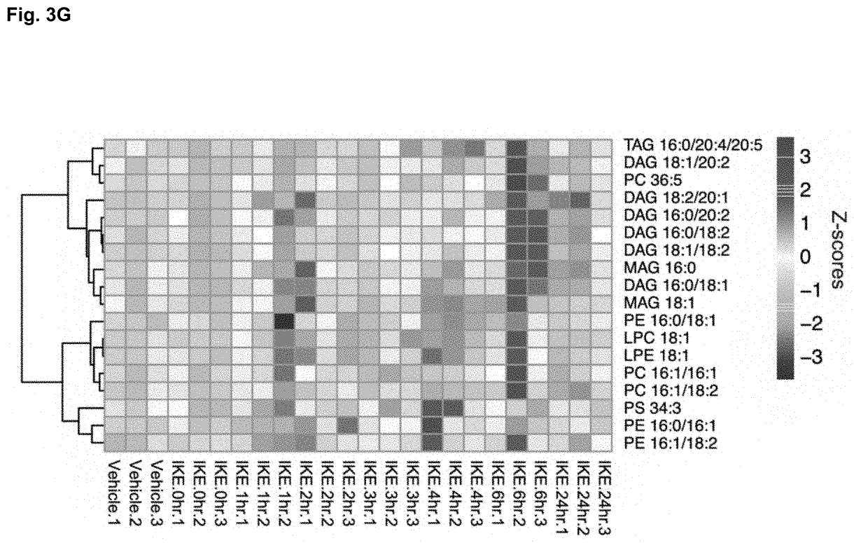

To determine the suitability of IKE for in vivo studies, we first evaluated multiple dosage routes by administering a single dose of IKE (50 mg/kg, 5% DMSO in Hank's balanced salt solution [HBSS] at pH 4) using intraperitoneal (i.p.), intravenous, and oral routes in non-obese diabetic/severe combined immunodeficiency (NOD/SCID) mice. Determination of IKE concentration over a period of 8 h revealed i.p. to be the most effective and practical means of IKE administration (Table 1). Next, IKE concentration in plasma and tumor samples was determined after a single dose of IKE (50 mg/kg, 5% DMSO in HBSS at pH 4, i.p.) in SUDHL6-xenograft-bearing NCG mice over a period of 24 h. IKE reached the highest plasma concentration of 5.2 μg/mL at 1.35 h, and the highest tumor accumulation of 2.5 μg/mL at 3.30 h (FIG. 3A, Table 2).

We collected tumor samples from each time point after a single dose of IKE (50 mg/kg, 5% DMSO in HBSS) and evaluated the abundance of ferroptosis markers. There was significant GSH depletion upon IKE dosing starting from 4 h (FIG. 3B). The GSH depletion in tumors induced by IKE was persistent at 24 h, even when there was little IKE left in the bulk tumor sample. Consistent with IKE accumulation in the tumor samples, CHAC1, SLC7A11, and PTGS2 mRNAs were upregulated starting at 3 h (3- to 5-fold for PTGS2 and SLC7A11, and 2-fold for CHAC1) (FIG. 3C). Immunofluorescence analysis of samples at 4 h post-treatment showed that there was increased abundance of a hydropyridine-MDA-lysine adduct and 8-hydroxy-2′-deoxyguanosine (8-OHdG), biomarkers for oxidative lesions, indicating that IKE induced lipid peroxidation and more general oxidative stress in these tumor samples (FIGS. 3D-3E).

### Example 6

### IKE Untargeted Lipidomic Study In Vivo

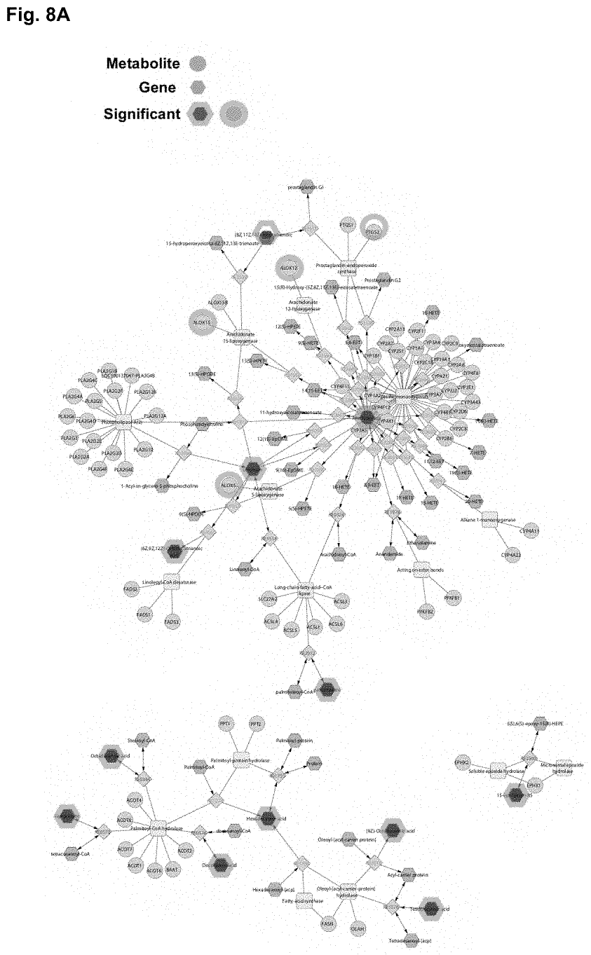

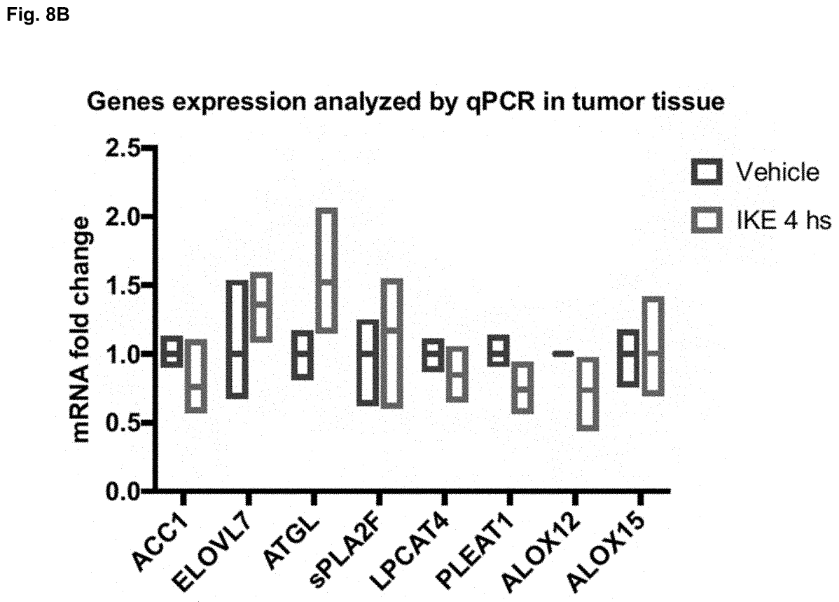

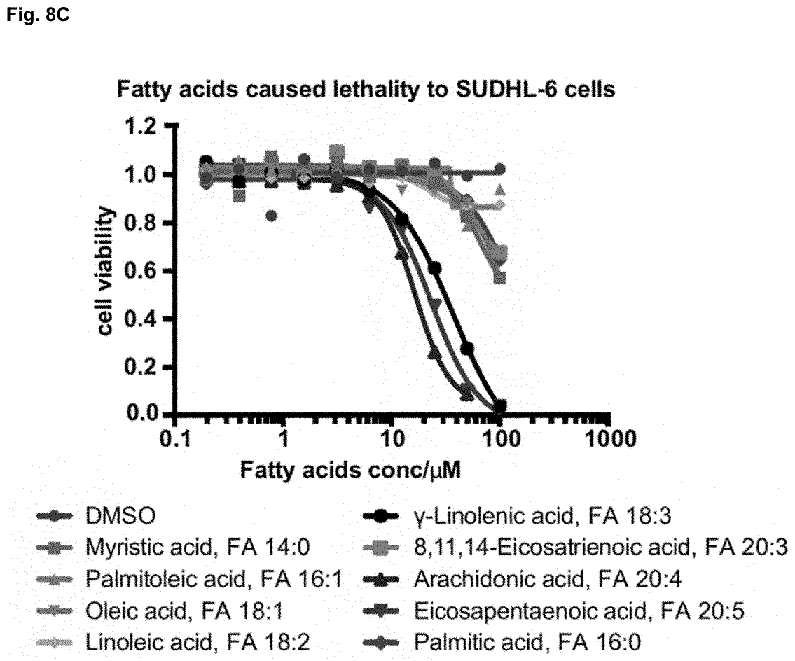

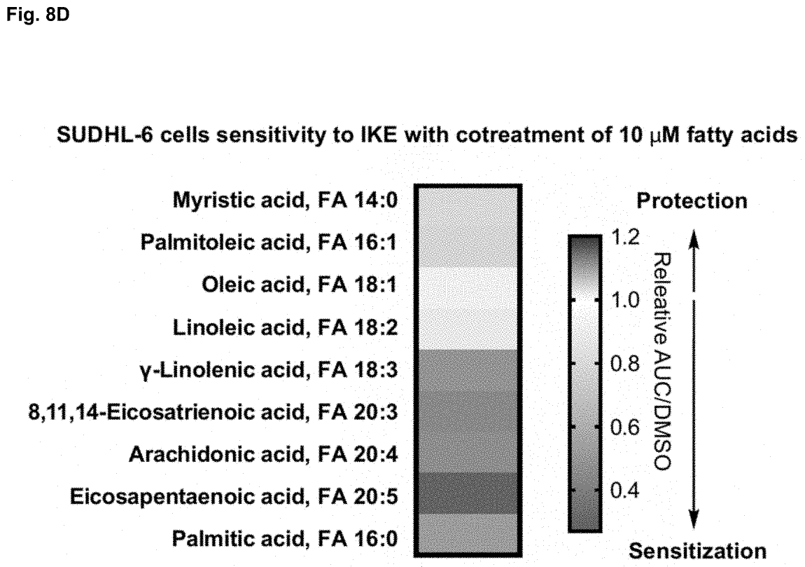

We sought to investigate lipidomic changes caused by IKE treatment in vivo. We performed untargeted lipidomics on tumor tissue with a single dose of IKE across different time points. We identified significant (one-way ANOVA, p<0.05) increases in the relative abundance of free fatty acids, phospholipids, and diacylglycerides (DAGs) upon IKE treatment (FIGS. 3F and 3G). Differences with the cell culture experiment might stem from the different tumor microenvironment in vivo. The lipids identified were enriched in linoleic acid and arachidonic acid metabolism (FIG. 8A). The significant increase in the levels of DAGs and free fatty acids may result from ATGL-mediated TAG hydrolysis (FIG. 8B). The increased fatty acids might in turn promote phospholipid remodeling to synthesize specific phospholipids, including PCs and PEs. To explore the free fatty acids effects on cells and ferroptosis, we performed a cell survival test of free fatty acids in the presence or absence of IKE. We found that PUFAs, including γ-linolenic acid, eicosapentaenoic acid, and arachidonic acid, were toxic to cells at high concentration (100 μM) (FIG. 8C). Most free fatty acids (10 μM) sensitized cells to ferroptosis, while oleic acid and palmitoleic acid (10 μM) protected against ferroptosis, which might be due to the fact that these two fatty acids lack bis-allylic sites, which are the sites for lipid peroxidation initiation (Gaschler and Stockwell, 2017) (FIG. 8D).

### Example 7

### IKE PEG-PLGA NPs have Suitable Properties for Application In Vivo

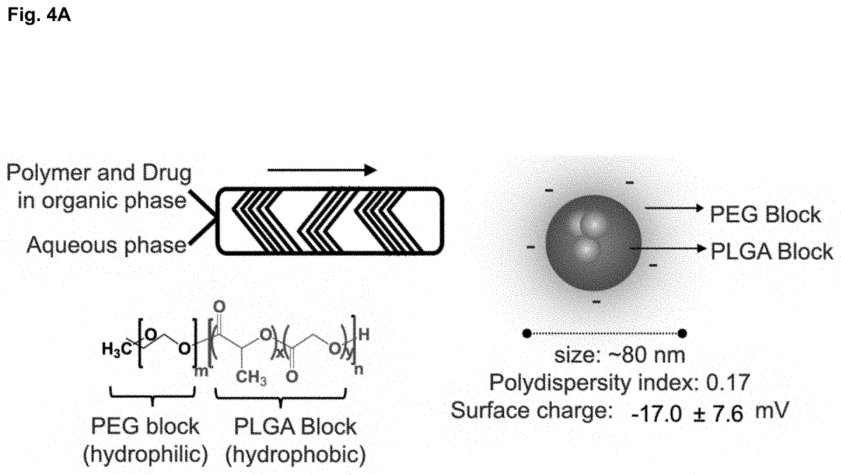

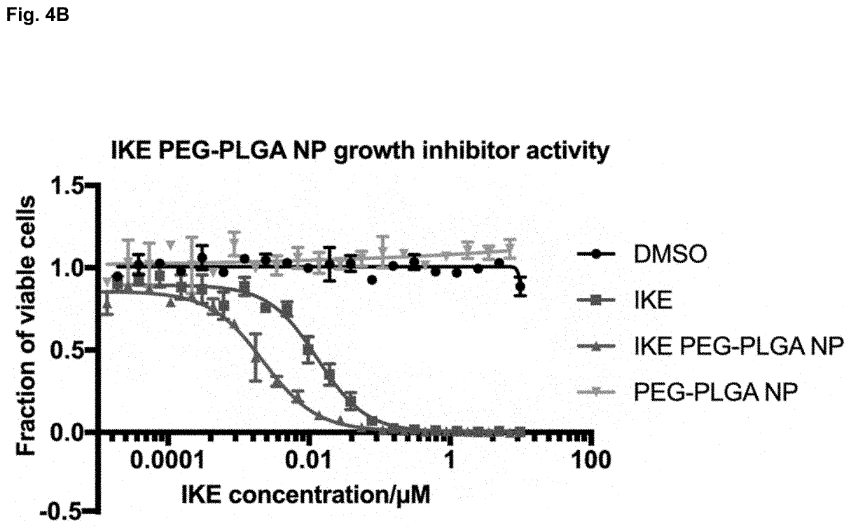

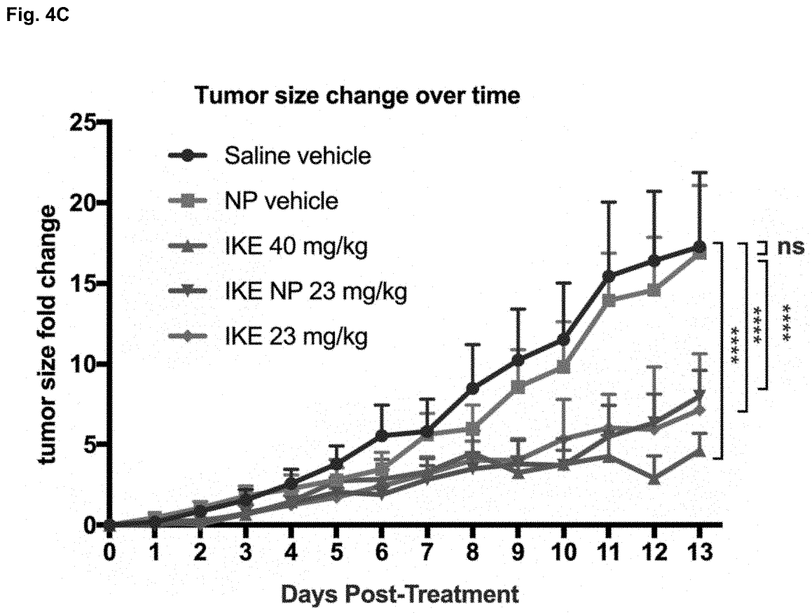

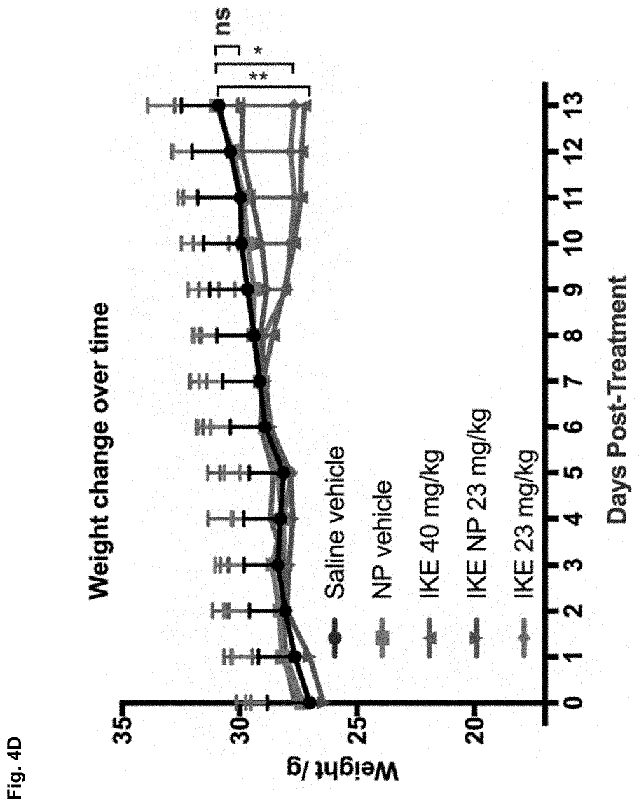

IKE is soluble under acidic aqueous conditions, but not to the same degree under neutral aqueous conditions (FIG. 1A). To improve delivery of the compound, we sought to use an NP formulation. We selected biocompatible and biodegradable PEG-PLGA di-block copolymer-based NPs as an IKE carrier system (FIG. 4A). The PEG block was used to create a deformable hydrating layer by tight associations with water molecules, which prevents clearance by the mononuclear phagocyte system, prolonging circulation lifetime. The PLGA block was used to form a hydrophobic core to incorporate IKE, which provides sustained release by diffusion and surface and bulk erosion.

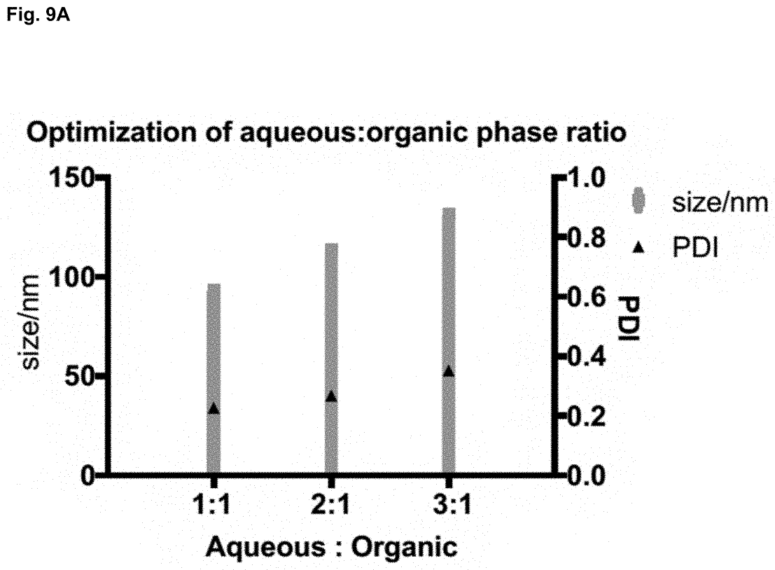

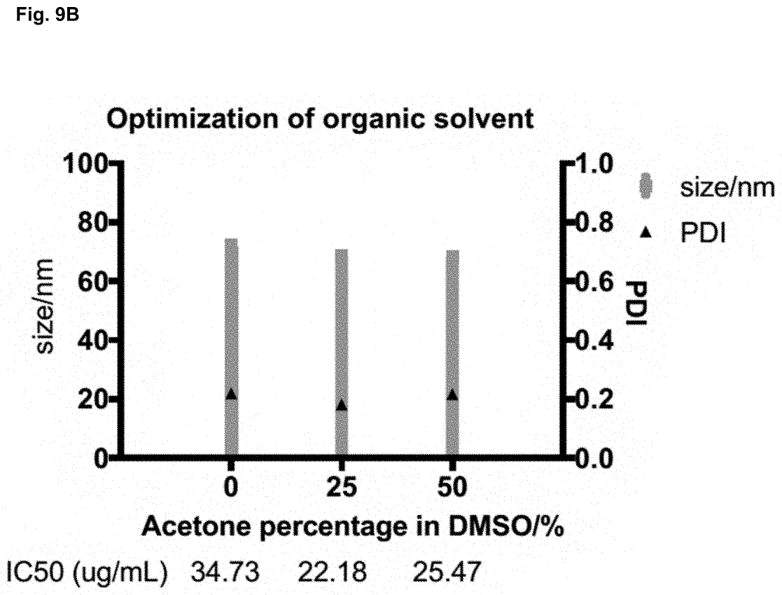

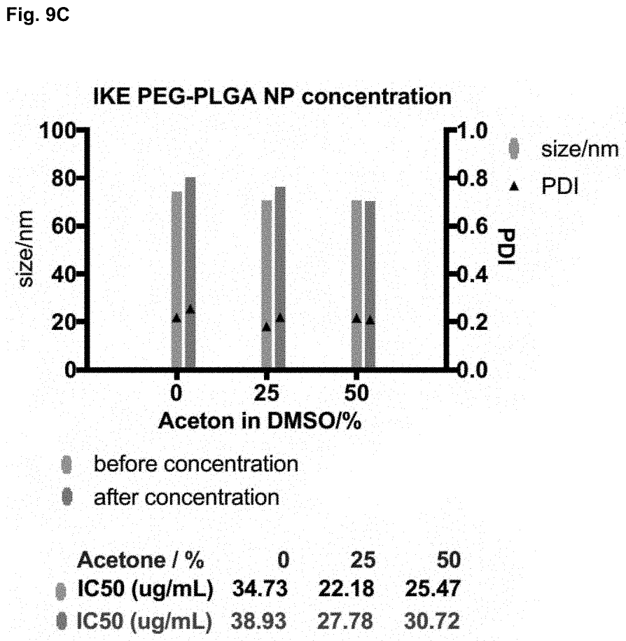

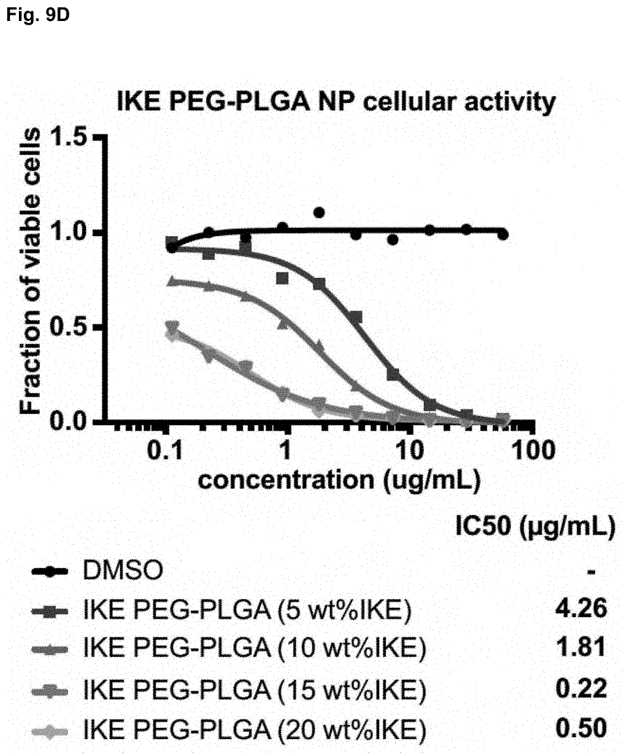

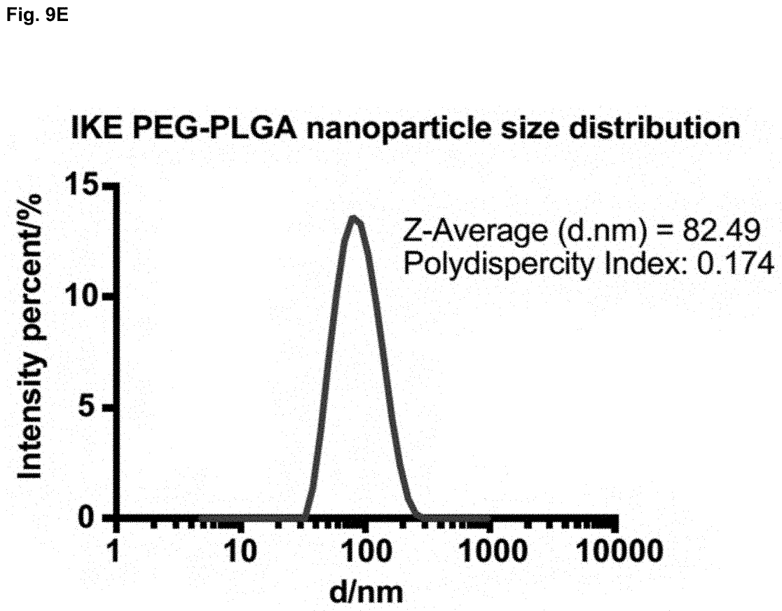

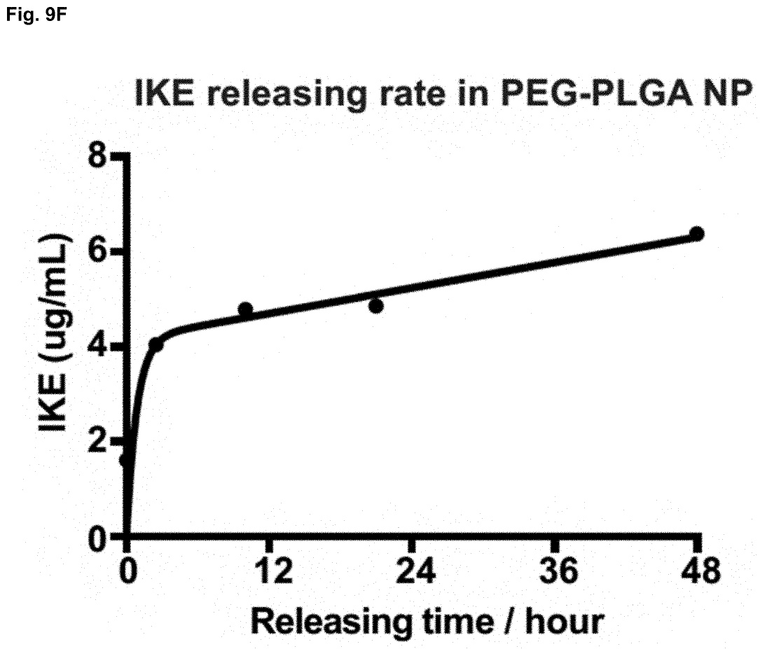

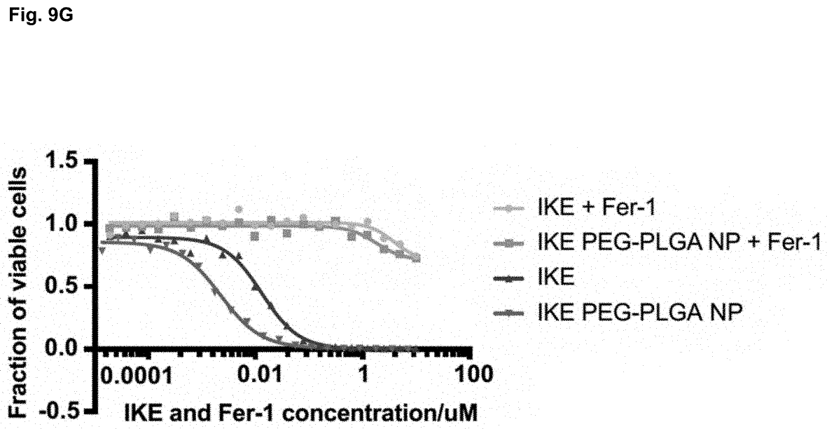

To formulate <100 nm NPs with a reproducible method, we employed the high-flow microfluidic system NanoAssemblr (Gdowski et al., 2018; Valencia et al., 2013). Self-assembly of NPs by nanoprecipitation occurs when the organic phase containing IKE and the PEG-PLGA polymer combines with the aqueous phase in a microchannel. IKE PEG-PLGA NPs with a diameter as small as 80 nm and polydispersity index of 0.17 were formulated using this approach (FIGS. 9A-9G). The surface possessed a slightly negative charge with ζ potential of −17.0 mV to prevent NP aggregation. One of the problems with the use of NPs as drug carriers in vivo is the low content of drug and low concentration of NPs in suspension. We were able to achieve concentration factors up to 20 using centrifugal filter units without causing NP aggregation or dramatically decreasing IKE loading efficiency (FIG. 9C). As a result, IKE PEG-PLGA NPs containing IKE as high as 3 mg/mL (drug loading 3.65% by weight and encapsulation efficiency of 24%) were formulated. Compared with free IKE, IKE PEG-PLGA NPs had enhanced cellular activity in SUDHL6 cells (FIG. 4B), which might result from increased internalization of IKE NPs into the cells. Co-treatment with fer-1 rescued cell death induced by IKE and IKE PEG-PLGA NPs (FIG. 9G). In addition, administration of 750 mg/kg naked PEG-PLGA NPs daily for 2 weeks did not result in any detectable weight loss or other observable signs of toxicity, suggesting that this NP system was suitable for in vivo drug delivery.

### Example 8

### IKE Inhibits Tumor Growth In Vivo and the PEG-PLGA NP Formulation Enhances its Therapeutic Index

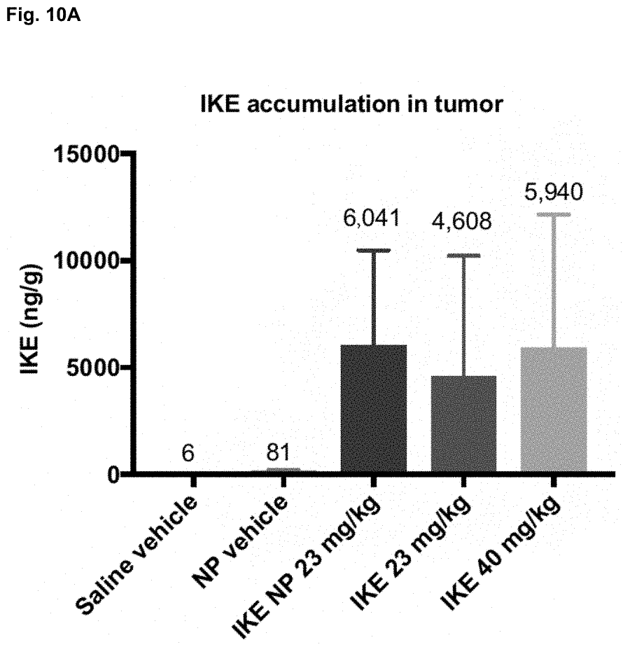

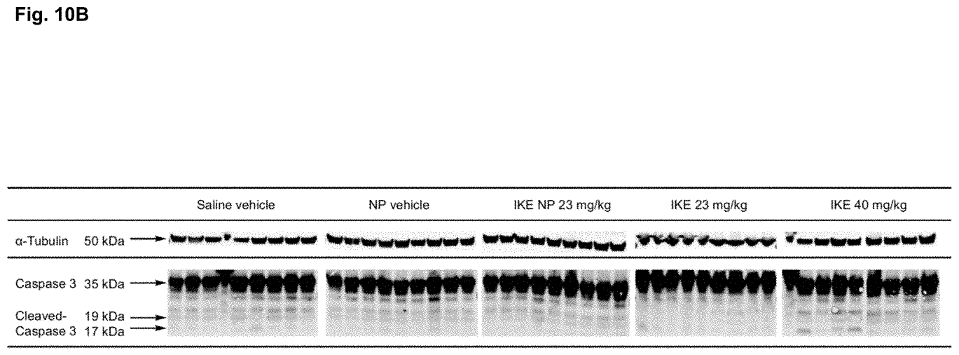

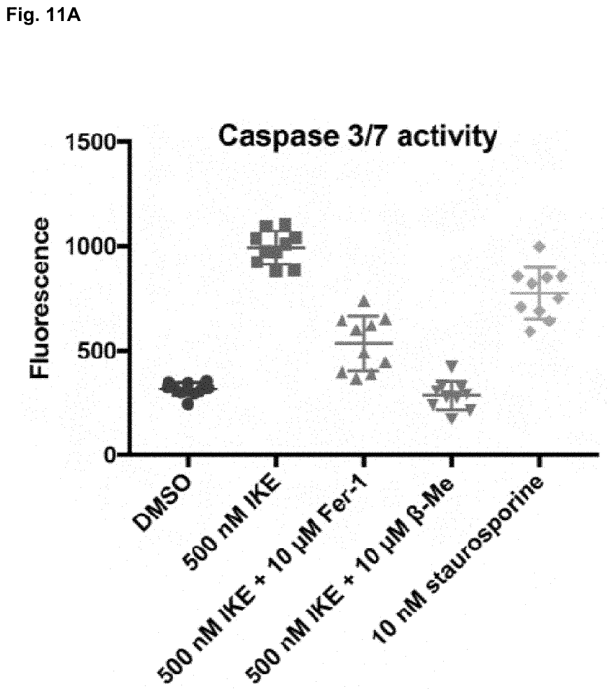

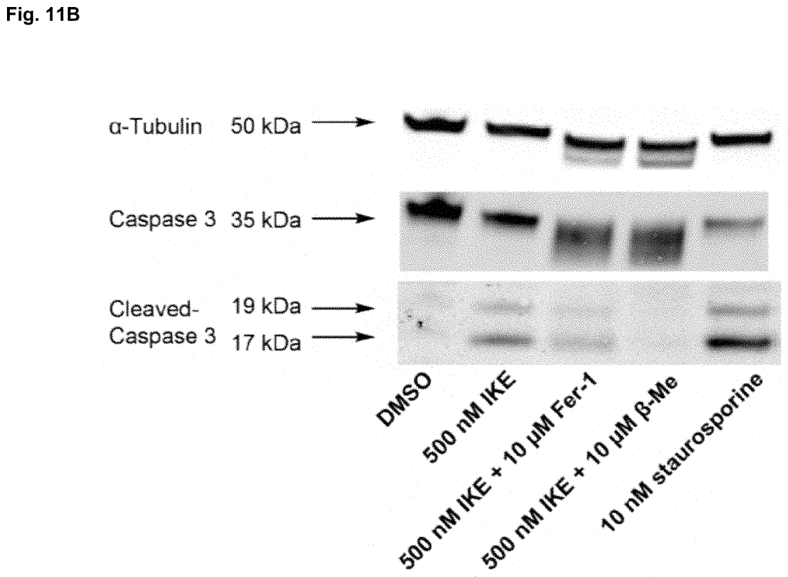

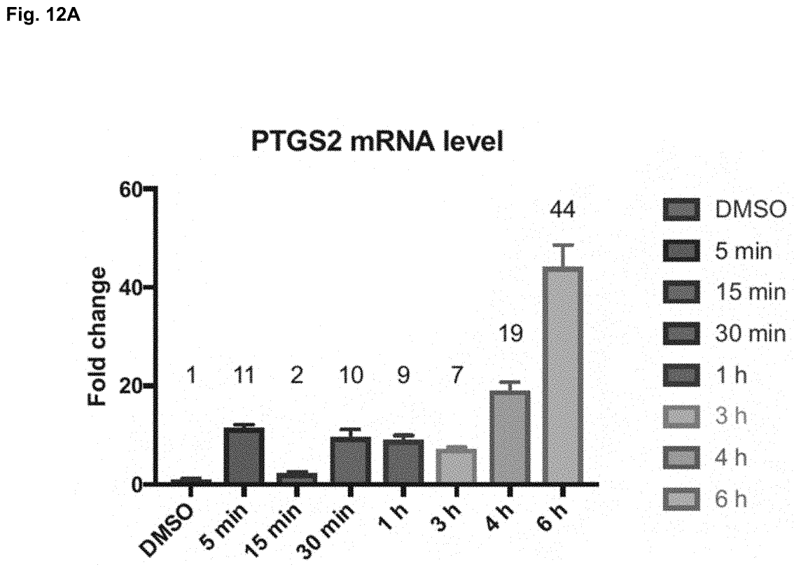

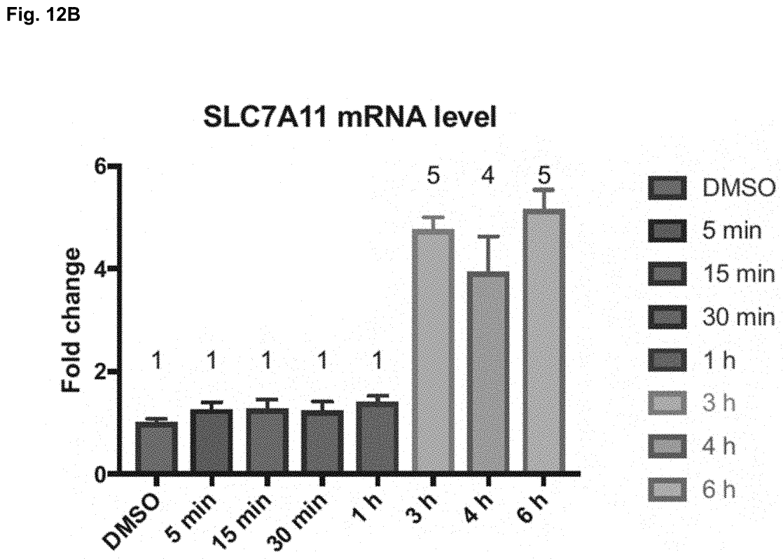

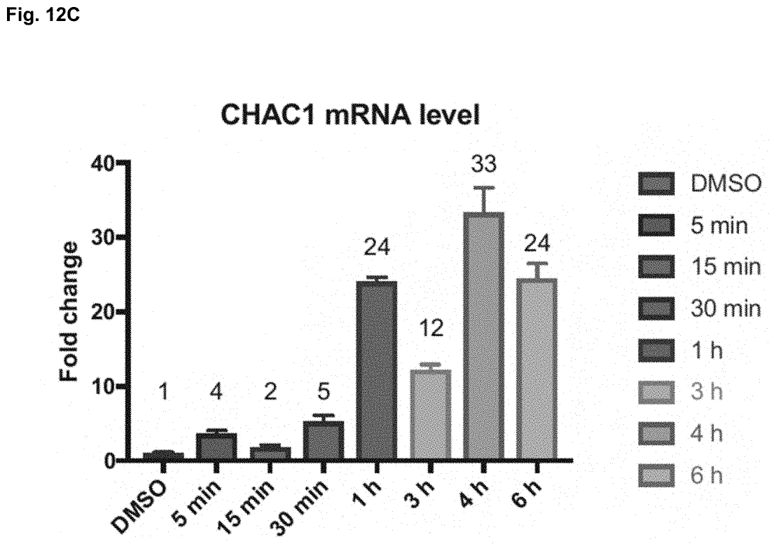

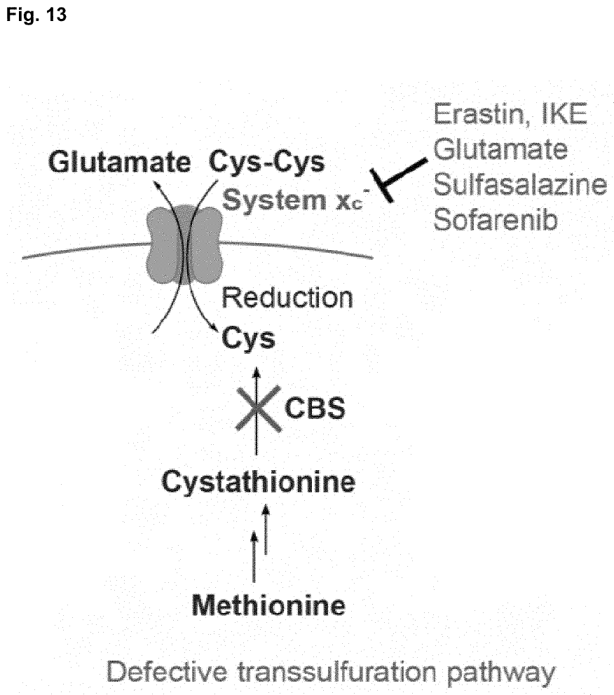

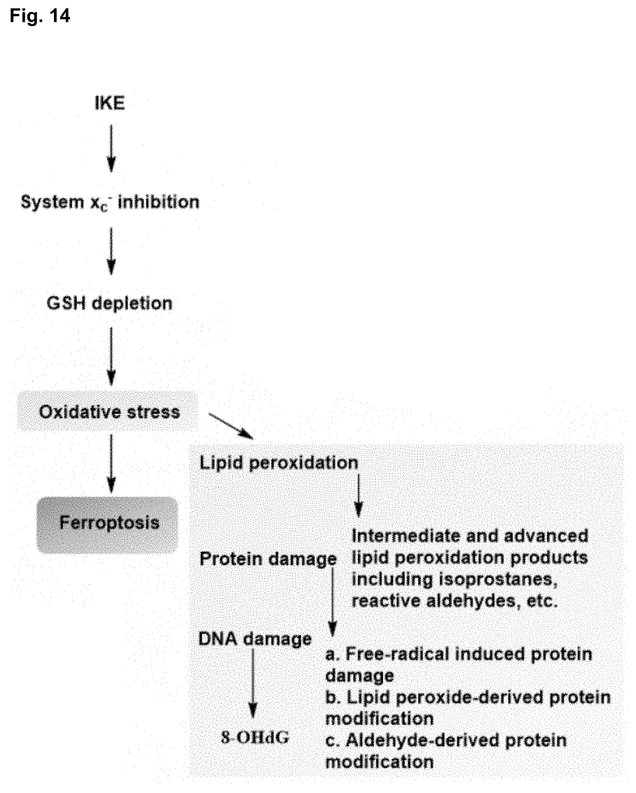

We investigated the efficacy of IKE in vivo in male NCG mice bearing SUDHL6 subcutaneous xenografts. Once tumor volumes reached 100 mm³, mice were randomized into five groups and treated with vehicle (5% DMSO in HBSS at pH 4), unfunctionalized PEG-PLGA NPs in water, 40 mg/kg free IKE (5% DMSO in HBSS at pH 4), 23 mg/kg free IKE (5% DMSO in HBSS at pH 4), or 23 mg/kg IKE NPs (IKE PEG-PLGA NPs in water) via i.p. injection once daily. During the experimental period, mouse weight and tumor volume were measured daily to determine IKE's antitumor effect and possible toxicity. Tumor growth was calculated as the fold change relative to original tumor volume on day 0 before the first dose (FIG. 4C). Administration of 40 mg/kg IKE, 23 mg/kg IKE, and 23 mg/kg IKE NPs caused a significant decrease in tumor growth starting from day 9 of treatment. The tumor growth inhibition effect was not significantly different between 23 mg/kg free IKE and 23 mg/kg IKE NPs; however, IKE NPs showed less toxicity, as evidenced by weight loss (FIG. 4D). Compared with saline vehicle, free IKE (5% DMSO in HBSS at pH 4)-treated mice started losing weight from day 9, which might be caused by the precipitation of IKE after administration into the peritoneum, an environment with pH ranges of 7.5-8.0, causing damage to abdominal organs, or possible toxicity of systemic system x_{c}⁻ inhibition, or off-target toxicity of IKE. However, IKE NP-treated mice had a similar weight compared with the saline vehicle and the NP vehicle groups; the lower toxicity of the IKE NP formulation might result from the NPs' capability to prevent the aggregation of hydrophobic drugs (Sun et al., 2014), or the NP EPR effect, which decreases the non-specific distribution and systemic toxicity associated with conventional hydrophobic drugs (Yue et al., 2013). By analyzing IKE tumor accumulation using liquid chromatography-MS, we found that IKE NPs at 23 mg/kg had slightly enhanced tumor accumulation compared with free IKE at 23 mg/kg and were comparable to the free IKE 40 mg/kg treatment (FIG. 10A). Overall, the PEG-PLGA NP formulation increased IKE's therapeutic window.

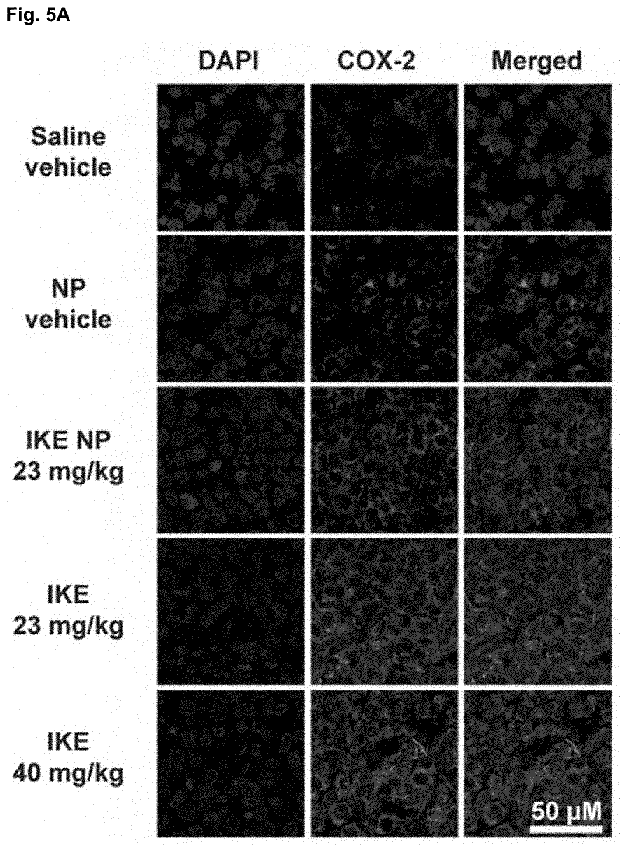

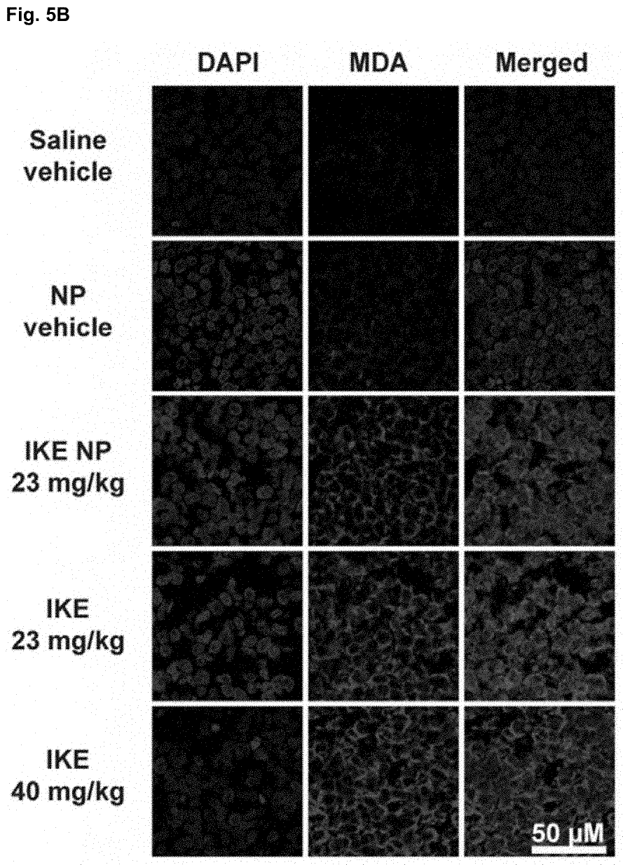

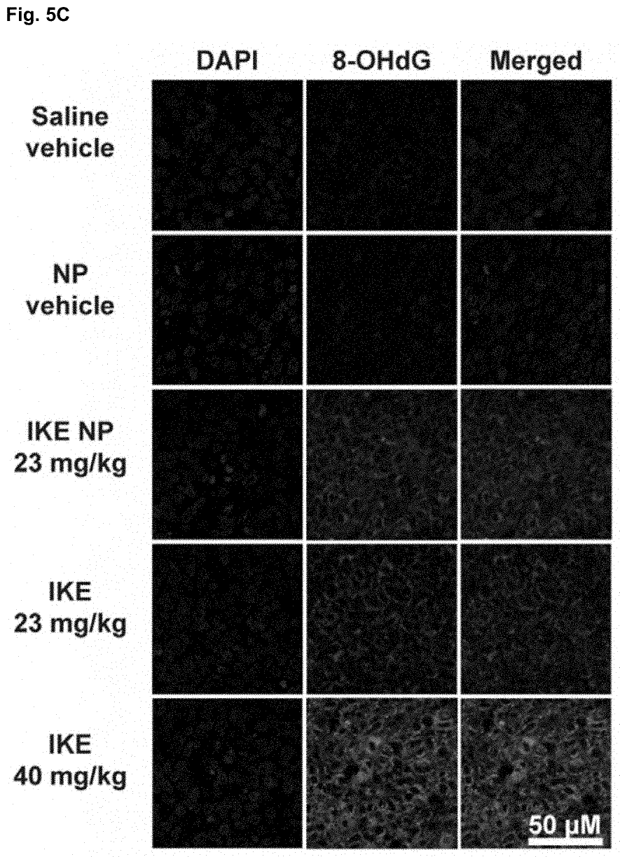

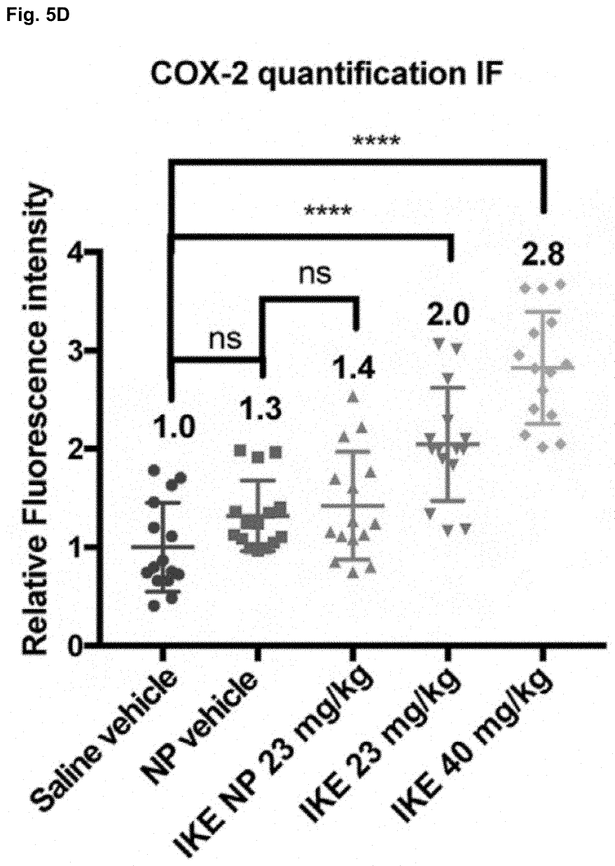

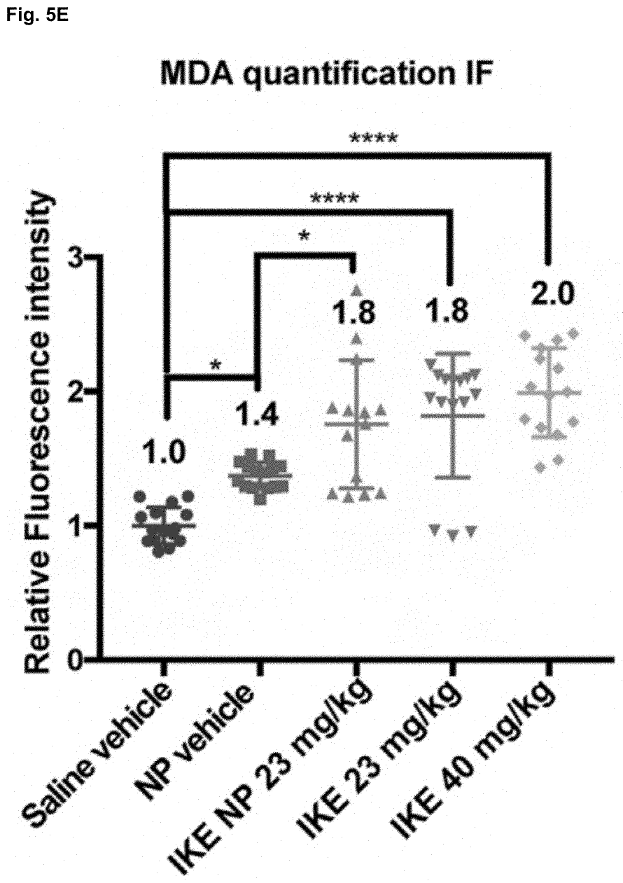

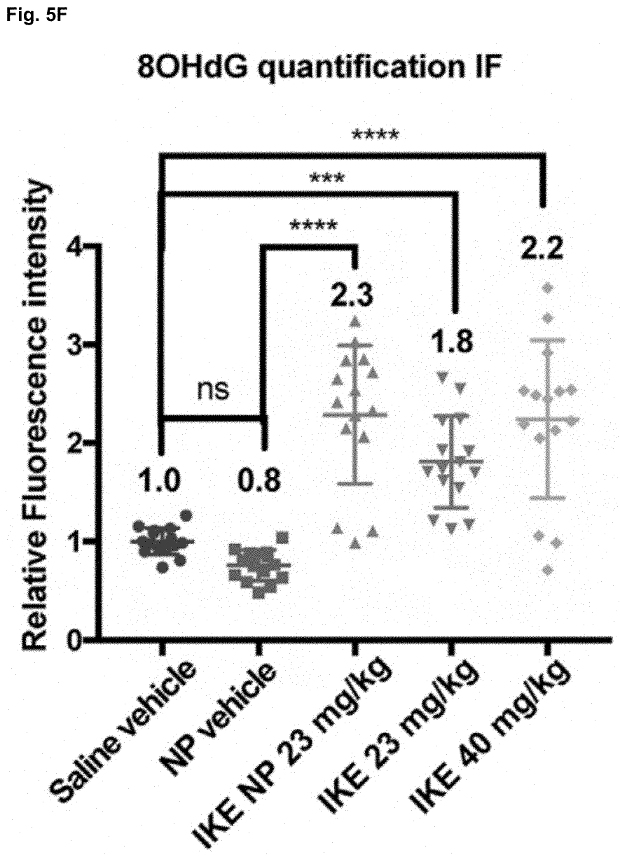

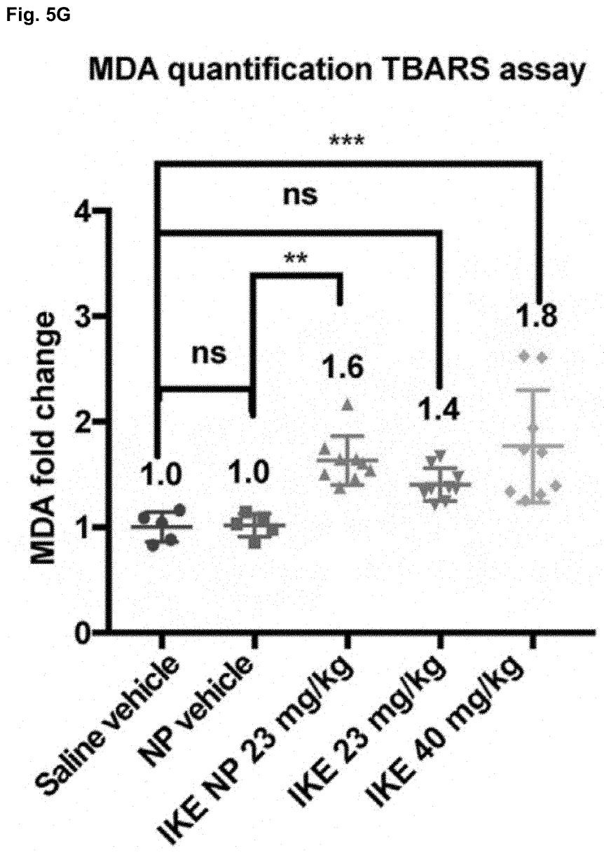

Given the fact that PTGS2 mRNA was upregulated upon IKE treatment in vivo (FIG. 3C), we hypothesized that after IKE treatment, there might be an increased level of the PTGS2 gene product (COX-2) in the tumor. Using immunofluorescence (FIG. 5A), we found that COX-2 protein expression was indeed increased in IKE-treated tumor tissue (FIG. 5D). There was no significant COX-2 protein expression increase in IKE NP-treated tumors, which might be due to the slower release of IKE from PEG-PLGA NPs (FIG. 9F), making the free IKE concentration lower than free IKE-treated tumors. This implies that COX-2 protein abundance is not a highly sensitive pharmacodynamic marker for low concentrations of IKE, but may more effectively report on exposure to high concentrations of IKE.

We further characterized lipid peroxidation in tumor tissues during this efficacy study. Immunofluorescence analysis of tumor tissue from the five groups showed significantly increased dihydropyridine-MDA-lysine adduct levels in IKE-treated and IKE PEG-PLGA NP-treated groups, compared with the saline vehicle and NP vehicle groups (FIGS. 5B and 5E). Compared with saline vehicle, the NP vehicle group had increased levels of dihydropyridine-MDA-lysine adducts, which might be due to low-level ferroptosis induction of ultrasmall (<10 nm in diameter) NPs in the sample, as observed in PEG-coated silica NPs previously (Kim et al., 2016). In addition, the thiobarbituric acid-reactive substances (TBARS) assay, which measures the fluorescence of MDA-TBA adducts formed, showed increased MDA levels in tumors in the free IKE and IKE PEG-PLGA NP groups compared with vehicle groups (FIG. 5G). The TBARS assay is not as sensitive as immunofluorescence for detecting small increases in MDA adducts, suggesting a possible explanation for our inability to detect this increase in the NP vehicle group. Immunofluorescence analysis of 8-OHdG showed increased oxidative DNA damage in IKE-treated and IKE PEG-PLGA NP-treated groups, confirming that IKE treatment induced oxidative damage (FIGS. 5C and 5F). In addition, there was no increase in caspase-3 activity in tumor tissues upon IKE treatment, suggesting that the inhibition of tumor growth was not due to apoptosis, as expected (FIG. 10B).

### Example 9

### Discussion

Recent discoveries have suggested that ferroptosis inducers can have antitumor efficacy (Yu et al., 2017; Gout et al., 2001; Liu et al., 2017) and may synergize with chemotherapy in some cell contexts (Sato et al., 2018; Chen et al., 2015; Yamaguchi et al., 2013; Yu et al., 2015). However, there is a lack of potent, selective, and metabolically stable tools to study ferroptosis in vivo. Study of the ferroptosis inducer IKE in an SUDHL6 xenograft model demonstrated that IKE and IKE NPs reduce tumor growth by inducing ferroptosis. The IKE PEG-PLGA NP formulation exhibited a larger therapeutic window compared with the free IKE formulation tested. Given the fact that we used a 5% DMSO in HBSS at pH 4 formulation and that IKE is less soluble at pH 7 versus pH 4, we suspect that the toxicity of high-dose free IKE in this study might stem from the precipitation of IKE after injection into the peritoneal cavity.

The induction of ferroptosis theoretically has the potential to shrink tumors. IKE's half-life (T_{1/2}) in plasma and T_{1/2} in tumor in SUDHL6 NCG xenograft mice through i.p. injection were 1.83 and 3.50 h, respectively. Compared with the pharmacokinetics of IKE, the once-per-day dosage frequency might be too low to see a tumor shrinking effect; in other words, we might need either a longer half-life compound or formulation or more frequent dosing to balance rapid tumor growth with induction of cell death, to cause tumor regression. The IKE NP formulation had increased accumulation in tumor tissue relative to plasma (FIG. 10A), but it might not be high enough to counter rapid tumor growth in vivo; delivery of a higher dose to the tumor might be needed. Based on the above considerations, combining IKE with other therapies or designing ferroptosis inducers with enhanced bioavailability and metabolic stability and higher tumor penetration and accumulation is worth investigating to further optimize the therapeutic impact of ferroptosis inducers in diverse cancer models.

Our untargeted lipidomic study identified lipid metabolism features during ferroptosis. First, we observed decreased PEs, PCs, and TAGs upon IKE treatment, possibly resulting from the cleavage of oxidized PUFA tails in PEs, PCs, and TAGs to prevent the oxidative damage to cells induced by IKE treatment. Co-treatment with β-ME, which allows transport of cysteine through systems A, ASC, and L, completely reversed the lipidomic changes caused by IKE treatment. Fer-1 partially prevented the phospholipid decreases in cells, but dramatically increased TAGs. The increased TAG pool might serve as a buffer against lipid peroxidation induced by IKE (Listenberger et al., 2003). However, the iron chelator DFO did not reverse IKE-induced phospholipid decreases or TAG decreases, indicating that Fenton chemistry mediated lipid peroxidation is not likely a driver of most lipidomic changes during IKE-induced ferroptosis. Second, both the de novo and the remodeling pathways of phospholipids and TAG biosynthesis (Shindou and Shimizu, 2009) were activated following IKE treatment. Finally, lipidomic changes following induction of ferroptosis in vivo were characterized here. The data from the tumor samples revealed that DAGs, MAGs, and phospholipids were all significantly increased upon IKE treatment, possibly resulting from the activation of the TAG hydrolysis enzyme, ATGL, and the response of tumor cells to oxidative stress.

In addition, pharmacodynamic markers developed in this study may be beneficial for future ferroptosis efficacy studies. First, the RT-qPCR experiments identified a time-dependent upregulation of numerous genes, including PTGS2, SLC7A11, and CHAC1, following IKE treatment in SUDHL6 cells and in xenograft tumor tissues derived from the same cell line. Second, immunofluorescence experiments showed increased levels of the PTGS2 gene product COX-2, 8-OHdG, and dihydropyridine-MDA-lysine adducts in IKE-treated and IKE PEG-PLGA NP-treated tumors compared with vehicle, with no increased caspase-3 cleavage, a biomarker of apoptosis, in IKE-treated and IKE PEG-PLGA NP-treated tumor tissues. These data collectively suggest that the antitumor effects of IKE result from system x_{c}⁻ inhibition-induced ferroptosis, but not apoptosis.

DLBCL cell lines have differential sensitivity to IKE-induced ferroptosis. The intrinsic factors determining DLBCL ferroptosis sensitivity remain to be explored to increase the understanding of ferroptosis and to enable selection of patients with sensitive tumors. The efficacy of IKE in additional animal cancer models would also be valuable to explore. Moreover, further studies on genes encoding lipid metabolism enzymes identified here would be useful to determine the essentiality of these genes for ferroptosis execution and the contribution of their expression to cell sensitivity to ferroptosis. In summary, we have developed a suitable small molecule and formulation for inhibiting system x_{c}⁻ and inducing ferroptosis in mouse tumor models and identified a series of pharmacodynamic markers of ferroptosis. These tools may be beneficial in determining whether there are specific cancer contexts in which ferroptosis induction would be therapeutically beneficial. The lipidomic study performed here may increase our understanding of the roles of lipids in ferroptotic cell death.

### DOCUMENTS CITED

1. Alizadeh, A. A.; Eisen, M. B.; Davis, R. E.; Ma, C.; Lossos, I. S.; Rosenwald, A.; Boldrick, J. C.; Sabet, H.; Tran, T.; Yu, X.; Powell, J. I.; Yang, L.; Marti, G. E.; Moore, T.; Hudson, J., Jr.; Lu, L.; Lewis, D. B.; Tibshirani, R.; Sherlock, G.; Chan, W. C.; Greiner, T. C.; Weisenburger, D. D.; Armitage, J. O.; Warnke, R.; Levy, R.; Wilson, W.; Grever, M. R.; Byrd, J. C.; Botstein, D.; Brown, P. O.; Staudt, L. M., Distinct types of diffuse large B-cell lymphoma identified by gene expression profiling. Nature 2000, 403 (6769), 503-11.
2. Blanco, E.; Shen, H.; Ferrari, M., Principles of nanoparticle design for overcoming biological barriers to drug delivery. Nature biotechnology 2015, 33 (9), 941.
3. Cajka, T.; Fiehn, O., Comprehensive analysis of lipids in biological systems by liquid chromatography-mass spectrometry. Trac-Trend Anal Chem 2014, 61, 192-206.
4. Chakraborti, S., Phospholipase A2 isoforms: a perspective. Cellular signalling 2003, 15 (7), 637-665.
5. Chapuy, B.; Stewart, C.; Dunford, A. J.; Kim, J.; Kamburov, A.; Redd, R. A.; Lawrence, M. S.; Roemer, M. G. M.; Li, A. J.; Ziepert, M.; Staiger, A. M.; Wala, J. A.; Ducar, M. D.; Leshchiner, I.; Rheinbay, E.; Taylor-Weiner, A.; Coughlin, C. A.; Hess, J. M.; Pedamallu, C. S.; Livitz, D.; Rosebrock, D.; Rosenberg, M.; Tracy, A. A.; Horn, H.; van Hummelen, P.; Feldman, A. L.; Link, B. K.; Novak, A. J.; Cerhan, J. R.; Habermann, T. M.; Siebert, R.; Rosenwald, A.; Thorner, A. R.; Meyerson, M. L.; Golub, T. R.; Beroukhim, R.; Wulf, G. G.; Ott, G.; Rodig, S. J.; Monti, S.; Neuberg, D. S.; Loeffler, M.; Pfreundschuh, M.; Trumper, L.; Getz, G.; Shipp, M. A., Molecular subtypes of diffuse large B cell lymphoma are associated with distinct pathogenic mechanisms and outcomes. Nat Med 2018, 24 (5), 679-690.
6. Chen, L.; Li, X.; Liu, L.; Yu, B.; Xue, Y.; Liu, Y., Erastin sensitizes glioblastoma cells to temozolomide by restraining xCT and cystathionine-γ-lyase function. Oncology reports 2015, 33 (3), 1465-1474.
7. Chong, J.; Soufan, O.; Li, C.; Caraus, I.; Li, S. Z.; Bourque, G.; Wishart, D. S.; Xia, J. G., MetaboAnalyst 4.0: towards more transparent and integrative metabolomics analysis. Nucleic Acids Res 2018, 46 (W1), W486-W494.
8. Dixon, S. J.; Lemberg, K. M.; Lamprecht, M. R.; Skouta, R.; Zaitsev, E. M.; Gleason, C. E.; Patel, D. N.; Bauer, A. J.; Cantley, A. M.; Yang, W. S., Ferroptosis: an iron-dependent form of nonapoptotic cell death. Cell 2012, 149 (5), 1060-1072.
9. Dixon, S. J.; Patel, D. N.; Welsch, M.; Skouta, R.; Lee, E. D.; Hayano, M.; Thomas, A. G.; Gleason, C. E.; Tatonetti, N. P.; Slusher, B. S., Pharmacological inhibition of cysteine-glutamate exchange induces endoplasmic reticulum stress and ferroptosis. Elife 2014, 3.
10. Doll, S., Proneth, B., Tyurina, Y. Y., Panzilius, E., Kobayashi, S., Ingold, I., Irmler, M., Beckers, J., Aichler, M., Walch, A., et al. (2017). ACSL4 dictates ferroptosis sensitivity by shaping cellular lipid composition. Nat. Chem. Biol. 13, 91-98.
11. Galluzzi, L.; Vitale, I.; Aaronson, S. A.; Abrams, J. M.; Adam, D.; Agostinis, P.; Alnemri, E. S.; Altucci, L.; Amelio, I.; Andrews, D. W.; Annicchiarico-Petruzzelli, M.; Antonov, A. V.; Arama, E.; Baehrecke, E. H.; Barlev, N. A.; Bazan, N. G.; Bernassola, F.; Bertrand, M. J. M.; Bianchi, K.; Blagosklonny, M. V.; Blomgren, K.; Borner, C.; Boya, P.; Brenner, C.; Campanella, M.; Candi, E.; Carmona-Gutierrez, D.; Cecconi, F.; Chan, F. K.; Chandel, N. S.; Cheng, E. H.; Chipuk, J. E.; Cidlowski, J. A.; Ciechanover, A.; Cohen, G. M.; Conrad, M.; Cubillos-Ruiz, J. R.; Czabotar, P. E.; D'Angiolella, V.; Dawson, T. M.; Dawson, V. L.; De Laurenzi, V.; De Maria, R.; Debatin, K. M.; DeBerardinis, R. J.; Deshmukh, M.; Di Daniele, N.; Di Virgilio, F.; Dixit, V. M.; Dixon, S. J.; Duckett, C. S.; Dynlacht, B. D.; EI-Deiry, W. S.; Elrod, J. W.; Fimia, G. M.; Fulda, S.; Garcia-Saez, A. J.; Garg, A. D.; Garrido, C.; Gavathiotis, E.; Golstein, P.; Gottlieb, E.; Green, D. R.; Greene, L. A.; Gronemeyer, H.; Gross, A.; Hajnoczky, G.; Hardwick, J. M.; Harris, I. S.; Hengartner, M. O.; Hetz, C.; Ichijo, H.; Jaattela, M.; Joseph, B.; Jost, P. J.; Juin, P. P.; Kaiser, W. J.; Karin, M.; Kaufmann, T.; Kepp, O.; Kimchi, A.; Kitsis, R. N.; Klionsky, D. J.; Knight, R. A.; Kumar, S.; Lee, S. W.; Lemasters, J. J.; Levine, B.; Linkermann, A.; Lipton, S. A.; Lockshin, R. A.; Lopez-Otin, C.; Lowe, S. W.; Luedde, T.; Lugli, E.; MacFarlane, M.; Madeo, F.; Malewicz, M.; Malorni, W.; Manic, G.; Marine, J. C.; Martin, S. J.; Martinou, J. C.; Medema, J. P.; Mehlen, P.; Meier, P.; Melino, S.; Miao, E. A.; Molkentin, J. D.; Moll, U. M.; Munoz-Pinedo, C.; Nagata, S.; Nunez, G.; Oberst, A.; Oren, M.; Overholtzer, M.; Pagano, M.; Panaretakis, T.; Pasparakis, M.; Penninger, J. M.; Pereira, D. M.; Pervaiz, S.; Peter, M. E.; Piacentini, M.; Pinton, P.; Prehn, J. H. M.; Puthalakath, H.; Rabinovich, G. A.; Rehm, M.; Rizzuto, R.; Rodrigues, C. M. P.; Rubinsztein, D. C.; Rudel, T.; Ryan, K. M.; Sayan, E.; Scorrano, L.; Shao, F.; Shi, Y.; Silke, J.; Simon, H. U.; Sistigu, A.; Stockwell, B. R.; Strasser, A.; Szabadkai, G.; Tait, S. W. G.; Tang, D.; Tavernarakis, N.; Thorburn, A.; Tsujimoto, Y.; Turk, B.; Vanden Berghe, T.; Vandenabeele, P.; Vander Heiden, M. G.; Villunger, A.; Virgin, H. W.; Vousden, K. H.; Vucic, D.; Wagner, E. F.; Walczak, H.; Wallach, D.; Wang, Y.; Wells, J. A.; Wood, W.; Yuan, J.; Zakeri, Z.; Zhivotovsky, B.; Zitvogel, L.; Melino, G.; Kroemer, G., Molecular mechanisms of cell death: recommendations of the Nomenclature Committee on Cell Death 2018. Cell Death Differ 2018, 25 (3), 486-541.
12. Gaschler, M. M., and Stockwell, B. R. (2017). Lipid peroxidation in cell death. Biochem. Biophys. Res. Commun. 482, 419-425.
13. Gdowski, A.; Johnson, K.; Shah, S.; Gryczynski, I.; Vishwanatha, J.; Ranjan, A., Optimization and scale up of microfluidic nanolipomer production method for preclinical and potential clinical trials. J Nanobiotechnol 2018, 16.
14. Goracci, L.; Tortorella, S.; Tiberi, P.; Pellegrino, R. M.; Di Veroli, A.; Valeri, A.; Cruciani, G., Lipostar, a comprehensive platform-neutral cheminformatics tool for lipidomics. Analytical chemistry 2017, 89 (11), 6257-6264.
15. Gout, P.; Buckley, A.; Simms, C.; Bruchovsky, N., Sulfasalazine, a potent suppressor of lymphoma growth by inhibition of the x c− cysteine transporter: a new action for an old drug. Leukemia 2001, 15 (10), 1633.
16. Guan, J., Lo, M., Dockery, P., Mahon, S., Karp, C. M., Buckley, A. R., Lam, S., Gout, P. W., and Wang, Y.-Z. (2009). The x c cysteine/glutamate antiporter as a potential therapeutic target for small-cell lung cancer: use of sulfasalazine. Cancer Chemother. Pharmacol. 64, 463.
17. Hyvarinen, S.; Uchida, K.; Varjosalo, M.; Jokela, R.; Jokiranta, T. S., Recognition of Malondialdehyde-modified Proteins by the C Terminus of Complement Factor H Is Mediated via the Polyanion Binding Site and Impaired by Mutations Found in Atypical Hemolytic Uremic Syndrome. Journal of Biological Chemistry 2014, 289 (7), 4295-4306.
18. Kagan, V. E., Mao, G., Qu, F., Angeli, J. P. F., Doll, S., St Croix, C., Dar, H. H., Liu, B., Tyurin, V. A., and Ritov, V. B. (2017). Oxidized arachidonic and adrenic PEs navigate cells to ferroptosis. Nat. Chem. Biol. 13, 81.
19. Kim, S. E.; Zhang, L.; Ma, K.; Riegman, M.; Chen, F.; Ingold, I.; Conrad, M.; Turker, M. Z.; Gao, M.; Jiang, X.; Monette, S.; Pauliah, M.; Gonen, M.; Zanzonico, P.; Quinn, T.; Wiesner, U.; Bradbury, M. S.; Overholtzer, M., Ultrasmall nanoparticles induce ferroptosis in nutrient-deprived cancer cells and suppress tumour growth. Nat Nanotechnol 2016, 11 (11), 977-985.
20. Larraufie, M.-H.; Yang, W. S.; Jiang, E.; Thomas, A. G.; Slusher, B. S.; Stockwell, B. R., Incorporation of metabolically stable ketones into a small molecule probe to increase potency and water solubility. Bioorganic & medicinal chemistry letters 2015, 25 (21), 4787-4792.
21. Listenberger, L. L., Han, X. L., Lewis, S. E., Cases, S., Farese, R. V., Ory, D. S., and Schaffer, J. E. (2003). Triglyceride accumulation protects against fatty acid-induced lipotoxicity. Proc. Natl. Acad. Sci. USA 100, 3077-3082.
22. Liu, D. S.; Duong, C. P.; Haupt, S.; Montgomery, K. G.; House, C. M.; Azar, W. J.; Pearson, H. B.; Fisher, O. M.; Read, M.; Guerra, G. R.; Haupt, Y.; Cullinane, C.; Wiman, K. G.; Abrahmsen, L.; Phillips, W. A.; Clemons, N. J., Inhibiting the system xC(−)/glutathione axis selectively targets cancers with mutant-p53 accumulation. Nat Commun 2017, 8, 14844.
23. Lo, M., Ling, V., Low, C., Wang, Y., and Gout, P. (2010). Potential use of the anti-inflammatory drug, sulfasalazine, for targeted therapy of pancreatic cancer. Curr. Oncol. 17, 9.
24. Lu, B., Chen, X. B., Ying, M. D., He, Q. J., Cao, J., and Yang, B. (2018). The role of ferroptosis in cancer development and treatment response. Front. Pharmacol. 8, 992.
25. Magtanong, L., Ko, P. J., and Dixon, S. J. (2016). Emerging roles for lipids in nonapoptotic cell death. Cell Death Differ. 23, 1099-1109.
26. Matyash, V.; Liebisch, G.; Kurzchalia, T. V.; Shevchenko, A.; Schwudke, D., Lipid extraction by methyl-tert-butyl ether for high-throughput lipidomics. J Lipid Res 2008, 49 (5), 1137-46.
27. McCullagh, E. A., and Featherstone, D. E. (2014). Behavioral characterization of system xc− mutant mice. Behav. Brain Res. 265, 1-11.
28. Narang, V. S.; Pauletti, G. M.; Gout, P. W.; Buckley, D. J.; Buckley, A. R., Sulfasalazine-induced reduction of glutathione levels in breast cancer cells: enhancement of growth-inhibitory activity of Doxorubicin. Chemotherapy 2007, 53 (3), 210-7.
29. Peer, D.; Karp, J. M.; Hong, S.; Farokhzad, O. C.; Margalit, R.; Langer, R., Nanocarriers as an emerging platform for cancer therapy. Nature nanotechnology 2007, 2 (12), 751.
30. Sato, M.; Kusumi, R.; Hamashima, S.; Kobayashi, S.; Sasaki, S.; Komiyama, Y.; Izumikawa, T.; Conrad, M.; Bannai, S.; Sato, H., The ferroptosis inducer erastin irreversibly inhibits system xc− and synergizes with cisplatin to increase cisplatin's cytotoxicity in cancer cells. Scientific reports 2018, 8 (1), 968.
31. Sengupta, A.; Lichti, U. F.; Carlson, B. A.; Cataisson, C.; Ryscavage, A. O.; Mikulec, C.; Conrad, M.; Fischer, S. M.; Hatfield, D. L.; Yuspa, S. H., Targeted disruption of glutathione peroxidase 4 in mouse skin epithelial cells impairs postnatal hair follicle morphogenesis that is partially rescued through inhibition of COX-2. Journal of Investigative Dermatology 2013, 133 (7), 1731-1741.
32. Shindou, H., and Shimizu, T. (2009). Acyl-CoA: lysophospholipid acyltransferases. J. Biol. Chem. 284, 1-5.
33. Skouta, R.; Dixon, S. J.; Wang, J.; Dunn, D. E.; Orman, M.; Shimada, K.; Rosenberg, P. A.; Lo, D. C.; Weinberg, J. M.; Linkermann, A., Ferrostatins inhibit oxidative lipid damage and cell death in diverse disease models. Journal of the American Chemical Society 2014, 136 (12), 4551-4556.
34. Smith, C. A.; Want, E. J.; O'Maille, G.; Abagyan, R.; Siuzdak, G., XCMS: Processing mass spectrometry data for metabolite profiling using Nonlinear peak alignment, matching, and identification. Anal Chem 2006, 78 (3), 779-787.
35. Stockwell, B. R.; Angeli, J. P. F.; Bayir, H.; Bush, A. I.; Conrad, M.; Dixon, S. J.; Fulda, S.; Gascón, S.; Hatzios, S. K.; Kagan, V. E., Ferroptosis: a regulated cell death nexus linking metabolism, redox biology, and disease. Cell 2017, 171 (2), 273-285.
36. Sun, T.; Zhang, Y. S.; Pang, B.; Hyun, D. C.; Yang, M.; Xia, Y., Engineered nanoparticles for drug delivery in cancer therapy. Angewandte Chemie International Edition 2014, 53 (46), 12320-12364.
37. Tautenhahn, R.; Bottcher, C.; Neumann, S., Highly sensitive feature detection for high resolution LC/MS. Bmc Bioinformatics 2008, 9.
38. Uchida, K., HNE as an inducer of COX-2. Free Radical Biology and Medicine 2017, 111, 169-172.
39. Valencia, P. M.; Pridgen, E. M.; Rhee, M.; Langer, R.; Farokhzad, O. C.; Karnik, R., Microfluidic platform for combinatorial synthesis and optimization of targeted nanoparticles for cancer therapy. ACS Nano 2013, 7 (12), 10671-80.
40. Yamada, S.; Kumazawa, S.; Ishii, T.; Nakayama, T.; Itakura, K.; Shibata, N.; Kobayashi, M.; Sakai, K.; Osawa, T.; Uchida, K., Immunochemical detection of a lipofuscin-like fluorophore derived from malondialdehyde and lysine. J Lipid Res 2001, 42 (8), 1187-1196.
41. Yamaguchi, H.; Hsu, J. L.; Chen, C.-T.; Wang, Y.-N.; Hsu, M.-C.; Chang, S.-S.; Du, Y.; Ko, H.-W.; Herbst, R.; Hung, M.-C., Caspase-Independent Cell Death Is Involved in the Negative Effect of EGF Receptor Inhibitors on Cisplatin in Non-Small Cell Lung Cancer Cells. Clinical cancer research 2013.
42. Yang, W. S.; Kim, K. J.; Gaschler, M. M.; Patel, M.; Shchepinov, M. S.; Stockwell, B. R., Peroxidation of polyunsaturated fatty acids by lipoxygenases drives ferroptosis. Proceedings of the National Academy of Sciences 2016, 113 (34), E4966-E4975.
43. Yang, W. S.; SriRamaratnam, R.; Welsch, M. E.; Shimada, K.; Skouta, R.; Viswanathan, V. S.; Cheah, J. H.; Clemons, P. A.; Shamji, A. F.; Clish, C. B., Regulation of ferroptotic cancer cell death by GPX4. Cell 2014, 156 (1), 317-331.
44. Yang, W. S.; Stockwell, B. R., Ferroptosis: death by lipid peroxidation. Trends in cell biology 2016, 26 (3), 165-176.
45. Yu, H. T.; Guo, P. Y.; Xie, X. Z.; Wang, Y.; Chen, G., Ferroptosis, a new form of cell death, and its relationships with tumourous diseases. Journal of Cellular and Molecular Medicine 2017, 21 (4), 648-657.
46. Yu, Y.; Xie, Y.; Cao, L.; Yang, L.; Yang, M.; Lotze, M. T.; Zeh, H. J.; Kang, R.; Tang, D., The ferroptosis inducer erastin enhances sensitivity of acute myeloid leukemia cells to chemotherapeutic agents. Molecular & cellular oncology 2015, 2 (4), e1054549.
47. Yue, Z., You, Z., Yang, Q., Lv, P., Yue, H., Wang, B., Ni, D., Su, Z., Wei, W., and Ma, G. (2013). Molecular structure matters: PEG-b-PLA nanoparticles with hydrophilicity and deformability demonstrate their advantages for high-performance delivery of anti-cancer drugs. J. Mater. Chem. B 1, 3239-3247.
48. Zilka, O.; Shah, R.; Li, B.; Friedmann Angeli, J. P.; Griesser, M.; Conrad, M.; Pratt, D. A., On the Mechanism of Cytoprotection by Ferrostatin-1 and Liproxstatin-1 and the Role of Lipid Peroxidation in Ferroptotic Cell Death. ACS Cent Sci 2017, 3 (3), 232-243.

All documents cited in this application are hereby incorporated by reference as if recited in full herein.

Although illustrative embodiments of the present disclosure have been described herein, it should be understood that the disclosure is not limited to those described, and that various other changes or modifications may be made by one skilled in the art without departing from the scope or spirit of the disclosure.

What is claimed is:

1. A nanoparticle formulation comprising nanoparticles of a polymer loaded with a system x_{c}⁻ inhibitor.

2. The nanoparticle formulation of claim 1, wherein the polymer is biodegradable.

3. The nanoparticle formulation of claim 1, wherein the polymer is selected from poly(lactic acid) (PLA), poly(lactide-co-glycolide) (PLGA), and poly(ethylene glycol)-poly(lactic-co-glycolic acid) (PEG-PLGA).

4. The nanoparticle formulation of claim 1, wherein the polymer is poly(ethylene glycol)-poly(lactic-co-glycolic acid) (PEG-PLGA).

5. The nanoparticle formulation of claim 1, wherein the system x_{c}⁻ inhibitor is a small molecule.

6. The nanoparticle formulation of claim 1, wherein the system x_{c}⁻ inhibitor is an erastin analog.

7. The nanoparticle formulation of claim 1, wherein the system x_{c}⁻ inhibitor is selected from 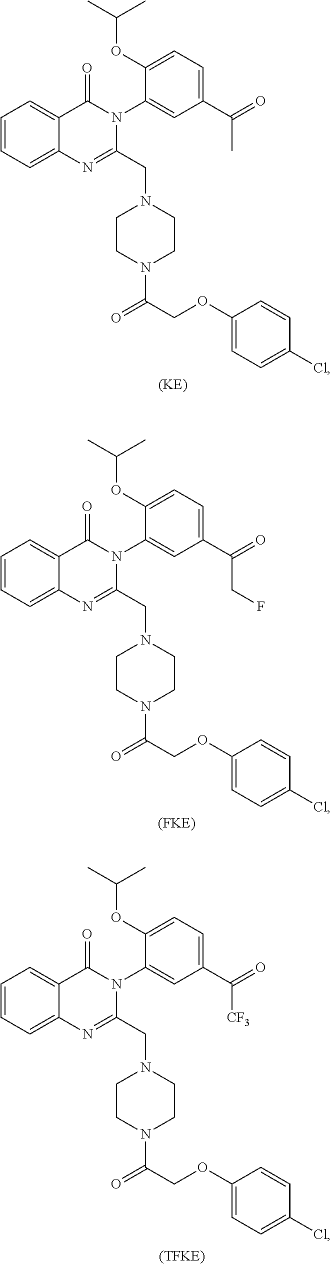 [SMILES: CC(=O)c1ccc(OC(C)C)c(-n2c(CN3CCN(C(=O)COc4ccc(Cl)cc4)CC3)nc3ccccc3c2=O)c1 ; CC(C)Oc1ccc(C(=O)CF)cc1-n1c(CN2CCN(C(=O)COc3ccc(Cl)cc3)CC2)nc2ccccc2c1=O ; CC(C)Oc1ccc(C(=O)C(F)(F)F)cc1-n1c(CN2CCN(C(=O)COc3ccc(Cl)cc3)CC2)nc2ccccc2c1=O] 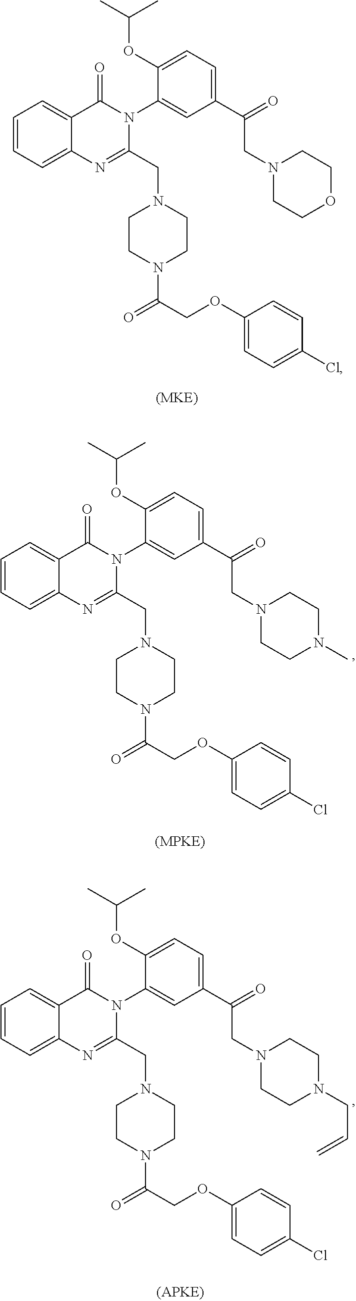 [SMILES: CC(C)Oc1ccc(C(=O)CN2CCOCC2)cc1-n1c(CN2CCN(C(=O)COc3ccc(Cl)cc3)CC2)nc2ccccc2c1=O ; CC(C)Oc1ccc(C(=O)CN2CCN(C)CC2)cc1-n1c(CN2CCN(C(=O)COc3ccc(Cl)cc3)CC2)nc2ccccc2c1=O ; C=CCN1CCN(CC(=O)c2ccc(OC(C)C)c(-n3c(CN4CCN(C(=O)COc5ccc(Cl)cc5)CC4)nc4ccccc4c3=O)c2)CC1] 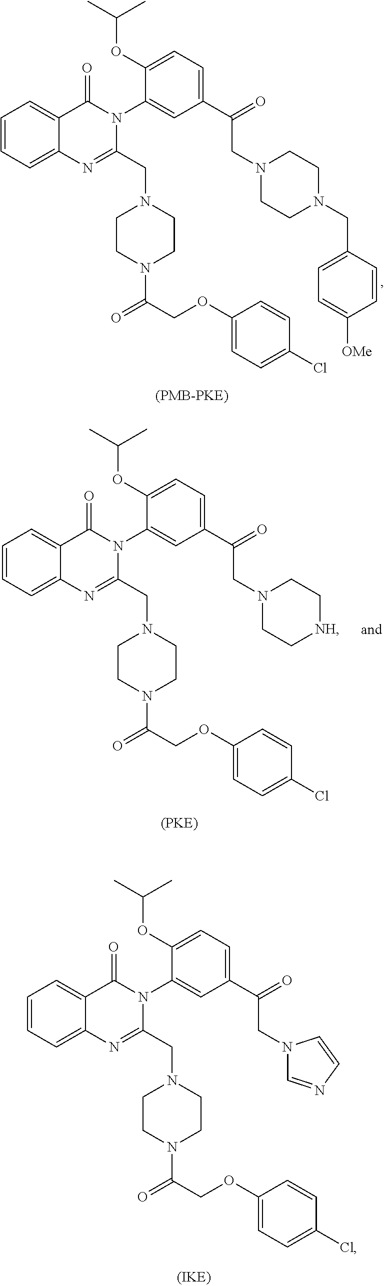 [SMILES: COc1ccc(CN2CCN(CC(=O)c3ccc(OC(C)C)c(-n4c(CN5CCN(C(=O)COc6ccc(Cl)cc6)CC5)nc5ccccc5c4=O)c3)CC2)cc1 ; CC(C)Oc1ccc(C(=O)CN2CCNCC2)cc1-n1c(CN2CCN(C(=O)COc3ccc(Cl)cc3)CC2)nc2ccccc2c1=O ; "and" ; CC(C)Oc1ccc(C(=O)Cn2ccnc2)cc1-n1c(CN2CCN(C(=O)COc3ccc(Cl)cc3)CC2)nc2ccccc2c1=O]
or pharmaceutically acceptable salts thereof.

8. The nanoparticle formulation of claim 1, wherein the system x_{c}⁻ inhibitor is IKE or pharmaceutically acceptable salts thereof.

9. The nanoparticle formulation of claim 1, wherein the loaded nanoparticle has a size between 20 nm and 200 nm.

10. The nanoparticle formulation of claim 1, wherein the loaded nanoparticle has a size of about 80 nm.

11. The nanoparticle formulation of claim 1, wherein the loaded nanoparticle has a surface potential of about −17 mV.

12. The nanoparticle formulation of claim 1, having a polydispersity index of about 0.2.

13. The nanoparticle formulation of claim 1, having an encapsulation efficiency of about 24%.

14. A nanoparticle formulation comprising nanoparticles of PEG-PLGA loaded with IKE or a pharmaceutically acceptable salt thereof.

15. A method of preparing the nanoparticle formulation according to claim 14, comprising the steps of:
(a) assembling the nanoparticles by employing a NanoAssemblr platform equipped with a high flow microfluidic chip, using the following settings:
i) 1:1 ratio of organic to aqueous phases,
ii) 25% acetone/75% dimethyl sulfoxide (DMSO) as the organic phase, 10 mg/mL poly(ethylene glycol)-poly(lactic-co-glycolic acid) (PEG-PLGA) in organic phase, and 15% (by weight) IKE to PEG-PLGA polymer in the organic phase;
iii) pure water as the aqueous phase;
iv) total flow rate of 8 mL/min; and
(b) concentrating the assembled nanoparticles by using filter units with concentration factors up to 20.

16. A method for treating or ameliorating the effects of a cancer in a subject, comprising administering to the subject a therapeutically effective amount of a nanoparticle formulation according to any one of claims 1 to 14.

17. The method of claim 16, wherein the cancer is diffuse large B cell lymphoma (DLBCL).

18. The method of claim 16, wherein the nanoparticle formulation is administered at up to 750 mg/kg per day.

19. The method of claim 16, further comprising co-administering to the subject a chemotherapy drug selected from the group consisting of cisplatin, temozolomide, doxorubicin, cyclophosphamide, methotrexate, 5-fluorouracil, vinorelbine, docetaxel, bleomycin, vinblastine, dacarbazine, mustine, vincristine, procarbazine, prednisolone, etoposide, epirubicin, capecitabine, methotrexate, folinic acid, oxaliplatin, and combinations thereof.

20. A method for selectively killing a cancer cell, comprising contacting the cancer cell with an effective amount of a nanoparticle formulation according to any one of claims 1 to 14.

21. A kit comprising a nanoparticle formulation according to any one of claims 1 to 14 together with instructions for the use of the nanoparticle formulation.


SEQUENCE LISTING (36 sequences)

SEQ ID NO: 1 - DNA, 21 bp, Artificial Sequence
  forward primer
gccctggcgtcgtgattagtg

SEQ ID NO: 2 - DNA, 21 bp, Artificial Sequence
  reverse primer
gcctcccatctccttcatcac

SEQ ID NO: 3 - DNA, 19 bp, Artificial Sequence
  forward primer
gaaccctggttacctgggc

SEQ ID NO: 4 - DNA, 22 bp, Artificial Sequence
  reverse primer
cgcagcaagtattcaaggttgt

SEQ ID NO: 5 - DNA, 18 bp, Artificial Sequence
  forward primer
ggtggtgtgtttgctgtc

SEQ ID NO: 6 - DNA, 18 bp, Artificial Sequence
  reverse primer
gctggtagaggagtgtgc

SEQ ID NO: 7 - DNA, 22 bp, Artificial Sequence
  forward primer
tatgttctcctgcctactggaa

SEQ ID NO: 8 - DNA, 22 bp, Artificial Sequence
  reverse primer
gcccttcacgttattgcagatg

SEQ ID NO: 9 - DNA, 20 bp, Artificial Sequence
  forward primer
ccccgctatctctcagacac

SEQ ID NO: 10 - DNA, 19 bp, Artificial Sequence
  reverse primer
ggtgctccaactctgccag

SEQ ID NO: 11 - DNA, 22 bp, Artificial Sequence
  forward primer
cttccgagattccatcctacgc

SEQ ID NO: 12 - DNA, 21 bp, Artificial Sequence
  reverse primer
tggcagtcaggctcacaaacg

SEQ ID NO: 13 - DNA, 25 bp, Artificial Sequence
  forward primer
tccctctacattgtctatgagttcc

SEQ ID NO: 14 - DNA, 28 bp, Artificial Sequence
  reverse primer
ttcagttggccttgaccttggcaatacc

SEQ ID NO: 15 - DNA, 34 bp, Artificial Sequence
  forward primer
aggatccatggccttcagtgatcttacatcgagg

SEQ ID NO: 16 - DNA, 25 bp, Artificial Sequence
  reverse primer
aaccacctgcagcaaatttgactcc

SEQ ID NO: 17 - DNA, 29 bp, Artificial Sequence
  forward primer
agaaaagttgactagtccagtgtggtgaa

SEQ ID NO: 18 - DNA, 36 bp, Artificial Sequence
  reverse primer
aaaagctgtgctaaaccaattccgaacagattctca

SEQ ID NO: 19 - DNA, 21 bp, Artificial Sequence
  forward primer
ggagccttcctaacctacagc

SEQ ID NO: 20 - DNA, 22 bp, Artificial Sequence
  reverse primer
ctcacgattccttccacatacc

SEQ ID NO: 21 - DNA, 21 bp, Artificial Sequence
  forward primer
cacaaccaagtggaaatcgag

SEQ ID NO: 22 - DNA, 18 bp, Artificial Sequence
  reverse primer
gcacgttgctggcataca

SEQ ID NO: 23 - DNA, 20 bp, Artificial Sequence
  forward primer
atcactgccgtcctcactac

SEQ ID NO: 24 - DNA, 20 bp, Artificial Sequence
  reverse primer
agtcaacagccaaaccaatc

SEQ ID NO: 25 - DNA, 20 bp, Artificial Sequence
  forward primer
ggtgggagagaatgccactt

SEQ ID NO: 26 - DNA, 20 bp, Artificial Sequence
  reverse primer
atgcaagggatgatggctgt

SEQ ID NO: 27 - DNA, 23 bp, Artificial Sequence
  forward primer
ctgaaatgtgtgtgctatgagcg

SEQ ID NO: 28 - DNA, 23 bp, Artificial Sequence
  reverse primer
tggaagagaggaagtggtgtctg

SEQ ID NO: 29 - DNA, 22 bp, Artificial Sequence
  forward primer
gagggctaggtctttctggaag

SEQ ID NO: 30 - DNA, 22 bp, Artificial Sequence
  reverse primer
ccacagtgaaatctcgttgaga

SEQ ID NO: 31 - DNA, 24 bp, Artificial Sequence
  forward primer
tgacgacaggaaaggaagccgcac

SEQ ID NO: 32 - DNA, 25 bp, Artificial Sequence
  reverse primer
agggaagaggggactcagcaacgag

SEQ ID NO: 33 - DNA, 20 bp, Artificial Sequence
  forward primer
ctggaacctgtgttggacct

SEQ ID NO: 34 - DNA, 20 bp, Artificial Sequence
  reverse primer
cggtgatatctgtggtcacg

SEQ ID NO: 35 - DNA, 21 bp, Artificial Sequence
  forward primer
caacgccactcacatctacgg

SEQ ID NO: 36 - DNA, 23 bp, Artificial Sequence
  reverse primer
ggacacctcaataatgttggcac